(19) United States
(12) Patent Application Publication
(10) Pub. No.: US 2026/0285906 A1
(43) Pub. Date: Sep. 24, 2026

(54) MASKING POLYPEPTIDES, ACTIVATABLE CYTOKINE CONSTRUCTS, AND RELATED COMPOSITIONS AND METHODS

(71) Applicant: CytomX Therapeutics, Inc., South San Francisco, CA (US)

(72) Inventors: Pankaj KUMAR, San Bruno, CA (US); Na CAI, San Mateo, CA (US); Erwan LE SCOLAN, San Francisco, CA (US); Madan M. PAIDHUNGAT, San Francisco, CA (US); Dylan L. DANIEL, San Francisco, CA (US)

(21) Appl. No.: 19/474,388

(22) PCT Filed: Apr. 12, 2024
(86) PCT No.: PCT/US2024/024476

§ 371 Date: Oct. 10, 2025

Related U.S. Application Data
(60) Provisional application No. 63/495,683, filed on Apr. 12, 2023.

(51) Int. Cl. C07K 7/08; A61K 38/00; C07K 14/54; C12N 15/85

(52) U.S. Cl. CPC C07K 7/08; C07K 14/5443; C12N 15/85; A61K 38/00; C07K 2319/21; C07K 2319/30

(57) ABSTRACT

Provided herein are activatable cytokine constructs that include a novel masking moiety, a cytokine polypeptide, and a cleavable moiety between the masking moiety and the cytokine polypeptide. In some embodiments, the ACC is a monomer. In some embodiments, the ACC is a complex of two, three, four, or more constructs. In some embodiments, the ACC is a dimer complex comprising a first monomer construct comprising a first cytokine polypeptide, a first masking moiety, and a first dimerization domain, and a second monomer construct comprising a second cytokine polypeptide and/or an agonist of the first cytokine polypeptide and a second dimerization domain, and optionally a second masking moiety, wherein the first and/or second masking moiety is the novel masking moiety.

### CROSS-REFERENCE TO RELATED APPLICATION

This application claims the priority benefit of U.S. provisional application No. 63/495,683 filed Apr. 12, 2023, the contents of which are incorporated herein in their entireties by reference thereto.

### SEQUENCE LISTING

The instant application contains a Sequence Listing which has been submitted electronically in XML format and is hereby incorporated by reference in its entirety. Said XML copy, created on Apr. 12, 2024 is named “4862-145.xml” and is 659,456 bytes in size.

### TECHNICAL FIELD

The present disclosure relates to the field of biotechnology, and more specifically, to isolated polypeptides and activatable cytokine constructs, including activatable interleukin constructs.

### BACKGROUND

Cytokines are a family of naturally-occurring small proteins and glycoproteins produced and secreted by most nucleated cells in response to viral infection and/or other antigenic stimuli. Interleukins are a subclass of cytokines. Interleukins regulate cell growth, differentiation, and motility. They are particularly important in stimulating immune responses, such as inflammation. Interleukins have been used for treatment of cancer, autoimmune disorders, and other disorders. For example, interleukin-2 (IL2) is indicated for treatment of melamona, graft-versus-host disease (GVHD), neuroblastoma, renal cell cancer (RCC), and is also considered useful for conditions including acute coronary syndrome, acute myeloid syndrome, atopic dermatitis, autoimmune liver diseases, basal cell carcinoma, bladder cancer, breast cancer, candidiasis, colorectal cancer, cutaneous T-cell lymphoma, endometriomas, HIV infection, ischemic heart disease, rheumatoid arthritis, nasopharyngeal adenocarcimoa, non-small cell lung cancer (NSCLC), ovarian cancer, pancreatic cancer, systemic lupus erythematosus, tuberculosis, and other disorders. Interleukin-15 (IL-15) is known to promote the differentiation and expansion of T cells, B cells and natural killer (NK) cells, leading to enhanced antitumor responses. IL-15 has been identified as a promising candidate for anticancer therapy, and it has been tested in numerous clinical trials. Despite this promise, IL-15 is known to exhibit unwanted pro-inflammatory effects and has been associated with the pathogenesis of several autoimmune diseases. Recombinant IL-15 has been reported as having a maximum tolerated dose of 2 micrograms/kg. Conlon K C, et al. “IL15 by Continuous Intravenous Infusion to Adult Patients with Solid Tumors in a Phase I Trial Induced Dramatic NK-Cell Subset Expansion.” Clin Cancer Res. 2019 Aug. 15; 25(16):4945-4954. Recombinant soluble IL-15 also has been reported as having a short half-life in vivo, which has hampered its use as a therapeutic. Berraondo, P., et al. “Cytokines in clinical cancer immunotherapy.” Br J Cancer 120, 6-15 (2019). Other interleukins, such as IL-4, IL-6, IL-7, IL-9, IL-12, and IL-21, among others, are also potential treatments for cancers and other disorders. Interleukin therapy, however, is often accompanied by undesired side effects, including flu-like symptoms, nausea, vomiting, diarrhea, low blood pressure, and arrhythmia, among others.

Accordingly, there is a continuing need for cytokine therapies that have fewer of the undesired side effects of existing cytokine therapeutics.

### SUMMARY

The present disclosure provides isolated polypeptide and activatable cytokine constructs (ACC) that include one or more novel masking moieties.

In one aspect, the present disclosure includes an isolated polypeptide comprising amino acid sequence X₁LTTVX₂-linker-ASHYFE (SEQ ID NO: 515) (MM), wherein X₁ is absent or any amino acid, wherein X₂ is D, K, or R, and wherein the linker consists of 1 to 20 amino acids. In some aspects, the isolated polypeptide comprises an amino acid sequence ALTTVX-linker-ASHYFE (SEQ ID NO: 508) (MM), wherein X is D, K, or R, and wherein the N-terminal alanine residue is optionally absent or optionally substituted by any other amino acid. In some aspects, the linker consists of 1 to 20 amino acids. In some aspects, the N-terminal alanine residue is substituted by lysine. In some aspects, the linker consists of 1, 2, 3, 4, 5, 6, 7, 8, 9, 10, 11, 12, 13, 14, 15, 16, 17, 18, 19, or 20 amino acids. In some aspects, the isolated polypeptide comprises amino acid sequence comprises ALTTVDGGGGSASHYFE (SEQ ID NO: 512), ALTTVDGGGGSASHYFER (SEQ ID NO: 236), ALTTVDGGGGSASHYFEK (SEQ ID NO: 237), ALTTVKGGGGSASHYFE (SEQ ID NO: 513), ALTTVKGGGGSASHYFER (SEQ ID NO: 238), ALTTVKGGGGSASHYFEK (SEQID NO: 239), ALTTVRGGGGSASHYFE (SEQ ID NO: 514), ALTTVRGGGGSASHYFER (SEQID NO: 240), or ALTTVRGGGGSASHYFEK (SEQ ID NO: 241), or wherein the N-terminal alanine residue in each sequence is optionally absent or substituted by any other amino acid. In some aspects, the isolated polypeptide comprises amino acid sequence comprises a sequence selected from SQKLTTVDGGGGSASHYFERHLE (SEQ ID NO: 730), SQKLTTVRGGGGSASHYFERHLE (SEQ ID NO: 731), SQALTTVRGGGGSASHYFERHLE (SEQ ID NO: 732), or SQALTTVDGGGGSASHYFERHLE (SEQ ID NO: 733). In accordance with the present disclosure, the amino acid sequence is a masking moiety that inhibits binding of the cytokine with its receptor.

In one aspect, the present disclosure includes an isolated polypeptide further comprising a cytokine. In some aspects, the isolated polypeptide is disposed in a complex comprising two or more polypeptides, and wherein the complex comprises a cytokine. In one aspect, the present disclosure includes a complex comprising a polypeptide comprising a cytokine complexed with an isolated polypeptide of the present disclosure. In some aspects, the cytokine is disposed in a polypeptide that is complexed with the isolated polypeptide. In some aspects, the cytokine is a cytokine that binds IL2/IL15 receptor beta and/or IL2/IL15 receptor gamma. In some aspects, the cytokine is a cytokine that binds to IL-15Rα. In some aspects, the cytokine is a cytokine that binds to IL-2Rα.

In one aspect, the present disclosure includes an activatable cytokine construct (ACC) comprising a cytokine polypeptide (CP), a cleavable moiety (CM), and an isolated polypeptide (MM) of the present disclosure, wherein the MM is coupled to the CP via the CM and inhibits the binding of CP to its receptor.

In one aspect, the present disclosure includes an ACC comprising a first monomer construct and a second monomer construct, wherein the first monomer construct comprises a first cytokine polypeptide (CP1), a first cleavable moiety (CM1), a first dimerization domain (DD1) coupled to the CP1 via the CM1, and a first masking moiety (MM1), the second monomer construct comprises a second cytokine polypeptide (CP2), a second cleavable moiety (CM2), a second dimerization domain (DD2) coupled to the CP2 via the CM2, and a second masking moiety (MM2), the DD1 and DD2 bind each other thereby forming a dimer of the first and second monomer constructs, and the MM1 and/or the MM2 comprises an isolated polypeptide of the present disclosure.

In one aspect, the present disclosure includes an ACC comprising a first monomer construct and a second monomer construct, wherein the first monomer construct comprises a first cytokine polypeptide (CP1), a first dimerization domain (DD1), and a first masking moiety (MM1), the second monomer construct comprises a second cytokine polypeptide (CP2), a first cleavable moiety (CM1), a second dimerization domain (DD2) coupled to the CP2 via the CM1, and a second masking moiety (MM2), the MM1 and/or the MM2 is an isolated polypeptide of the present disclosure, and the DD1 and DD2 bind each other thereby forming a dimer of the first and second monomer constructs.

In one aspect, the present disclosure includes an ACC comprising a first monomer construct and a second monomer construct, wherein the first monomer construct comprises a first cytokine polypeptide (CP1), a first dimerization domain (DD1), and a first masking moiety (MM1), the second monomer construct comprises a second cytokine polypeptide (CP2), a second dimerization domain (DD2), and a second masking moiety (MM2), the CP1 and/or the CP2 comprises an amino acid sequence that functions as a substrate for a protease, and the DD1 and/or DD2 is coupled to the CP1 and/or CP2 via the amino acid sequence, the MM1 and/or the MM2 is an isolated polypeptide of the present disclosure, and the DD1 and DD2 bind each other thereby forming a dimer of the first and second monomer constructs.

In one aspect, the present disclosure includes an ACC comprising a first monomer construct and a second monomer construct, wherein the first monomer construct comprises a cytokine polypeptide (CP), a first dimerization domain (DD1), a first cleavable moiety (CM1), a second cleavable moiety (CM2), and an isolated polypeptide or masking moiety (MM) of the present disclosure, wherein the isolated peptide or MM is coupled to the CP via the CM1, and the DD1 is coupled to the CP via the CM2, the second monomer construct comprises an agonist of the CP, a third cleavable moiety (CM3), a second dimerization domain (DD2) coupled to the agonist via the CM3, and the DD1 and DD2 bind each other thereby forming a dimer of the first and second monomer constructs.

In one aspect, the present disclosure includes a polynucleotide encoding an isolated polypeptide of the present disclosure or a monomer construct of the present disclosure. The present disclosure also includes vectors, host cells, compositions, methods of manufacturing, and methods of treatment according to the following disclosures.

### BRIEF DESCRIPTION OF DRAWINGS

FIGS. 1A-1B show complexes of IL-15 and its receptors (FIG. 1A, left) or IL-2 (FIG. 1B, left), and exemplary MMs that bind to IL-15 (FIG. 1A, right) or IL-2 (FIG. 1B, right) and interrupt the binding between the interleukins and their receptors.

FIG. 2 is a schematic of an illustrative activatable cytokine construct comprising, from N-terminus to C-terminus: (1) a first monomer construct 110 having optionally a MM1 119, optionally a CM3 117, a CP1 115, a CM1 113, and a DD1 111, and; (2) a second monomer construct 120 having optionally a MM2 129, optionally a CM4 127, a CP2 125, a CM2 123, and a DD2 121; and (3) one or more covalent or non-covalent bonds (←→) bonding the first monomer construct 110 to the second monomer construct 120. The ACC may further comprise one or more of the optional linkers 112, 114, 116, 118, 122, 124, 126, and 128 between the components. In one example, DD1 111 and DD2 121 are the same. In another example, DD1 111 and DD2 121 are different.

FIGS. 3A-3E schematically show additional examples of ACCs. Exemplary ACCs having an MM (e.g., beta peptide), CMs (“substrates”), a CP, for example IL-15 (FIG. 3A), and a DD1 and a DD2 (Fc) in FIG. 3B. Exemplary ACCs with a cytokine agonist, e.g., Sushi domain, and an optional histidine tag (“His tag”) (FIGS. 3C-3E).

FIG. 4 schematically shows an embodiment of an ACC denoting its Linking Region (LR).

FIG. 5 shows a schematic of the structure of an exemplary ACC ProC2970 (top left), the tertiary structure of a monomer construct comprising an interleukin, a cleavable moiety, and a MM (top right), and the tertiary structure of the monomer construct in complex with its receptor (alpha, beta, and gamma chains of the receptor depicted) (bottom).

FIG. 6 shows results of electrophoresis testing the cleavage of exemplary ACC ProC2970 by uPA.

FIG. 7 shows the masking efficiency of the masking moiety on exemplary ACC ProC2970 tested by a reporter assay and compared to ProC1879.

FIG. 8 shows the activity of exemplary ACC ProC2970 on PMBC proliferation and compared to ProC1879.

FIGS. 9A-9E show activation of the ACCs. FIGS. 9A-9B show electrophoresis of ACCs before and after uPA cleavage. FIGS. 9C-9E show EC50 of ACCs in an HEK-Blue reporter assays decreases after uPA-mediated activation.

FIG. 10 shows a schematic of the structures of ACCs without MMs (top row) and exemplary ACC constructs having MMs (bottom row).

### DETAILED DESCRIPTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or aspects so described.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and Figs., and from the claims.

The term “a” and “an” refers to one or more (i.e., at least one) of the grammatical object of the article. By way of example, “a cell” encompasses one or more cells.

As used herein, the terms “about” and “approximately,” when used to modify an amount specified in a numeric value or range, indicate that the numeric value as well as reasonable deviations from the value known to the skilled person in the art. For example ±20%, ±10%, or ±5%, are within the intended meaning of the recited value where appropriate.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” or “consisting of” would find direct support due to this definition for any elements disclosed throughout this disclosure. Based on this definition, any element disclosed herein or incorporated by reference may be included in or excluded from the claimed invention.

As used herein, a plurality of compounds, elements, or steps may be presented in a common list for convenience. However, these lists should be construed as though each member of the list is individually identified as a separate and unique member. Thus, no individual member of such list should be construed as a defacto equivalent of any other member of the same list solely based on their presentation in a common group without indications to the contrary.

Furthermore, certain molecules, constructs, compositions, elements, moieties, excipients, disorders, conditions, properties, steps, or the like may be discussed in the context of one specific embodiment or aspect or in a separate paragraph or section of this disclosure. It is understood that this is merely for convenience and brevity, and any such disclosure is equally applicable to and intended to be combined with any other embodiments or aspects found anywhere in the present disclosure and claims, which all form the application and claimed invention at the filing date. For example, a list of constructs, molecules, method steps, kits, or compositions described with respect to a construct, composition, or method is intended to and does find direct support for embodiments related to constructs, compositions, formulations, and methods described in any other part of this disclosure, even if those method steps, active agents, kits, or compositions are not re-listed in the context or section of that embodiment or aspect.

The terms “cleavable moiety” and “CM” are used interchangeably herein to refer to a polypeptide, the amino acid sequence of which comprises a substrate for a sequence-specific protease. Cleavable moieties that are suitable for use in the ACCs herein include any of the protease substrates that are known the art. Exemplary cleavable moieties are described in more detail below.

The terms “masking moiety” or “MM” are used interchangeably herein to refer to a peptide or protein that reduces or inhibits one or more activities of a cytokine polypeptide. In some embodiments, when positioned proximal to a cytokine polypeptide, a MM interferes with binding of the cytokine polypeptide to its binding partner (e.g., its receptor). In some embodiments, the MM is an amino acid sequence of less than 50 amino acids including any number of amino acids or range of amino acids within 1 to 50. In some embodiments, the MM is no more than 40 amino acids in length. In preferred embodiments, the MM is no more than 20 amino acids in length. In some embodiments, the MM is no more than 19, 18, 17, 16, or 15 amino acids in length. In some aspects, the MM is at least 1, 2, 3, 4 amino acids. In some aspects, the MM is 5, 6, 7, 8, 9, 10, 11, 12, 13, or 14 amino acids.

As used herein, the term “masking efficiency” refers to the activity (e.g., EC50) of the uncleaved ACC divided by the activity of a control interleukin, wherein the control interleukin may be either cleavage product of the ACC or the cytokine used as the CP of the ACC. An ACC having a reduced level of at least one interleukin activity has a masking efficiency that is greater than 10. In some embodiments, the ACCs described herein have a masking efficiency that is greater than 10, greater than 100, greater than 1000, or greater than 5000. In some embodiments, the ACC has a masking efficiency that is about 10 to about 100, or about 10 to about 200, or about 50 to about 150, or about 50 to about 80, as measured by the ratio of the EC50 of the uncleaved ACC to the EC50 of the cleavage product of the ACC in IL-2/IL-15 responsive HEK293 cells.

As used herein, the term “subsequence” means that the moiety does not include the full length amino acid sequence of the cytokine receptor sequence and instead has fewer than all of the amino acids in the amino acid sequence of the cytokine receptor sequence. Accordingly, as used herein, a subunit, monomer, construct, polypeptide, or amino acid sequence that is “encoded by” a subsequence does not include the full length amino acid sequence of the cytokine receptor sequence and instead the subunit, monomer, construct, polypeptide, or amino acid sequence has fewer than all of the amino acids in the amino acid sequence of the cytokine receptor sequence.

As used herein “continuous” means two or more adjacent amino acids in the subsequence the same order from the N- to C-terminal direction.

The term “activatable” when used in reference to a cytokine construct, refers to a cytokine construct that exhibits a first level of one or more activities, whereupon exposure to a condition that causes cleavage of one or more cleavable moieties results in the generation of a cytokine construct that exhibits a second level of the one or more activities, where the second level of activity is greater than the first level of activity. Non-limiting examples of activities include any of the exemplary activities of a cytokine described herein or known in the art.

The term “mature cytokine polypeptide” refers herein to a cytokine polypeptide that lacks a signal sequence. A cytokine polypeptide (e.g., an interleukin polypeptide) may be a mature cytokine polypeptide or a cytokine polypeptide with a signal peptide. Thus, the ACCs of the present disclosure may include a mature cytokine polypeptide sequence in some aspects. In some aspects, the ACCs of the present disclosure may include a mature cytokine polypeptide sequence and, additionally, a signal sequence. In some aspects, the ACCs of the present disclosure may include sequences disclosed herein, including or lacking the signal sequences recited herein.

The terms “dimerization domain” and “DD” are used interchangeably herein to refer to one member of a pair of dimerization domains, wherein each member of the pair is capable of binding to the other via one or more covalent or non-covalent interactions. The first DD and the second DD may be the same or different. Exemplary DDs suitable for use as DD1 and or DD2 are described in more detail herein below.

As used herein, a polypeptide, such as a cytokine or an Fc domain, may be a wild-type polypeptide (e.g., a naturally-existing polypeptide) or a variant of the wild-type polypeptide. A variant may be a polypeptide modified by substitution, insertion, deletion and/or addition of one or more amino acids of the wild-type polypeptide, provided that the variant retains the basic function or activity of the wild-type polypeptide. In some examples, a variant may have altered (e.g., increased or decreased) function or activity compared with the wild-type polypeptide. In some aspects, the variant may be a functional fragment of the wild-type polypeptide. The term “functional fragment” means that the sequence of the polypeptide (e.g., cytokine) may include fewer amino acids than the full-length polypeptide sequence, but sufficient polypeptide chain length to confer activity (e.g., cytokine activity). Conservative amino acid substitutions are ones in which the amino acid residue is replaced with an amino acid residue having a similar side chain. Families of amino acid residues having similar side chains have been defined in the art. These families include: amino acids with acidic side chains (e.g., aspartate and glutamate), amino acids with basic side chains (e.g., lysine, arginine, and histidine), non-polar amino acids (e.g., alanine, valine, leucine, isoleucine, proline, phenylalanine, methionine, and tryptophan), uncharged polar amino acids (e.g., glycine, asparagine, glutamine, cysteine, serine, threonine and tyrosine), hydrophilic amino acids (e.g., arginine, asparagine, aspartate, glutamine, glutamate, histidine, lysine, serine, and threonine), hydrophobic amino acids (e.g., alanine, cysteine, isoleucine, leucine, methionine, phenylalanine, proline, tryptophan, tyrosine, and valine). Other families of amino acids include: aliphatic-hydroxy amino acids (e.g., serine and threonine), amide family (e.g., asparagine and glutamine), aliphatic family (e.g., alanine, valine, leucine and isoleucine), aromatic family (e.g., phenylalanine, tryptophan, and tyrosine).

The term “at least [a certain]% identical to” in the context of two or more nucleic acid or amino acid sequences means that the two or more sequences have nucleotides or amino acid residues in common in the given percent when compared and aligned for maximum correspondence over a comparison window or designated sequences of nucleic acids or amino acids (i.e. the sequences have at least 90 percent (%) identity). Percent identity of nucleic acid or amino acid sequences can be measured using a BLAST sequence comparison algorithm with default parameters, or by manual alignment and visual inspection (see e.g. blast.ncbi.nlm.nih.gov/Blast.cgi). Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:
100 times the fraction X/Y
where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

As used herein, “isolated polypeptide” means a polypeptide of cDNA, recombinant RNA, or synthetic origin, or some combination thereof, which by virtue of its origin, or source of derivation, the “isolated polypeptide” is substantially free of endogenously expressed constituents of a host cell, such as a mammalian cell or, in the case of a cell-free expression system, substantially free of cell-free expression reagents and does not occur in nature. The isolated polypeptide may be substantially free of endogenously expressed constituents of a host cell or substantially free of cell-free expression reagents using conventional separation techniques, for example chromatography. According to aspects of the present disclosure, the isolated polypeptide may be disposed in a complex comprising two or more polypeptides including wherein the complex comprises a cytokine. Unless otherwise specified, a “nucleic acid sequence encoding a protein” includes all nucleotide sequences that are degenerate versions of each other and thus encode the same amino acid sequence.

The term “N-terminally” when referring to a position of a first domain or sequence relative to a second domain or sequence in a polypeptide primary amino acid sequence means that the first domain or sequence is located closer to the N-terminus of the polypeptide primary amino acid sequence than the second domain or sequence. In some embodiments, there may be additional sequences and/or domains between the first domain or sequence and the second domain or sequence.

The term “C-terminally” when referring to a position of a first domain or sequence relative to a second domain or sequence in a polypeptide primary amino acid sequence means that the first domain or sequence is located closer to the C-terminus of the polypeptide primary amino acid sequence than the second domain or sequence. In some embodiments, there may be additional sequences and/or domains between the first domain or sequence and the second domain or sequence.

The term “exogenous” refers to any material introduced from or originating from outside a cell, a tissue, or an organism that is not produced by or does not originate from the same cell, tissue, or organism in which it is being introduced.

The term “transduced,” “transfected,” or “transformed” refers to a process by which an exogenous nucleic acid is introduced or transferred into a cell. A “transduced,” “transfected,” or “transformed” cell (e.g., mammalian cell) is one that has been transduced, transfected, or transformed with exogenous nucleic acid (e.g., a vector) that includes an exogenous nucleic acid encoding any of the activatable cytokine constructs described herein.

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

As used herein the phrase “specifically binds” means that the ACC binds to its receptor or target and does not react with other polypeptides, or binds at much lower affinity, e.g., about or greater than 10⁻⁶ M.

The term “treatment” refers to ameliorating at least one symptom of a disorder. In some embodiments, the disorder being treated is a cancer. In some embodiments, the disorder being treated is an autoimmune disorder. In some embodiments, the disorder being treated is an inflammatory disorder.

### Polypeptides Comprising Novel Masking Moieties

Provided herein are isolated polypeptides comprising an amino acid sequence that can be used as a masking moiety in an activatable cytokine construct.

In some aspects, the present disclosure provides isolated polypeptides comprising masking moieties (MMs) that interrupt binding between interleukins and its binding partner.

In some aspects, the present disclosure provides activatable cytokine constructs (ACCs) in various formats that integrate the MMs.

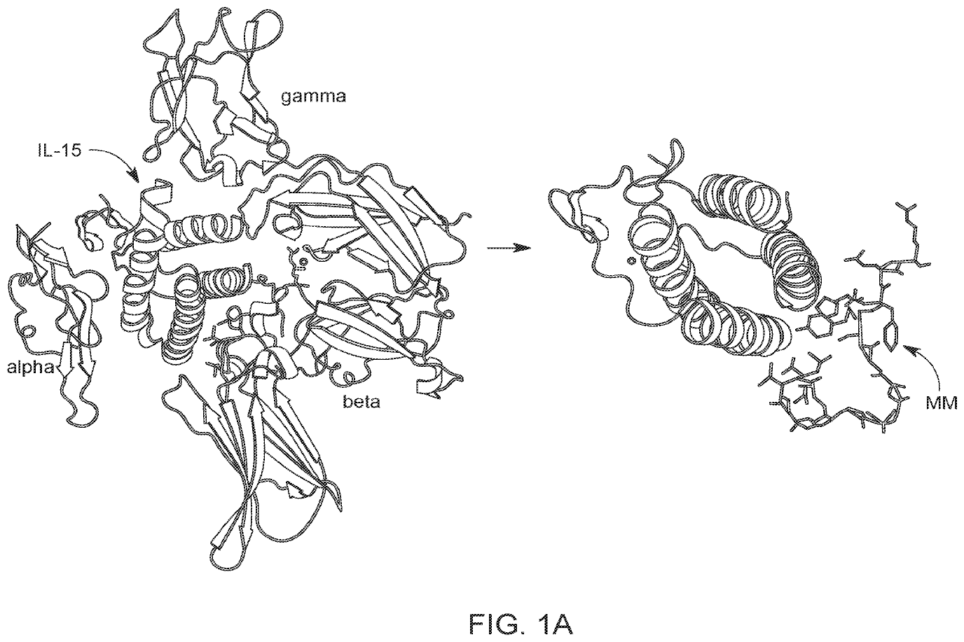

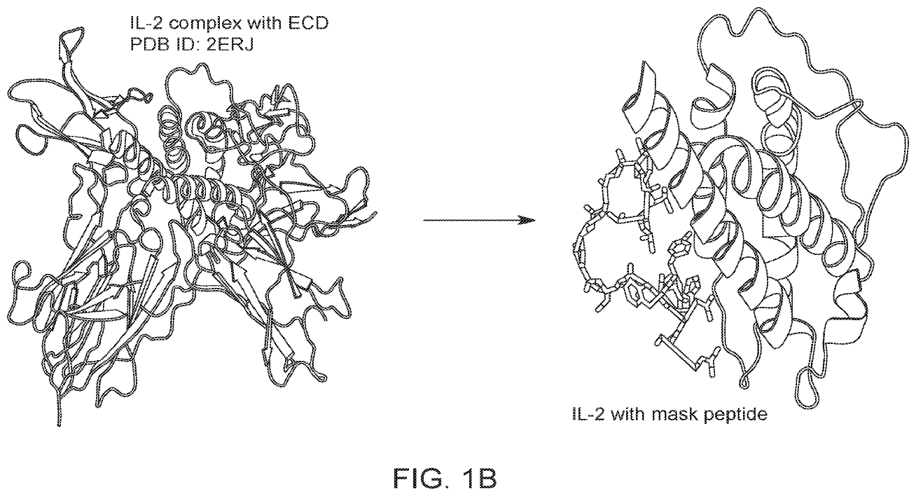

In some aspects, the present disclosure provides activatable cytokine constructs (ACCs) that exhibit a reduced level of at least one activity of the corresponding cytokine, but which, after exposure to an activation condition, yield a cytokine product having substantially restored activity. In some embodiments, the ACCs comprise a cytokine polypeptide (CP), a cleavable moiety (CM), and a masking moiety (MM) according to the present disclosures. In some embodiments, the MM disrupts the interaction between a CP and its binding partner (e.g., its receptor). In some embodiments, the MM binds to IL-15 (FIG. 1A) or IL-2 (FIG. 1B), and interrupts the binding between the interleukins and their receptors.

The inventors have surprisingly found that ACCs comprising the MMs disclosed herein have improved characteristics, such as higher masking efficiency, compared to a counterpart ACC not comprising such MMs.

ACCs of the present disclosure may selectively activate upon exposure to diseased tissue, and not in normal tissue. Following activation of the ACC upon cleavage of the cleavable moieties, cytokine activity is restored indicating that released masking moieties do not appear to remain bound to the cytokine after cleavage and do not interfere or compete with the cytokine for binding to its target. As such, the ACCs have the potential for conferring the benefit of a cytokine-based therapy, with potentially less of the toxicity associated with certain cytokine-based therapies and improved pharmacokinetics.

Also provided herein are related intermediates, compositions, kits, nucleic acids, and recombinant cells, as well as related methods, including methods of using and methods of producing and delivering any of the ACCs described herein.

In one aspect, the present disclosure provides polypeptides (e.g., isolated polypeptides) comprising one or more masking moieties (MMs), where the MM comprises the sequence of ALTTVX-linker-ASHYFE (SEQ ID NO: 508), where X is D, K, or R, and wherein the N-terminal alanine residue is optionally absent or optionally substituted by any other amino acid. The MM sequences are linked with one or more linker sequences, e.g., flexible linkers, linkers comprising Gly, Ser, Thr, Asn, Pro, such as those disclosed below, linkers designed to impart specific structures, and the like.

In some embodiments, the linker consists of 1 to 22 amino acids. In one example, the linker consists of 1 amino acid. In another example, the linker consists of 2 amino acids. In another example, the linker consists of 3 amino acids. In another example, the linker consists of 4 amino acids. In another example, the linker consists of 5 amino acids. In another example, the linker consists of 6 amino acids. In another example, the linker consists of 7 amino acids. In another example, the linker consists of 8 amino acids. In another example, the linker consists of 9 amino acids. In another example, the linker consists of 10 amino acids. In another example, the linker consists of 11 amino acids. In another example, the linker consists of 12 amino acids. In another example, the linker consists of 13 amino acids. In another example, the linker consists of 14 amino acids. In another example, the linker consists of 15 amino acids. In another example, the linker consists of 16 amino acids. In another example, the linker consists of 17 amino acids. In another example, the linker consists of 18 amino acids. In another example, the linker consists of 19 amino acids. In another example, the linker consists of 20 amino acids. In another example, the linker consists of 21 amino acids. In another example, the linker consists of 22 amino acids. In some examples, the linker consists of 21-53 amino acids, e.g., 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 amino acids.

Examples of linkers include sequences described below, e.g., SEQ ID NOs: 2 and 210-235, 245, or 250. In one example, the linker is GGGGS (SEQ ID NO: 216).

In some embodiments, the MM comprises the sequence of ALTTVD-linker-ASHYFE (SEQ ID NO: 509) or ALTTVD-linker-ASHYFER (SEQ ID NO: 242) or ALTTVD-linker-ASHYFEK (SEQ ID NO: 243), or wherein the N-terminal alanine residue is optionally absent or substituted by any other amino acid. In some embodiments, the MM comprises the sequence of ALTTVK-linker-ASHYFE (SEQ ID NO: 510) or ALTTVK-linker-ASHYFER (SEQ ID NO: 244) or ALTTVK-linker-ASHYFEK (SEQ ID NO: 246), or wherein the N-terminal alanine residue is optionally absent or substituted by any other amino acid. In some embodiments, the MM comprises the sequence of ALTTVR-linker-ASHYFE (SEQ ID NO: 511) or ALTTVR-linker-ASHYFER (SEQ ID NO: 247) or ALTTVR-linker-ASHYFEK (SEQ ID NO: 248), or wherein the N-terminal alanine residue is optionally absent or substituted by any other amino acid. In some aspects, the N-terminal alanine residue is substituted by lysine. In some aspects, the N-terminus, the C-terminus, or both is extended by adding 1, 2, 3, 4, 5, 6, 7, 8, 9, 10, 11, 12, 13, 14, 15, 16, 17, 18, 19, or 20 amino acids. In certain aspects, the isolated polypeptide comprises or consists of a sequence selected from SQKLTTVDGGGGSASHYFERHLE (SEQ ID NO: 730), SQKLTTVRGGGGSASHYFERHLE (SEQ ID NO: 731), SQALTTVRGGGGSASHYFERHLE (SEQ ID NO: 732), or SQALTTVDGGGGSASHYFERHLE (SEQ ID NO: 733).

In some embodiments, the MM consists of the sequence of ALTTVD-linker-ASHYFER (SEQ ID NO: 242) or ALTTVD-linker-ASHYFE(R/K) (SEQ ID NO: 502), or wherein the N-terminal alanine residue is optionally absent or substituted by any other amino acid. In some embodiments, the MM consists of the sequence of ALTTVK-linker-ASHYFE (SEQ ID NO: 510) or ALTTVK-linker-ASHYFE(R/K) (SEQ ID NO: 503), or wherein the N-terminal alanine residue is optionally absent or substituted by any other amino acid. In some embodiments, the MM consists of the sequence of ALTTVR-linker-ASHYFE (SEQ ID NO: 511) or ALTTVR-linker-ASHYFE(R/K) (SEQ ID NO: 504), or wherein the N-terminal alanine residue is optionally absent or substituted by any other amino acid. In some aspects, the N-terminal alanine residue is substituted by lysine.

In one example, the MM comprises the sequence of ALTTVDGGGGSASHYFE (SEQ ID NO: 512) or ALTTVDGGGGSASHYFE(R/K) (SEQ ID NO: 505), or wherein the N-terminal alanine residue is optionally absent or substituted by any other amino acid. In another example, the MM comprises the sequence of ALTTVKGGGGSASHYFE (SEQ ID NO: 513) or ALTTVKGGGGSASHYFE(R/K) (SEQ ID NO: 506), or wherein the N-terminal alanine residue is optionally absent or substituted by any other amino acid. In another example, the MM comprises the sequence of ALTTVRGGGGSASHYFE (SEQ ID NO 514) or ALTTVRGGGGSASHYFE(R/K) (SEQ ID NO: 507), or wherein the N-terminal alanine residue is optionally absent or substituted by any other amino acid. In some aspects, the N-terminus, the C-terminus, or both is extended by adding 1, 2, 3, 4, 5, 6, 7, 8, 9, 10, 11, 12, 13, 14, 15, 16, 17, 18, 19, or 20 amino acids. In certain aspects, the isolated polypeptide comprises a sequence selected from SQKLTTVDGGGGSASHYFERHLE (SEQ ID NO: 730), SQKLTTVRGGGGSASHYFERHLE (SEQ ID NO: 731), SQALTTVRGGGGSASHYFERHLE (SEQ ID NO: 732), or SQALTTVDGGGGSASHYFERHLE (SEQ ID NO: 733).

In one example, the MM consists of the sequence of ALTTVDGGGGSASHYFE (SEQ ID NO: 512) or ALTTVDGGGGSASHYFE(R/K) (SEQ ID NO: 505), or wherein the N-terminal alanine residue is optionally absent or substituted by any other amino acid. In another example, the MM consists of the sequence of ALTTVKGGGGSASHYFE (SEQ ID NO: 513) or ALTTVKGGGGSASHYFE(R/K) (SEQ ID NO: 506), or wherein the N-terminal alanine residue is optionally absent or substituted by any other amino acid. In another example, the MM consists of the sequence of ALTTVRGGGGSASHYFE (SEQ ID NO: 514) or ALTTVRGGGGSASHYFE(R/K) (SEQ ID NO: 507), or wherein the N-terminal alanine residue is optionally absent or substituted by any other amino acid. In some aspects, the N-terminus, the C-terminus, or both is extended by adding 1, 2, 3, 4, 5, 6, 7, 8, 9, 10, 11, 12, 13, 14, 15, 16, 17, 18, 19, or 20 amino acids. In certain aspects, the isolated polypeptide comprises a sequence selected from SQKLTTVDGGGGSASHYFERHLE (SEQ ID NO: 730), SQKLTTVRGGGGSASHYFERHLE (SEQ ID NO: 731), SQALTTVRGGGGSASHYFERHLE (SEQ ID NO: 732), or SQALTTVDGGGGSASHYFERHLE (SEQ ID NO: 733).

The linkers in the MM may comprise any one or more amino acids and any combination of amino acid sequences. In some aspects, the linker is a flexible linker. In some aspects, the linker is designed to be impart a desired two-dimensional and/or three-dimensional structure to the MM.

In some embodiments, a masking moiety (MM) “masks” or reduces or otherwise inhibits the activity of a cytokine polypeptide. In some embodiments, a MM masks, reduces, or otherwise inhibits the binding of a cytokine polypeptide to its receptor. In some embodiments, the coupling or modifying of a cytokine polypeptide with a MM inhibits the ability of the cytokine polypeptide to specifically bind its receptor by means of inhibition known in the art (e.g., structural change and competition for receptor binding). In some embodiments, the coupling or modifying of a cytokine polypeptide with a MM affects a structural change that reduces or inhibits the ability of the protein to specifically bind its receptor. In some embodiments, the coupling or modifying of a cytokine polypeptide with a MM sterically blocks, reduces or inhibits the ability of the cytokine polypeptide to specifically bind its receptor.

### Activatable Cytokine Constructs

In one aspect, the present disclosure provides activatable cytokine constructs (ACCs) that comprises a cytokine polypeptide (CP), e.g., an interleukin polypeptide, a cleavable moiety (CM), and an MM described herein coupled with the CP via the CM.

In some examples, the ACC comprises, from its N-terminus to C-terminus, cytokine polypeptide-CM-MM, or MM-CM-cytokine polypeptide. As used herein and unless otherwise stated, each dash (-) between the ACC components represents either a direct linkage or linkage via, e.g., one or more linkers.

In some embodiments, the ACC is characterized by having a level of cytokine activity that is reduced by at least 1000-, 2000-, 3000-, 4000-, 5000-, or 6000-fold as compared to the corresponding recombinant wild-type cytokine. For example the ACC is characterized by having an EC50 that is at least 1000-, 2000-, 3000-, 4000-, 5000-, or 6000-fold greater than the EC50 of recombinant wild type IL-15, as measured in IL-2/IL15-responsive HEK293 cells.

In some embodiments, the ACC further comprises an agonist of the CP, such as a Sushi domain. For example, an ACC may comprise a MM described herein, a CM, a CP (e.g., such as IL-15 or a mutant thereof), and an agonist of the CP (e.g., a Sushi domain). In some examples, the agonist (e.g., Sushi domain) is coupled to the CP via a linker.

As used herein the term “Sushi domain” has its general meaning in the art and refers to a domain beginning at the first cysteine residue (C1) after the signal peptide of IL-15Rα, and ending at the fourth cysteine residue (C4) after said signal peptide. Said sushi domain corresponding to a portion of the extracellular region of IL-15Rα is necessary for its binding to IL-15 (Wei et al., J. Immunol., vol. 167(1), p: 277-282, 2001, incorporated herein by reference in its entirety). In one example, the Sushi domain comprises the sequence of SEQ ID NO: 520. In another example, the Sushi domain comprises a functional fragment of the sequence of SEQ ID NO: 520. Said sushi domain of IL-15Rα or derivatives thereof has at least 10% of the binding activity of the sushi domain of human IL-15Rα to human interleukin-15, e.g., at least 25% and more preferably at least 50%. Said binding activity can be simply determined by the method disclosed in Wei et al. 2001 mentioned above.

In some aspects, the sushi domain is covalently linked to the interleukin polypeptide, the MM, the DD1, or the DD2. In some aspects, the covalent linkage is a non-alpha-carbon covalent bond, e.g., an isopeptide bond. In some embodiments, the isopeptide bond is between a lysine and a glutamate or aspartate residue. In some embodiments, the non-alpha-carbon covalent bond is between functional groups substituted into an alpha-carbon in the MM and the cytokine. In some embodiments, the isopeptide bond is between the gamma-carboxyamide group of glutamine and epsilon-amino group of lysine sidechains. In some embodiments, the non-alpha-carbon covalent bond is an ester bond between threonine and glutamine. In some embodiments, the non-alpha-carbon covalent bond is a thioester bond between cysteine and glutamine. In some embodiments, the non-alpha-carbon covalent bond is a thioether bond between cysteine and tyrosine. In some embodiments, the non-alpha-carbon covalent bond is formed by crosslinking between histidine and tyrosine (e.g., this type of histidine-tyrosine crosslinking is known to exist in cytochrome c oxidase enzymes). In some embodiments, the non-alpha-carbon covalent bond is a nitrogen-oxygen-sulfur (NOS) bond formed between lysine and cysteine. In some embodiments, the non-alpha-carbon covalent bond is a disulfide bond.

In an ACC, a MM may be coupled to a cytokine polypeptide by a CM and optionally one or more linkers, as described in more detail herein. In some embodiments, when an ACC is not activated, the MM prevents the cytokine polypeptide from binding to its receptor; but when the ACC is activated (when the CM between the MM and the cytokine polypeptide is cleaved by a protease), the MM does not substantially or significantly interfere with the cytokine polypeptide's binding to the receptor.

In an ACC, a MM may be coupled to the cytokine polypeptide either directly or indirectly, e.g., via one or more linkers. Alternatively, a MM may be coupled, either directly or indirectly, to a component of the ACC that is not the cytokine polypeptide. For example, the MM may be coupled, either directly or indirectly, to a different cytokine polypeptide. In another example, the MM may be coupled, either directly or indirectly, with a DD. In either case, in the tertiary or quaternary structure of the activatable structure, the MM may be in a position (e.g., proximal to the cytokine polypeptide to be masked) that allows the MM to mask the cytokine polypeptide.

In some embodiments, the ACC further comprises an agonist of the CP, such as a Sushi domain as described below. For example, an ACC may comprise a MM described herein, a CM, a CP (e.g., such as IL-15 or a mutant thereof), and an agonist of the CP (e.g., a Sushi domain). In some examples, the agonist (e.g., Sushi domain) is coupled to the CP via a linker.

### Interleukin Polypeptide

In some embodiments, the CP is an interleukin polypeptide. Examples of the interleukin polypeptide in the ACC herein may include IL-1α, IL-1β, IL-1RA, IL-18, IL-2, IL-4, IL-7, IL-9, IL-13, IL-15, IL-3, IL-4, IL-5, IL-6, IL-7, IL-9, IL-11, IL-12, IL-10, IL-20, IL-21 IL-14, IL-15, IL-16, and IL-17, and IL-21.

In some examples, the CP is IL-15. For example, the CP may comprise SEQ ID NO: 348, 129, or 130, or a functional fragment thereof. In some examples, the CP may comprise a sequence at least 85%, 90%, or 95% identical to SEQ ID NO: 348, 129, or 130.

In some embodiments, the interleukin polypeptide is a wild-type IL-15. In some embodiments, the interleukin polypeptide is a wild-type human IL-15. In some embodiments, the interleukin polypeptide is a mutant IL-15. In some embodiments, the interleukin polypeptide is a mutant human IL-15. In some embodiments, the interleukin polypeptide is at least 85%, 90%, 95%, 99%, or 100% identical to SEQ ID NO: 348. In some embodiments, the interleukin polypeptide is at least 85% identical to IL-15 (SEQ ID NO: 348), where the amino acid at position 45 of the interleukin polypeptide is not leucine. In some examples, the interleukin polypeptide is at least 90% identical to SEQ ID NO: 348, where the amino acid at position 45 of the interleukin polypeptide is not leucine. In some examples, the interleukin polypeptide is at least 95% identical to SEQ ID NO: 348, where the amino acid at position 45 of the interleukin polypeptide is not leucine. In some examples, the interleukin polypeptide is at least 99% identical to SEQ ID NO: 348, where the amino acid at position 45 of the interleukin polypeptide is not leucine. The positions of mutations are relative to a reference sequence. Thus, for example, where the mutation is at position 45, it is relative to the reference sequence. For example, where the reference sequence is:

(SEQ ID NO: 348)
NWVNVISDLKKIEDLIQSMHIDATLYTESDVHPSCKVTAMK
CFLLELQVISLESGDASIHDTVENLIILANNSLSSNGNVTE
SGCKECEELEEKNIKEFLQSFVHIVQMFINTS,

amino acids at positions 45 and 52 are bolded.

In some examples, the amino acid at position corresponding to position 45 of SEQ ID NO: 348 in the interleukin polypeptide is aspartic acid. In some examples, the amino acid at position corresponding to position 45 of SEQ ID NO: 348 in the interleukin polypeptide is asparagine. In some examples, the amino acid at position corresponding to position 45 of SEQ ID NO: 348 in the interleukin polypeptide is threonine.

In some embodiments, the interleukin polypeptide is at least 85% identical to SEQ ID NO: 348, where the amino acid at position 52 of the interleukin polypeptide is not leucine. In some examples, the interleukin polypeptide is at least 90% identical to SEQ ID NO: 348, where the amino acid at position 52 of the interleukin polypeptide is not leucine. In some examples, the interleukin polypeptide is at least 95% identical to SEQ ID NO: 348, where the amino acid at position 52 of the interleukin polypeptide is not leucine. In some examples, the interleukin polypeptide is at least 99% identical to SEQ ID NO: 348, where the amino acid at position 52 of the interleukin polypeptide is not leucine.

In some examples, the amino acid at position corresponding to position 52 of SEQ ID NO: 348 in the interleukin polypeptide is aspartic acid. In some examples, the amino acid at position corresponding to position 52 of SEQ ID NO: 348 in the interleukin polypeptide is asparagine. In some examples, the amino acid at position corresponding to position 52 of SEQ ID NO: 348 in the interleukin polypeptide is threonine.

In some embodiments, the interleukin polypeptide is at least 85% identical to SEQ ID NO: 348, where the amino acid at position 45 of the interleukin polypeptide is not leucine, and the amino acid at position 52 is not leucine. In some examples, the interleukin polypeptide is at least 90% identical to SEQ ID NO: 348, where the amino acid at position 45 of the interleukin polypeptide is not leucine, and the amino acid at position 52 is not leucine. In some embodiments, the interleukin polypeptide is at least 95% identical to SEQ ID NO: 348, where the amino acid at position 45 of the interleukin polypeptide is not leucine, and the amino acid at position 52 is not leucine.

In some examples, the amino acids at positions corresponding to positions 45 and 52 of SEQ ID NO: 348 in the interleukin polypeptide are aspartic acid. In some examples, the amino acids at positions corresponding to positions 45 and 52 of SEQ ID NO: 348 in the interleukin polypeptide are asparagine. In some examples, the amino acids at positions corresponding to positions 45 and 52 of SEQ ID NO: 348 in the interleukin polypeptide are threonine.

In some examples, the amino acids at positions corresponding to positions 45 and 52 of SEQ ID NO: 348 in the interleukin polypeptide are aspartic acid, asparagine, or threonine.

In some embodiments, the interleukin polypeptide comprises any of SEQ ID NO: 402-422. In one example, the interleukin polypeptide comprises SEQ ID NO: 402. In another example, the interleukin polypeptide comprises SEQ ID NO: 403. In another example, the interleukin polypeptide comprises SEQ ID NO: 404. In another example, the interleukin polypeptide comprises SEQ ID NO: 405. In another example, the interleukin polypeptide comprises SEQ ID NO: 406. In another example, the interleukin polypeptide comprises SEQ ID NO: 407. In another example, the interleukin polypeptide comprises SEQ ID NO: 408. In another example, the interleukin polypeptide comprises SEQ ID NO: 409. In another example, the interleukin polypeptide comprises SEQ ID NO: 410. In another example, the interleukin polypeptide comprises SEQ ID NO: 411. In another example, the interleukin polypeptide comprises SEQ ID NO: 412. In another example, the interleukin polypeptide comprises SEQ ID NO: 413. In another example, the interleukin polypeptide comprises SEQ ID NO: 414. In another example, the interleukin polypeptide comprises SEQ ID NO: 415. In another example, the interleukin polypeptide comprises SEQ ID NO: 416. In another example, the interleukin polypeptide comprises SEQ ID NO: 417. In another example, the interleukin polypeptide comprises SEQ ID NO: 418. In another example, the interleukin polypeptide comprises SEQ ID NO: 419. In another example, the interleukin polypeptide comprises SEQ ID NO: 420. In another example, the interleukin polypeptide comprises SEQ ID NO: 421. In another example, the interleukin polypeptide comprises SEQ ID NO: 422.

In some embodiments, the interleukin polypeptide consists of any of SEQ ID NO: 402-422. In one example, the interleukin polypeptide consists of SEQ ID NO: 402. In another example, the interleukin polypeptide consists of SEQ ID NO: 403. In another example, the interleukin polypeptide consists of SEQ ID NO: 404. In another example, the interleukin polypeptide consists of SEQ ID NO: 405. In another example, the interleukin polypeptide consists of SEQ ID NO: 406. In another example, the interleukin polypeptide consists of SEQ ID NO: 407. In another example, the interleukin polypeptide consists of SEQ ID NO: 408. In another example, the interleukin polypeptide consists of SEQ ID NO: 409. In another example, the interleukin polypeptide consists of SEQ ID NO: 410. In another example, the interleukin polypeptide consists of SEQ ID NO: 411. In another example, the interleukin polypeptide consists of SEQ ID NO: 412. In another example, the interleukin polypeptide consists of SEQ ID NO: 413. In another example, the interleukin polypeptide consists of SEQ ID NO: 414. In another example, the interleukin polypeptide consists of SEQ ID NO: 415. In another example, the interleukin polypeptide consists of SEQ ID NO: 416. In another example, the interleukin polypeptide consists of SEQ ID NO: 417. In another example, the interleukin polypeptide consists of SEQ ID NO: 418. In another example, the interleukin polypeptide consists of SEQ ID NO: 419. In another example, the interleukin polypeptide consists of SEQ ID NO: 420. In another example, the interleukin polypeptide consists of SEQ ID NO: 421. In another example, the interleukin polypeptide consists of SEQ ID NO: 422.

In some examples, the CP is IL-2 or a functional fragment thereof. For example, the CP may comprise SEQ ID NO: 119 or 120, or a functional fragment thereof. In some examples, the CP may comprise a sequence at least 85%, 90%, or 95% identical to SEQ ID NO: 119 or 120.

In some examples, the CP is IL-4 or a functional fragment thereof. For example, the CP may comprise SEQ ID NO: 121 or 122, or a functional fragment thereof. In some examples, the CP may comprise a sequence at least 85%, 90%, or 95% identical to SEQ ID NO: 121 or 122.

In some examples, the CP is IL-7 or a functional fragment thereof. For example, the CP may comprise SEQ ID NO: 123 or 124, or a functional fragment thereof. In some examples, the CP may comprise a sequence at least 85%, 90%, or 95% identical to SEQ ID NO: 123 or 124.

In some examples, the CP is IL-9 or a functional fragment thereof. For example, the CP may comprise SEQ ID NO: 125 or 126, or a functional fragment thereof. In some examples, the CP may comprise a sequence at least 85%, 90%, or 95% identical to SEQ ID NO: 125 or 126.

In some examples, the CP is IL-21 or a functional fragment thereof. For example, the CP may comprise SEQ ID NO: 521 or 522, or a functional fragment thereof. In some examples, the CP may comprise a sequence at least 85%, 90%, or 95% identical to SEQ ID NO: 521 or 522.

ACC with Dimerization Domains

In some embodiments, an ACC herein is a dimer complex comprising a first monomer construct and a second monomer construct. Dimerization of the monomeric components is facilitated by a pair of dimerization domains. In one aspect, each monomer construct includes a cytokine polypeptide, a MM described herein, and a dimerization domain (DD).

In a specific embodiment, the present invention provides an ACC that includes a first monomer construct and a second monomer construct, wherein:
the first monomer construct comprises a first cytokine polypeptide (CP1), a first cleavable moiety (CM1), a first dimerization domain (DD1) coupled to the CP1 via the CM1, and a first masking moiety (MM1),
the second monomer construct comprises a second cytokine polypeptide (CP2), a second cleavable moiety (CM2), a second dimerization domain (DD2) coupled to the CP2 via the CM2, and a second masking moiety (MM2),
the DD1 and DD2 bind each other thereby forming a dimer of the first and second monomer constructs, and
the MM1 and/or the MM2 comprises the MM described herein.

In some embodiments, the ACC is characterized by having a reduced level of at least one CP1 and/or CP2 activity as compared to a control level of the at least one CP1 and/or CP2 activity.

In some embodiments, the first monomer construct comprises a third cleavable moiety (CM3) and the MM1 is coupled to the CP1 via the CM3. In some embodiments, the MM1 is coupled to the CP1 via the CM1. In some embodiments, the second monomer construct comprises a fourth cleavable moiety (CM4) and the MM2 is coupled to the CP2 via the CM4. In some embodiments, the MM2 is coupled to the CP2 via the CM2.

In some embodiments, the ACC further comprises a third monomer comprising a Sushi domain comprising of the sequence of SEQ ID NO: 520. In some embodiments, the ACC further a fourth monomer comprising a Sushi domain comprising of the sequence of SEQ ID NO: 520.

In some embodiments, the third monomer further comprises a tag (e.g., a peptide tag such as a His tag, myc tag, etc.). In some embodiments, the fourth monomer further comprises a tag (e.g., a peptide tag such as a His tag, myc tag, etc.).

In some embodiments, the ACC comprises a first monomer construct and a second monomer construct, wherein
the first monomer construct comprises a first cytokine polypeptide (CP1), a first dimerization domain (DD1), and a first masking moiety (MM1),
the second monomer construct comprises a second cytokine polypeptide (CP2), a first cleavable moiety (CM1), a second dimerization domain (DD2) coupled to the CP2 via the CM1, and a second masking moiety (MM2),
the MM1 and/or the MM2 is the MM described herein, and
the DD1 and DD2 bind each other thereby forming a dimer of the first and second monomer constructs.

In some embodiments the first monomer construct further comprises a second cleavable moiety (CM2) and the MM1 is coupled to the CP1 via the CM2. In some embodiments the MM2 is coupled to the CP2 via the CM1. In some embodiments, the second monomer construct further comprises a third cleavable moiety (CM3), wherein the MM2 is coupled to the CP2 via the CM3.

In some embodiments, the ACC comprises a first monomer construct and a second monomer construct, wherein
the first monomer construct comprises a first cytokine polypeptide (CP1), a first dimerization domain (DD1), and a first masking moiety (MM1),
the second monomer construct comprises a second cytokine polypeptide (CP2), a second dimerization domain (DD2), and a second masking moiety (MM2),
the CP1 and/or the CP2 comprises an amino acid sequence that functions as a substrate for a protease, and the DD1 and/or DD2 is coupled to the CP1 and/or CP2 via the amino acid sequence,
the MM1 and/or the MM2 is the MM described herein, and
the DD1 and DD2 bind each other thereby forming a dimer of the first and second monomer constructs.

In some embodiments, the CP1 comprises an amino acid sequence that functions as a substrate for a protease, and the MM1 is coupled to the CP1 via the amino acid sequence. In some embodiments, the first monomer construct further comprises a first cleavable moiety (CM1) and the MM1 is coupled to the CP1 via the CM1. In some embodiments, the CP2 comprises an amino acid sequence that functions as a substrate for a protease, and the MM2 is coupled to the CP2 via the amino acid sequence. In some embodiments, the second monomer construct further comprises a second cleavable moiety (CM2) and the MM2 is coupled to the CP2 via the CM2.

In some embodiments, the ACC comprises a first monomer construct and a second monomer construct, wherein
the first monomer construct comprises a cytokine polypeptide (CP), a first dimerization domain (DD1), a first cleavable moiety (CM1), a second cleavable moiety (CM2), and the MM described herein, wherein the MM is coupled to the CP via the CM1, and the DD1 is coupled to the CP via the CM2,
the second monomer construct comprises an agonist of the CP, a third cleavable moiety (CM3), a second dimerization domain (DD2) coupled to the agonist via the CM3,
the DD1 and DD2 bind each other thereby forming a dimer of the first and second monomer constructs.

In some embodiments, the CP is IL-15, and the agonist is a Sushi domain comprising the sequence of SEQ ID NO: 520. In some embodiments, the ACC comprises a linker between the Sushi domain and the CM3. In some aspects, the linker may include 1-10 amino acids, e.g., 1, 2, 3, 4, 5, 6, 7, 8, 9, or 10 amino acids. In some aspects, the linker may include 1-3 amino acids, e.g., 1, 2, or 3 amino acids, or in some aspects consist of 2 amino acids.

In an ACC that is a dimer complex, the first and second monomer constructs may further comprise additional elements, such as, for example, one or more linkers, and the like. The additional elements are described below in more detail. The organization of the CP, CM, MM, and DD components in each of the first and second monomer constructs may be arranged in the same order in each monomer construct. The CP1, CM1, MM1, and DD1 components may be the same or different as compared to the corresponding CP2, CM2, MM2, and DD2, in terms of, for example, molecular weight, size, amino acid sequence of the CP and CM components (and the DD components in embodiments where the DD components are polypeptides), and the like. Thus, the resulting dimer may have symmetrical or asymmetrical monomer construct components.

In some embodiments, the first monomer construct comprises, from N- to C-terminus of the CP and CM components, the CP1, the CM1, and, linked directly or indirectly (via a linker) to the C-terminus of the CM1, the DD1. In other embodiments, the first monomer construct comprises from C- to N-terminus of the CP and CM components, the CP1, the CM1, and, linked directly or indirectly (via a linker) to the N-terminus of the CM1, the DD1. In some embodiments, the second monomer construct comprises, from N- to C-terminal terminus of the CP and CM components, the CP2, the CM2, and, linked directly or indirectly (via a linker) to the C-terminus of the CM2, the DD2. In other embodiments, the second monomer construct comprises, from C- to N-terminus of the CP and CM components, the CP2, the CM2, and, linked directly or indirectly (via a linker) to the N-terminus of the CM2, the DD2. In some embodiments, the first monomer comprising the first mature cytokine polypeptide (CP1) and/or the second monomer comprising the second mature cytokine polypeptide (CP2) comprises one or more MMs. In some embodiments, the ACC further comprises a CM between the MM and the CP.

In some embodiments, the activatable cytokine constructs (ACC) that include a first monomer construct and a second monomer construct, wherein: (a) the first monomer construct comprises a first masking moiety (MM1), a first mature cytokine polypeptide (CP1), a first and a third cleavable moieties (CM1 and CM3), and a first dimerization domain (DD1), wherein the CM1 is positioned between the CP1 and the DD1, and the CM3 is positioned between the MM1 and the CP1; and (b) the second monomer construct comprises a second mature cytokine polypeptide (CP2), a second cleavable moiety (CM2), and a second dimerization domain (DD2), wherein the CM2 is positioned between the CP2 and the DD2; wherein the DD1 and the DD2 bind each other thereby forming a dimer of the first monomer construct and the second monomer construct; and wherein the ACC is characterized by having a reduced level of at least one CP1 and/or CP2 activity as compared to a control level of the at least one CP1 and/or CP2 activity.

In some embodiments, the second monomer construct further comprises a second masking moiety (MM2) and a fourth cleavable moiety (CM4), wherein the CM4 is positioned between the MM2 and the CP2. In some embodiments, the first monomer construct comprises a first polypeptide that comprises the MM1, the CM3, the CP1, the CM1, and the DD1. In some embodiments, the second monomer construct comprises a second polypeptide that comprises the CP2, the CM2, and the DD2. In some embodiments, the second monomer construct comprises a second polypeptide that comprises the MM2, the CM4, the CP2, the CM2, and the DD2.

The ACC structure was discovered to be highly effective at reducing activity of the mature cytokine polypeptide components in a way that does not lead to substantially impaired cytokine activity after activation. The CP's activity in the ACC may be reduced by both the structure of the ACC (e.g., the dimer structure) and the masking moiet(ies) in the ACC. In some embodiments, the activation condition for the ACCs described herein is exposure to one or more proteases that can dissociate the CP from both the DD and the MM. For example, the one or more proteases may cleave the CM between the CP and the MM and the CM between the CP and the DD. As demonstrated in the Examples, activation of the ACC resulted in substantial recovery of cytokine activity. The results suggest that conformation of the cytokine components was not irreversibly altered within the context of the ACC.

In some embodiments, when a cytokine polypeptide is coupled to a MM and in the presence of a natural binding partner of the cytokine polypeptide (e.g., its receptor), there is no binding or substantially no binding of the cytokine polypeptide to the binding partner, or no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding of the cytokine polypeptide to its binding partner, as compared to the binding of the cytokine polypeptide not coupled to a MM, for at least 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, 12 months or greater when measured in a mask efficiency assay. For example, the mask efficiency assay may involve measurement of the affinity of an ACC binding to a cell surface displaying a candidate masking moiety by, for example, FACS. Another non-limiting exemplary assay includes assessing the ability of a masking moiety to inhibit ACC binding to its binding partner at therapeutically relevant concentrations and times. For this second method, an immunoabsorbant assay to measure the time-dependent binding of proprotein binding to its binding partner has been developed as described in US20200308243, incorporated herein by reference. In an embodiment, the mask efficiency assay may involve measuring a level of secreted alkaline phosphatase (SEAP) production in IL-2/IL15-responsive HEK293 cells.

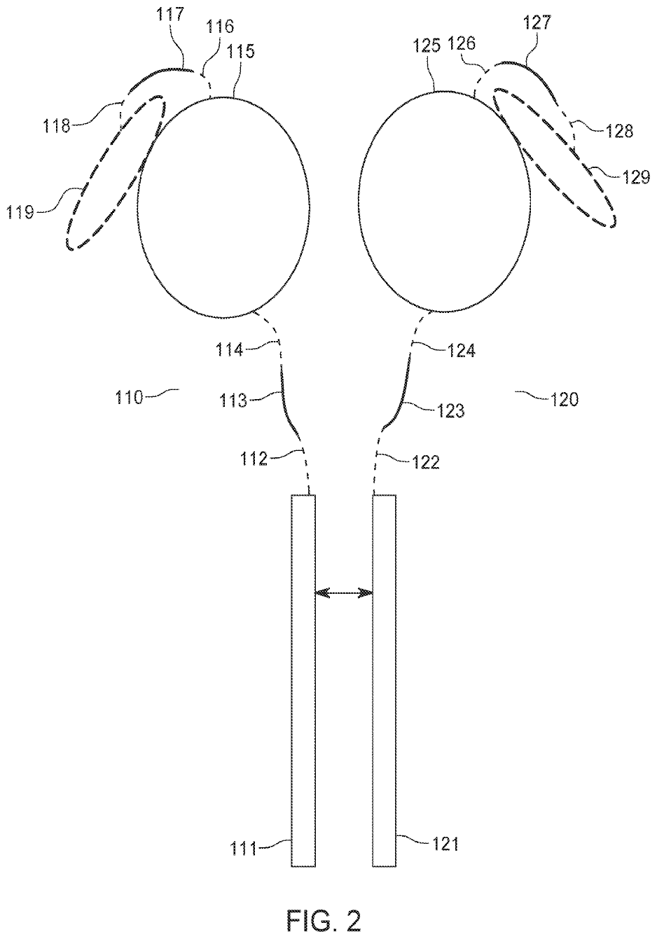

In certain embodiments, the first and second monomeric constructs are oriented such that the components in each member of the dimer are organized in the same order from N-terminus to C-terminus of the CP and CM components. FIG. 2 is a schematic of an illustrative activatable cytokine construct comprising, from N-terminus to C-terminus: (1) a first monomer construct 110 having optionally a MM1 119, optionally a CM3 117, a CP1 115, a CM1 113, and a DD1 111, and; (2) a second monomer construct 120 having optionally a MM2 129, optionally a CM4 127, a CP2 125, a CM2 123, and a DD2 121; and (3) one or more covalent or non-covalent bonds (←→) bonding the first monomer construct 110 to the second monomer construct 120. The ACC may further comprise one or more of the optional linkers 112, 114, 116, 118, 122, 124, 126, and 128 between the components. In one example, DD1 111 and DD2 121 are the same. In another example, DD1 111 and DD2 121 are different. In some examples, DD1 111 and DD2 121 are different polypeptides that bind to each other.

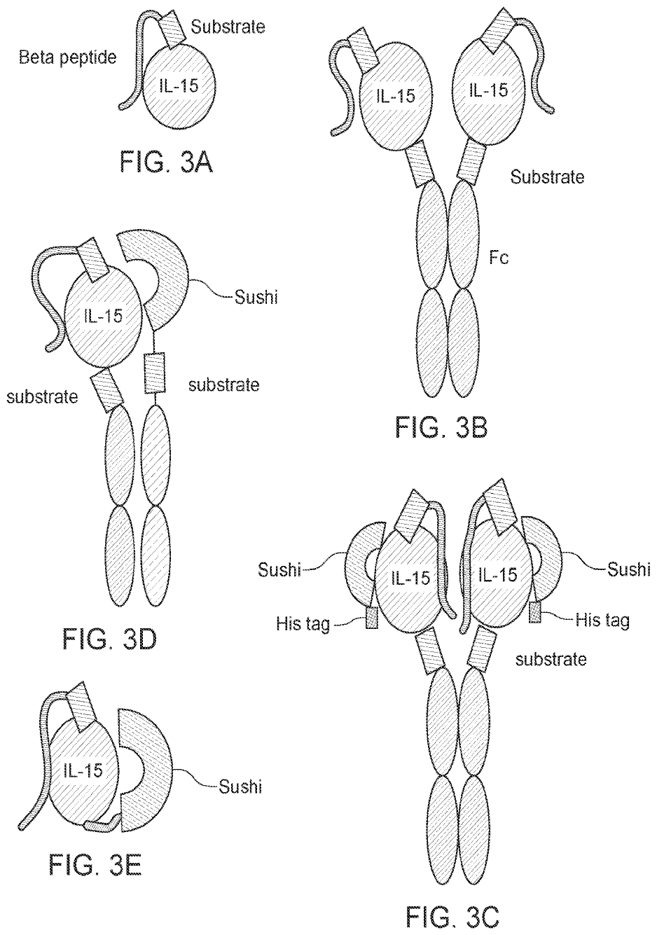

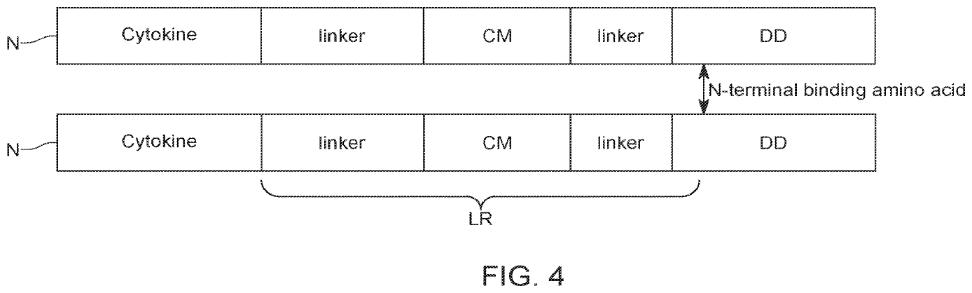

In alternative aspects, one of the two moieties depicted as CP1 115 and CP2 125 is a mutated cytokine polypeptide that lacks cytokine activity. In alternative aspects, one of the two moieties depicted as CP1 115 and CP2 125 is a polypeptide sequence that lacks cytokine activity, e.g., a signal moiety and/or a stub sequence. In alternative aspects, a first one of the two moieties depicted as CP1 115 and CP2 125 is a polypeptide sequence that binds with high affinity to a second one of the two moieties depicted as CP1 115 and CP2 125 and reduces the cytokine activity of the second moiety as compared to the control level of the second moiety. Additional example embodiments of ACCs are shown in FIGS. 3A-3E. Any depictions of substrates attached to masking moieties in these figures are optional and exemplary aspects of the invention and the CM or the CM-MM features are optional and non-limiting. Examples of constructs of FIG. 3C include ProC2982 (SEQ ID NO: 525 complexed with SEQ ID NO: 526 and dimerized with a second identical monomer construct complex) and examples of constructs of FIG. 3D include ProC3571 (SEQ ID NO: 528 dimerized with SEQ ID NO: 527 using a knob-hole Fc dimer). In some aspects, the ACC may include a cytokine polypeptide, e.g., IL-15, or a biologically active fragment thereof, a CM, and a MM. In some aspects, the ACC may include a first monomer construct and a second monomer construct, each monomer construct including a cytokine polypeptide, e.g., IL-15, or a biologically active fragment thereof, a CM, MM, and a DD, wherein the first and second monomer constructs are dimerized via the DDs. In some aspects, the ACC may include a first monomer construct including a cytokine polypeptide, e.g., IL-15, or a biologically active fragment thereof, a CM, a MM, and a DD, and a second monomer construct including a sushi domain or fragment thereof, a CM, and DD, wherein the first and second monomer constructs are dimerized via the DDs. In some aspects, the ACC may include a first monomer construct including a cytokine polypeptide, e.g., IL-15, or a biologically active fragment thereof linked to a DD1 via a CM1 and a second monomer construct including an MM linked to a DD2, wherein the first and second monomer constructs are dimerized via the DD1 and the DD2. In some aspects, the MM is linked to the DD2 via a CM2 on the second monomer construct. In some aspects, the cytokine polypeptide is linked to the CM1 via a sushi domain on the first monomer construct. In some aspects, the ACC may include a first monomer construct including a cytokine polypeptide, e.g., IL-15, or a biologically active fragment thereof linked to a DD1 and a second monomer construct including an MM linked to a DD2 via a CM1, wherein the first and second monomer constructs are dimerized via the DDs. In some aspects, the first monomer construct includes a sushi domain linked to the cytokine polypeptide via a CM2. In some aspects, the ACC may include a cytokine polypeptide, e.g., IL-15, or a biologically active fragment thereof, a CM, a MM, and the IL-15 is linked to a sushi domain or fragment thereof. In some aspects, the ACC may include a first monomer construct and a second monomer construct, each monomer construct including a cytokine polypeptide, e.g., IL-15, or a biologically active fragment thereof, a CM, MM, and a DD, wherein the first and second monomer constructs are dimerized via the DDs, and wherein each IL-15 is bound to a sushi domain or fragment thereof.

The activation condition for the ACCs described herein is exposure to a protease that can cleave at least one of the cleavable moieties (CMs) in the ACC. As demonstrated in the Examples, activation of the ACC resulted in substantial recovery of cytokine activity. The results suggest that conformation of the cytokine components was not irreversibly altered within the context of the ACC.

The mature cytokine polypeptides, CP1 and CP2 is the same or different. In certain specific embodiments, CP1 and CP2 are the same. In other embodiments, CP1 and CP2 are different. The ACC may comprise additional amino acid residues at either or both N- and/or C-terminal ends of the CP1 and/or CP2.

Dimerization domains (DDs) Each monomer construct of an ACC that is a dimer complex may employ any of a variety of dimerization domains (DDs). Suitable DDs include both polymeric (e.g., a synthetic polymer, a polypeptide, a polynucleotide, and the like) and small molecule (non-polymeric moieties having a molecular weight of less than about 1 kilodalton, and sometimes less than about 800 Daltons) types of moieties. The pair of DDs is any pair of moieties that are known in the art to bind to each other.

For example, in some embodiments, the DD1 and the DD2 are members of a pair selected from the group of: a sushi domain from an alpha chain of human IL-15 receptor (IL15Rα) and a soluble IL-15; barnase and barnstar; a PKA and an AKAP; adapter/docking tag molecules based on mutated RNase I fragments; a pair of antigen-binding domains (e.g., a pair of single domain antibodies); soluble N-ethyl-maleimide sensitive factor attachment protein receptors (SNARE) modules based on interactions of the proteins syntaxin, synaptotagmin, synaptobrevin, and SNAP25; a single domain antibody (sdAb) and corresponding epitope; an antigen-binding domain (e.g., a single chain antibody such as a single chain variable fragment (scFv), a single domain antibody, and the like) and a corresponding epitope; coiled coil polypeptide structures (e.g., Fos-Jun coiled coil structures, acid/base coiled-coil helices, Glu-Lys coiled coil helices, leucine zipper structures), small molecule binding pairs such as biotin and avidin or streptavidin, amine/aldehyde, lectin/carbohydrate; a pair of polymers that can bind each other, such as, for example, a pair of sulfur- or thiol-containing polymers (e.g., a pair of Fc domains, a pair of thiolized-human serum albumin polypeptides, and the like); and the like.

In some embodiments, the DD1 and DD2 are non-polypeptide polymers. The non-polypeptide polymers may covalently bound to each other. In some examples, the non-polypeptide polymers are a sulfur-containing polymer, e.g., sulfur-containing polyethylene glycol. In such cases, the DD1 and DD2 is covalently bound to each other via one or more disulfide bonds.

When the pair of DD1 and DD2 are members of a pair of epitope and antigen-binding domain, the epitope may be a naturally or non-naturally occurring epitope. Exemplary non-naturally occurring epitopes include, for example, a non-naturally occurring peptide, such as, for example, a poly-His peptide (e.g., a His tag, and the like).

In certain specific embodiments, the DD1 and the DD2 are a pair of Fc domains. As used herein, an “Fc domain” refers to a contiguous amino acid sequence of a single heavy chain of an immunoglobulin. A pair of Fc domains associate together to form an Fc region of an immunoglobulin.

In some embodiments, the pair of Fc domains is a pair of human Fc domains (e.g., a pair of wild type human Fc domains). In some embodiments, the human Fc domains are human IgG1 Fc domains (e.g., wildtype human IgG1 Fc domains), human IgG2 Fc domains (e.g., wildtype human IgG2 Fc domains), human IgG3 Fc domains (e.g., wildtype human IgG3 Fc domains), or human IgG4 Fc domains (e.g., wildtype human IgG4 Fc domains). In some embodiments, the human Fc domains comprise a sequence that is at least 80% identical (e.g., at least 82%, at least 84%, at least 85%, at least 86%, at least 88%, at least 90%, at least 92%, at least 94%, at least 95%, at least 96%, at least 97%, at least 98%, at least 99%, or 100% identical) to SEQ ID NO: 3.

In some embodiments, the pair of Fc domains comprises a knob mutant and a hole mutant of a Fc domain. The knob and hole mutants may interact with each other to facilitate the dimerization. In some embodiments, the knob and hole mutants may comprise one or more amino acid modifications within the interface between two Fc domains (e.g., in the CH3 domain). In one example, the modifications comprise amino acid substitution T366W and optionally the amino acid substitution S354C in one of the antibody heavy chains, and the amino acid substitutions T366S, L368A, Y407V and optionally Y349C in the other one of the antibody heavy chains (numbering according to EU index of Kabat numbering system). Examples of the knob and hole mutants include Fc mutants of SEQ ID NOs: 315 and 316, as well as those described in U.S. Pat. Nos. 5,731,168; 7,695,936; and 10,683,368, which are incorporated herein by reference in their entireties. In some embodiments, the dimerization domains comprise a sequence that is at least 80% identical (e.g., at least 82%, at least 84%, at least 85%, at least 86%, at least 88%, at least 90%, at least 92%, at least 94%, at least 95%, at least 96%, at least 97%, at least 98%, at least 99%, or 100% identical) to SEQ ID NOs: 315 and 316, respectively. In some embodiments, the human Fc domains comprise the mutation N297Q, N297A, or N297G; in some embodiments the human Fc domains comprise a mutation at position 234 and/or 235, for example L235E, or L234A and L235A (in IgG1), or F234A and L235A (in IgG4); in some embodiments the human Fc domains are IgG2 Fc domains that comprise the mutations V234A, G237A, P238S, H268Q/A, V309L, A330S, or P331S, or a combination thereof (all according to EU numbering). Additional examples of engineered human Fc domains are known to those skilled in the art. Examples of Ig heavy chain constant region amino acids in which mutations in at least one amino acid leads to reduced Fc function include, but are not limited to, mutations in amino acid 228, 233, 234, 235, 236, 237, 239, 252, 254, 256, 265, 270, 297, 318, 320, 322, 327, 329, 330, and 331 of the heavy constant region (according to EU numbering). Examples of combinations of mutated amino acids are also known in the art, such as, but not limited to a combination of mutations in amino acids 234, 235, and 331, such as L234F, L235E, and P331S or a combination of amino acids 318, 320, and 322, such as E318A, K320A, and K322A.

Further examples of engineered Fc domains include F243L/R292P/Y300L/V305I/P396 IgG1; S239D/I332E IgG1; S239D/I332E/A330L IgG1; S298A/E333A/K334A; in one heavy chain, L234Y/L235Q/G236W/S239M/H268D/D270E/S298A IgG1, and in the opposing heavy chain, D270E/K326D, A330M/K334E IgG; G236A/S239D/I332E IgG1; K326W/E333S IgG1; S267E/H268F/S324T IgG1; E345R/E430G/S440Y IgG1; N297A or N297Q or N297G IgG1; L235E IgG1; L234A/L235A IgG1; F234A/L235A IgG4; H268Q/V309L/A330S/P331S IgG2; V234A/G237A/P238S/H268A/V309L/A330S/P331S IgG2; M252Y/S254T/T256E IgG1; M428L/N434S IgG1; S267E/L328F IgG1; N325S/L328F IgG1, and the like. In some embodiments, the engineered Fc domain comprises one or more substitutions selected from the group consisting of N297A IgG1, N297Q IgG1, and S228P IgG4.

In some aspects, the dimerization domain is an IgG Fc region wherein the upper hinge residues have been deleted. For example, the Fc is a variant wherein N-terminal sequences EPKSCDKTHT (SEQ ID NO: 387), ERK, ELKTPLGDTTHT (SEQ ID NO: 388), or ESKYGPP (SEQ ID NO: 389) have been deleted.

In some aspects, the DD or the DD1 and/or DD2, can further include a serum half-life extending moiety (e.g., polypeptides that bind serum proteins, such as immunoglobulin (e.g., IgG) or serum albumin (e.g., human serum albumin (HSA)). Examples of half-life extending moieties include hexa-hat GST (glutathione S-transferase) glutathione affinity, Calmodulin-binding peptide (CBP), Strep-tag, Cellulose Binding Domain, Maltose Binding Protein, S-Peptide Tag, Chitin Binding Tag, Immuno-reactive Epitopes, Epitope Tags, E2Tag, HA Epitope Tag, Myc Epitope, FLAG Epitope, AU1 and AU5 Epitopes, Glu-Glu Epitope, KT3 Epitope, IRS Epitope, Btag Epitope, Protein Kinase-C Epitope, and VSV Epitope.

In some embodiments, DD1 and/or DD2 each include a total of about 5 amino acids to about 250 amino acids, about 5 amino acids to about 200 amino acids, about 5 amino acids to about 180 amino acids, about 5 amino acids to about 160 amino acids, about 5 amino acids to about 140 amino acids, about 5 amino acids to about 120 amino acids, about 5 amino acids to about 100 amino acids, about 5 amino acids to about 80 amino acids, about 5 amino acids to about 60 amino acids, about 5 amino acids to about 40 amino acids, about 5 amino acids to about 20 amino acids, about 5 amino acids to about 10 amino acids, about 10 amino acids to about 250 amino acids, about 10 amino acids to about 200 amino acids, about 10 amino acids to about 180 amino acids, about 10 amino acids to about 160 amino acids, about 10 amino acids to about 140 amino acids, about 10 amino acids to about 120 amino acids, about 10 amino acids to about 100 amino acids, about 10 amino acids to about 80 amino acids, about 10 amino acids to about 60 amino acids, about 10 amino acids to about 40 amino acids, about 10 amino acids to about 20 amino acids, about 20 amino acids to about 250 amino acids, about 20 amino acids to about 200 amino acids, about 20 amino acids to about 180 amino acids, about 20 amino acids to about 160 amino acids, about 20 amino acids to about 140 amino acids, about 20 amino acids to about 120 amino acids, about 20 amino acids to about 100 amino acids, about 20 amino acids to about 80 amino acids, about 20 amino acids to about 60 amino acids, about 20 amino acids to about 40 amino acids, about 40 amino acids to about 250 amino acids, about 40 amino acids to about 200 amino acids, about 40 amino acids to about 180 amino acids, about 40 amino acids to about 160 amino acids, about 40 amino acids to about 140 amino acids, about 40 amino acids to about 120 amino acids, about 40 amino acids to about 100 amino acids, about 40 amino acids to about 80 amino acids, about 40 amino acids to about 60 amino acids, about 60 amino acids to about 250 amino acids, about 60 amino acids to about 200 amino acids, about 60 amino acids to about 180 amino acids, about 60 amino acids to about 160 amino acids, about 60 amino acids to about 140 amino acids, about 60 amino acids to about 120 amino acids, about 60 amino acids to about 100 amino acids, about 60 amino acids to about 80 amino acids, about 80 amino acids to about 250 amino acids, about 80 amino acids to about 200 amino acids, about 80 amino acids to about 180 amino acids, about 80 amino acids to about 160 amino acids, about 80 amino acids to about 140 amino acids, about 80 amino acids to about 120 amino acids, about 80 amino acids to about 100 amino acids, about 100 amino acids to about 250 amino acids, about 100 amino acids to about 200 amino acids, about 100 amino acids to about 180 amino acids, about 100 amino acids to about 160 amino acids, about 100 amino acids to about 140 amino acids, about 100 amino acids to about 120 amino acids, about 120 amino acids to about 250 amino acids, about 120 amino acids to about 200 amino acids, about 120 amino acids to about 180 amino acids, about 120 amino acids to about 160 amino acids, about 120 amino acids to about 140 amino acids, about 140 amino acids to about 250 amino acids, about 140 amino acids to about 200 amino acids, about 140 amino acids to about 180 amino acids, about 140 amino acids to about 160 amino acids, about 160 amino acids to about 250 amino acids, about 160 amino acids to about 200 amino acids, about 160 amino acids to about 180 amino acids, about 180 amino acids to about 250 amino acids, about 180 amino acids to about 200 amino acids, about 200 amino acids to about 250 amino acids, about 210 to about 220 amino acids, about 215 to about 225 amino acids, about 215 to about 220 amino acids, about 217 to about 200 amino acids, or about 218 to about 200 amino acids. In some embodiments, DD1 and DD2 are each an Fc domain that comprises a portion of the hinge region that includes two cysteine residues, a CH2 domain, and a CH3 domain. In some embodiments, DD1 and DD2 are each an Fc domain whose N-terminus is the first cysteine residue in the hinge region reading in the N- to C-direction (e.g., Cysteine 226 of human IgG1 or IgG4, using EU numbering).

In some embodiments, the first monomer and/or the second monomer can each include a total of about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 250 amino acids, about 150 amino acids to about 200 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 250 amino acids, about 250 amino acids to about 800 amino acids, about 250 amino acids to about 750 amino acids, about 250 amino acids to about 700 amino acids, about 250 amino acids to about 650 amino acids, about 250 amino acids to about 600 amino acids, about 250 amino acids to about 550 amino acids, about 250 amino acids to about 500 amino acids, about 250 amino acids to about 450 amino acids, about 250 amino acids to about 400 amino acids, about 250 amino acids to about 350 amino acids, about 250 amino acids to about 300 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 800 amino acids, about 350 amino acids to about 750 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 800 amino acids, about 450 amino acids to about 750 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 800 amino acids, about 600 amino acids to about 750 amino acids, about 600 amino acids to about 700 amino acids, about 600 amino acids to about 650 amino acids, about 650 amino acids to about 800 amino acids, about 650 amino acids to about 750 amino acids, about 650 amino acids to about 700 amino acids, about 700 amino acids to about 800 amino acids, about 700 amino acids to about 750 amino acids, or about 750 amino acids to about 800 amino acids.

### Cleavable Moieties (CMs)

In some embodiments, the ACC comprises one or more CMs. A CM is positioned between two components in an ACC, e.g., between a cytokine polypeptide and a MM, between a cytokine polypeptide and a DD, and/or between a cytokine polypeptide and another component in the ACC. In some embodiments, a MM is coupled with a cytokine polypeptide via a CM, i.e., the CM is positioned between the interleukin and the MM.

In some embodiments, a CM is positioned between a MM and a cytokine polypeptide, either directly or indirectly (e.g., via a linker). In some embodiments, a CM is positioned between the cytokine polypeptide and a DD, either directly or indirectly (e.g., via a linker).

In some embodiments, the CMs herein may comprise substrates for proteases that have been reported in a cancer, or in a number of cancers. See, e.g., La Roca et al., British J. Cancer 90(7):1414-1421, 2004. Substrates suitable for use in the CM component employed herein include those which are more prevalently found in cancerous cells and tissue. Thus, in certain embodiments, a CM comprises a substrate for a protease that is more prevalently found in diseased tissue associated with a cancer. In some embodiments, the cancer is selected from the group of: gastric cancer, breast cancer, osteosarcoma, and esophageal cancer. In some embodiments, the cancer is breast cancer. In some embodiments, the cancer is a HER2-positive cancer. In some embodiments, the cancer is Kaposi sarcoma, hairy cell leukemia, chronic myeloid leukemia (CML), follicular lymphoma, renal cell cancer (RCC), melanoma, neuroblastoma, basal cell carcinoma, cutaneous T-cell lymphoma, nasopharyngeal adenocarcinoma, breast cancer, ovarian cancer, bladder cancer, BCG-resistant non-muscle invasive bladder cancer (NMIBC), endometrial cancer, pancreatic cancer, non-small cell lung cancer (NSCLC), colorectal cancer, esophageal cancer, gallbladder cancer, glioma, head and neck carcinoma, uterine cancer, cervical cancer, or testicular cancer, and the like. In some of the above-described embodiments, the CM components comprise substrates for protease(s) that is/are more prevalent in tumor tissue. For example, the protease(s) may be produced by a tumor in a subject.

Suitable CMs for use in the ACCs herein include any of the protease substrates that are known the art. In some examples, the CM may comprise a substrate of a serine protease (e.g., u-type plasminogen activator (uPA, also referred to as urokinase), a matriptase (also referred to herein as MT-SP1 or MTSP1). In some examples, the CM may comprise a substrate of a matrix metalloprotease (MMP). In some examples, the CM may comprise a substrate of cysteine protease (CP) (e.g., legumain).

In some embodiments, the CM may comprise a substrate for a disintegrin and a metalloproteinase (ADAM) or a disintegrin and a metalloproteinase with a thrombospondin motifs (ADAMTS) (e.g., ADAM8, ADAM9, ADAM10, ADAM12, ADAM15, ADAM17/TACE, ADEMDEC1, ADAMTS1, ADAMTS4, ADAMTS5), an aspartate protease (e.g. BACE, Renin), an aspartic cathepsin (e.g., Cathepsin D, Cathepsin E), Caspase (e.g., Caspase 1, Caspase 2, Caspase 3, Caspase 4, Caspase 5, Caspase 6, Caspase 7, Caspase 8, Caspase 9, Caspase 10, Caspase 14), cysteine cathepsin (e.g., Cathepsin A, Cathepsin B, Cathepsin C, Cathepsin G, Cathepsin K, Cathepsin L, Cathepsin S, Cathepsin V/L2, Cathepsin X/Z/P), a cysteine proteinase (e.g., Cruzipain, Legumain, Otubain-2), a Chymase, DESC1, DPP-4, FAP, an Elastase, FVIIa, FiXA, FXa, FXIa, FXIIa, Granzyme B, Guanidinobenzoatase, Hepsin, HtrA1, Human a Neutrophil Elastase, a KLK (e.g., KLK4, KLK5, KLK6, KLK7, KLK8, KLK10, KLK11, KLK13, KLK14), a metallo proteinase (e.g., Meprin, Neprilysin, PSMA, BMP-1), Lactoferrin, Marapsin, Matriptase-2, MT-SP1/Matriptase, NS3/4A, PACE4, Plasmin, PSA, an MMP (e.g., MMP1, MMP2, MMP3, MMP7, MMP8, MMP9, MMP10, MMP11, MMP12, MMP13, MMP14, MMPP15, MMPP16, MMP17, MMP19, MMP20, MMP23, MMP24, MMP26, MMP27), TMPRSS2, TMPRSS3, TMPRSS4, tPA, Thrombin, Tryptase, and uPA.

In some embodiments, the protease substrate in the CM may comprise a polypeptide sequence that is not substantially identical (e.g., no more than 90%, 80%, 70%, 60%, or 50% identical) to any polypeptide sequence that is naturally cleaved by the same protease.

In some embodiments, CM comprises or consists of a sequence of LSGRSDNH (SEQ ID NO: 552) or PLGLAG (SEQ ID NO: 615). In some embodiments, the CM comprises or consists of a sequence of encompassed by the consensus of sequence of any one of SEQ ID NOs: 317-327, 329-335, 340-347, 352-363, 371-378, 394-401, 410-419, 425-433, 436-449, 453-456, 458-469, 473, 475-482, 485-495 disclosed in WO2015048329, which is incorporated by reference herein in its entirety, and SEQ ID NOs: 1-162, 268-306 disclosed in WO2015116933, which is incorporated by reference herein in its entirety.

In some embodiments, the CM comprises or consists of a sequence of any one of SEQ ID NOs: 14-52, 126-154. 159, 315-316, 328, 336-339, 348-351, 364-370, 379-393, 402-409, 420-424, 434-435, 450-452, 457, 470-472, 474, 483, 484 disclosed in WO2015048329, SEQ ID NOs: 163-267, 307-384, 402-445, 665-683 disclosed in WO2015116933, SEQ ID NOs: 20-21, 411, 480-482, 351-369, 18, 71, 370-380, 412-415, 468, 547-554, 319-346 disclosed in WO2016118629, which is incorporated by reference herein in its entirety, and SEQ ID NOs: 1-16, 50-56, 60-63, 20, 70-76, 78-115, 120-128, 130-132, 135-140, 141, 152, 21-23, 17-19, 25-43 disclosed in WO2020118109, which is incorporated by reference herein in its entirety. In some examples, the CM of a cysteine protease comprises or consists of the sequence of AAN, SAN, or GPTN (SEQ ID NO: 152). Examples of CMs also include those described in WO 2010/081173, WO2021207669, WO2021207657, WO2021142029, WO2021061867, WO2020252349, WO2020252358, WO2020236679, WO2020176672, WO2020118109, WO2020092881, WO2020086665, WO2019213444, WO2019183218, WO2019173771, WO2019165143, WO2019075405, WO2019046652, WO2019018828, WO2019014586, WO2018222949, WO2018165619, WO2018085555, WO2017011580, WO2016179335, WO2016179285, WO2016179257, WO2016149201, WO2016014974, which are incorporated herein by reference in their entireties.

In some embodiments, the CM comprises or consists of a sequence or encompassed by the consensus of sequence of any one of the sequences in the Table 1 with CM sequences below.

TABLE1
CM Sequences

| | SEQ |
|---|---|
| Sequences | ID NO |
| AANALAHGLF | 5 |
| AANL | 6 |
| AANLGSGGSS | 7 |
| AAPRS | 8 |
| AAPRSF | 9 |
| AARGPAIH | 10 |
| AAYHLVSQ | 11 |
| AFPDMRSVRS | 12 |
| AFQALRM | 13 |
| AFRHLR | 14 |
| AGLGISST | 15 |
| AGLGVVER | 16 |
| AGPR | 17 |
| AHGL | 18 |
| AHGLF | 19 |
| AHQALRM | 20 |
| AIPRVRLFDV | 21 |
| ALAHG | 22 |
| ALAHGL | 23 |
| ALAHGLF | 24 |
| ALAHGLFAPRSF | 25 |
| ALAHGLFSGRSAN | 26 |
| ALAHGLPTFVHL | 27 |
| ALGLLRLP | 28 |
| ALPSVKMVSE | 29 |
| ALRAP | 30 |
| ANQALRM | 31 |
| ANQALRMA | 32 |
| APPLVKSMVV | 33 |
| APPSFKLVNA | 34 |
| APRS | 35 |
| APRSALAHGLF | 36 |
| APRSF | 37 |
| AQFVLTEG | 38 |
| AQNLLGMV | 39 |
| ARGP | 40 |
| ARGPS | 41 |
| ARGPSF | 42 |
| ARGPSFK | 43 |
| ASGLLRFP | 44 |
| ASPTMKTVGL | 45 |
| AVGLLAPP | 46 |
| AVGLLAPPGGLSGRSANI | 47 |
| AVGLLAPPGGLSGRSANP | 48 |
| AVGLLAPPGGLSGRSDDH | 49 |
| AVGLLAPPGGLSGRSDIH | 50 |
| AVGLLAPPGGLSGRSDNH | 51 |
| AVGLLAPPGGLSGRSDNI | 52 |
| AVGLLAPPGGLSGRSDNP | 53 |
| AVGLLAPPGGLSGRSDQH | 54 |
| AVGLLAPPGGLSGRSDTH | 55 |
| AVGLLAPPGGLSGRSDYH | 56 |
| AVGLLAPPGGLSGRSNI | 57 |
| AVGLLAPPGGLSGRSNIG | 58 |
| AVGLLAPPGGLSGRSNIGS | 59 |
| AVGLLAPPGGTSTSGRSANPRG | 60 |
| AVGLLAPPSGRSANPRG | 61 |
| AVGLLAPPTSGRSANPRG | 62 |
| AVPKVRVVPE | 63 |
| CGPPLGR | 64 |
| CSPPLGR | 65 |
| DEVDGSGGSS | 66 |
| DEXXXC(A/S) | 67 |
| DISHWRRS | 68 |
| DLAHPLL | 69 |
| DLPLVKSLPS | 70 |
| DLXXT(A/S) | 71 |
| DRLSGRSANHKK | 72 |
| DRLSGRSDNHKK | 73 |
| DRPEMKSLSG | 74 |
| DRPKVKTMDF | 75 |
| DVAQFVLT | 76 |
| DVPPMKTLRP | 77 |
| DWLYWMGI | 78 |
| DWLYWMSI | 79 |
| DWLYWPGI | 80 |
| DWLYWPSI | 81 |
| EAPKVKALPK | 82 |
| EHPRVKVVSE | 83 |
| EKPRMKLFQG | 84 |
| EPQALAMS | 85 |
| EQPEVKMVKG | 86 |
| ERPGVKSLVL | 87 |
| ESLPVVAV | 88 |
| ESPVMKSMAL | 89 |
| ESRRW | 90 |
| ESRRWM | 91 |
| ESRRWMP | 92 |
| ETPSVKTMGR | 93 |
| ETPSVKTMGRSS | 94 |
| FPRPLGITGL | 95 |
| FRLLDWQW | 96 |
| GCGPPLGR | 97 |
| GCSPPLGR | 98 |
| GFPHMKTFQH | 99 |
| GFPHMKTFQHSS | 100 |
| GGGPPLGR | 101 |
| GGPPLGR | 102 |
| GGQPSGMWGW | 103 |
| GGSIDGR | 104 |
| GGSPPLGR | 105 |
| GGWHTGRN | 106 |
| GIAGQ | 107 |
| GLGTPRGLFA | 108 |
| GLPTFV | 109 |
| GLPTFVH | 110 |
| GLPTFVHL | 111 |
| GLPTFVHLPRQV | 112 |
| GLSGRSDNHGSS | 113 |
| GPEGLRVG | 114 |
| GPLGIAGI | 115 |
| GPLNGRSDNHKA | 116 |
| GPLNGRSDNHKK | 117 |
| GPLNGRSDNHKR | 118 |
| GPLNGRSDNHQA | 131 |
| GPLNGRSDNHQK | 132 |
| GPLNGRSDNHQR | 133 |
| GPLNGRSDNHRA | 134 |
| GPLNGRSDNHRK | 135 |
| GPLNGRSDNHRR | 136 |
| GPLSGRSDNHKA | 137 |
| GPLSGRSDNHKK | 138 |
| GPLSGRSDNHKR | 139 |
| GPLSGRSDNHQA | 140 |
| GPLSGRSDNHQK | 141 |
| GPLSGRSDNHQR | 142 |
| GPLSGRSDNHRA | 143 |
| GPLSGRSDNHRK | 144 |
| GPLSGRSDNHRR | 145 |
| GPPLGR | 146 |
| GPQGIAGQ | 147 |
| GPQGLLGA | 148 |
| GPRSFG | 149 |
| GPRSFGL | 150 |
| GPSHLVLT | 151 |
| GPTN | 152 |
| GPTNALAHGLF | 153 |
| GRSML | 154 |
| GRSMLL | 155 |
| GRSMLLG | 156 |
| GRSMLLGG | 157 |
| GRSMLLGP | 158 |
| GRSMLLGS | 159 |
| GRSMLLP | 160 |
| GRSMLLPG | 161 |
| GRSMLLPP | 162 |
| GRSMLLPS | 163 |
| GRSMLLS | 164 |
| GRSMLLSG | 165 |
| GRSMLLSP | 166 |
| GRSMLLSS | 167 |
| GRSMLM | 168 |
| GRSMLMG | 169 |
| GRSMLMGG | 170 |
| GRSMLMGP | 171 |
| GRSMLMGS | 172 |
| GRSMLMP | 173 |
| GRSMLMPG | 174 |
| GRSMLMPP | 175 |
| GRSMLMPS | 176 |
| GRSMLMS | 177 |
| GRSMLMSG | 178 |
| GRSMLMSP | 179 |
| GRSMLMSS | 180 |
| GSGPPLGR | 181 |
| GSPPLGR | 182 |
| GSSPPLGR | 183 |
| GTGRGPSWVGSS | 184 |
| PTNL | 185 |
| PTNLGSGGSS | 186 |
| PVGYTSSL | 187 |
| PVPRLKLIKD | 188 |
| PVQPIGPQ | 189 |
| QALAMSAI | 190 |
| QFQALRM | 191 |
| QGPMFKSLWD | 192 |
| QGRAITFI | 193 |
| QHQALRM | 194 |
| QNQALRIA | 195 |
| QNQALRM | 196 |
| QNQALRMA | 197 |
| QNQALRMAGGSGGSLSGRSDN | 198 |
| H | |
| QNQALRMAGGSGGSLSGRSGN | 199 |
| H | |
| QSRRVP | 200 |
| QSRRVPL | 201 |
| QSRRVPV | 202 |
| QTRRVP | 203 |
| QTRRVPL | 204 |
| QTRRVPV | 205 |
| QYIVSRSA | 206 |
| RALRAP | 207 |
| REPFMKSLPW | 208 |
| RFPLKV | 209 |
| RFPSLKSFPL | 251 |
| RFPYGVW | 252 |
| RFYRNQFF | 253 |
| RGPA | 254 |
| RGPAFNPM | 255 |
| RGPATPIM | 256 |
| RGPKLYW | 257 |
| RHLAKL | 258 |
| RIGRSDNH | 259 |
| RKMPNITV | 260 |
| RKSSIIIRMRDVVL | 261 |
| RKTVQHWW | 262 |
| RLGRSDNN | 263 |
| RMHLRSLG | 264 |
| RPLARAGI | 265 |
| RPLARAGL | 266 |
| RPLNGRSDNHKA | 267 |
| RPLNGRSDNHKK | 268 |
| RPLNGRSDNHKR | 269 |
| RPLNGRSDNHQA | 270 |
| RPLNGRSDNHQK | 271 |
| RPLNGRSDNHQR | 272 |
| RPLNGRSDNHRA | 273 |
| RPLNGRSDNHRK | 274 |
| RPLNGRSDNHRR | 275 |
| RPLSGRSDNHKA | 276 |
| RPLSGRSDNHKK | 277 |
| RPLSGRSDNHKR | 278 |
| RPLSGRSDNHQA | 279 |
| RPLSGRSDNHQK | 280 |
| RPLSGRSDNHQR | 28 |
| RPLSGRSDNHRA | 282 |
| RPLSGRSDNHRK | 283 |
| RPLSGRSDNHRR | 284 |
| RPSPMWAY | 285 |
| RRHDGLRA | 286 |
| RRHDGLRS | 287 |
| RSLVFAPI | 288 |
| RSPSRLKC | 289 |
| VAGRSMRP | 290 |
| VAPQLKSLVP | 291 |
| VAQFVLTE | 292 |
| VHMPLGFLGP | 293 |
| VHMPLGFLGPGGLSGRSDNH | 294 |
| VHMPLGFLGPGGTSTSGRSANP | 295 |
| RG | |
| VLPELRSVFS | 296 |
| VLSKQMSF | 297 |
| VPAGRRS | 298 |
| VPAGRRSL | 299 |
| VPRQ | 300 |
| VPRQV | 301 |
| VSRSA | 302 |
| VVPEGRRS | 303 |
| WATPRPMR | 304 |
| WDHPISLL | 305 |
| QAR(A/V) | |
| YDPZVKVVLA | 307 |
| YGAGLGVV | 308 |
| YIVSRSA | 309 |
| YKKFVGSL | 310 |
| YVPRVKALEM | 311 |
| AQNLLGMY | 312 |
| VAQFVLT | 313 |
| HMMQYARH | 314 |
| HTGRSGAL | 432 |
| HVPRQ | 433 |
| HVPRQV | 434 |
| HVPRQVAPRSF | 435 |
| HVPRQVLSGRS | 436 |
| HVPRQVLSGRSAN | 437 |
| HWHLGPPT | 438 |
| IANLLSMV | 439 |
| IDGR | 440 |
| IEGR | 441 |
| ILNLLSMV | 442 |
| ILPRSPAF | 443 |
| IPFSWSRF | 444 |
| IQNLLSMV | 445 |
| ISSGL | 446 |
| ISSGLL | 447 |
| ISSGLLS | 448 |
| ISSGLLSGRSANI | 449 |
| ISSGLLSGRSANP | 450 |
| ISSGLLSGRSANPRG | 451 |
| ISSGLLSGRSDDH | 452 |
| ISSGLLSGRSDIH | 453 |
| ISSGLLSGRSDNH | 454 |
| ISSGLLSGRSDNI | 455 |
| ISSGLLSGRSDNP | 456 |
| ISSGLLSGRSDQH | 457 |
| ISSGLLSGRSDTH | 458 |
| ISSGLLSGRSDYH | 459 |
| ISSGLLSGRSGNH | 460 |
| ISSGLLSGRSNI | 461 |
| ISSGLLSGRSNIG | 462 |
| ISSGLLSGRSNIGS | 463 |
| ISSGLLSS | 464 |
| ISSGLLSSGGSGGSLSGRSDNH | 465 |
| ISSGLLSSGGSGGSLSGRSGNH | 466 |
| ISSGLSS | 467 |
| IVSRSA | 468 |
| KGLTGRSDRHQA | 469 |
| KGPKVKVVTL | 470 |
| KNLYGRSENNGN | 471 |
| KRMPVQFL | 472 |
| LAAPLGLL | 473 |
| LAHG | 474 |
| LAHGL | 475 |
| LAHGLF | 476 |
| LAPLGLQRR | 477 |
| LARAG | 478 |
| LARAGI | 479 |
| LARAGL | 480 |
| LKAAPRWA | 481 |
| LKAAPRWF | 482 |
| LKAAPVWA | 483 |
| LKAAPVWF | 484 |
| LKGRSYYY | 485 |
| LLAPSHRA | 486 |
| LLEALRAL | 487 |
| LLESLRAL | 488 |
| LLLPAHGG | 489 |
| LLLPLLGS | 490 |
| LLNALRAL | 491 |
| LLNSLRAL | 492 |
| LLQALRAL | 493 |
| LLQSLRAL | 494 |
| LLSALRAL | 495 |
| LLSSLRAL | 496 |
| LNGRSDNH | 497 |
| LPAGLLL | 498 |
| LPAGLLLR | 499 |
| LPAHLVLL | 530 |
| LPAHLVLV | 531 |
| LPGGLSPW | 532 |
| LPSHLVLL | 533 |
| LPSHLVLV | 534 |
| LPTFV | 535 |
| LPTFVH | 536 |
| LPTFVHL | 537 |
| LRSGW | 538 |
| LSGR | 539 |
| LSGRS | 540 |
| LSGRSA | 541 |
| LSGRSALAHGLF | 542 |
| LSGRSAN | 543 |
| LSGRSANI | 544 |
| LSGRSANP | 545 |
| LSGRSD | 546 |
| LSGRSDD | 547 |
| LSGRSDDH | 548 |
| LSGRSDI | 549 |
| LSGRSDIH | 550 |
| LSGRSDN | 551 |
| LSGRSDNH | 552 |
| LSGRSNI | 553 |
| LSGRSDNHGGAVGLLAPP | 554 |
| LSGRSDNHGGSGGSISSGLLSS | 555 |
| LSGRSDNHGGSGGSQNQALRMA | 556 |
| LSGRSDNHGGVHMPLGFLGP | 557 |
| LSGRSDNI | 558 |
| LSGRSDNP | 559 |
| LSGRSDQ | 560 |
| LSGRSDQH | 561 |
| LSGRSDT | 562 |
| LSGRSDTH | 563 |
| LSGRSDY | 564 |
| LSGRSDYH | 565 |
| LSGRSENH | 566 |
| LSGRSG | 567 |
| LSGRSGN | 568 |
| LSGRSGNH | 569 |
| LSGRSGNHGGSGGSISSGLLSS | 570 |
| LSGRSGNHGGSGGSQNQALRMA | 571 |
| LSGRSGNP | 572 |
| LSGRSVTQ | 573 |
| LSQARWRK | 574 |
| LTFPTYIF | 575 |
| LTFPTYWF | 576 |
| LTGRSDRH | 577 |
| LTGRSGA | 578 |
| LTGRSGA | 579 |
| LYAAPRWA | 580 |
| LYAAPRWF | 581 |
| LYAAPVWA | 582 |
| LYAAPVWF | 583 |
| LYGRSENN | 584 |
| MDAFLESS | 585 |
| MGLFSEAG | 586 |
| MGPWFM | 587 |
| MIAPVAYR | 588 |
| MLRSGW | 589 |
| MLRSGWR | 590 |
| MLRSGWRG | 591 |
| MLRSGWRL | 592 |
| MLRSGWRS | 593 |
| MTFPTYIF | 594 |
| MTFPTYWF | 595 |
| NHRIGRSDNHRR | 596 |
| NMPSFKLVTG | 597 |
| NTLSGRSENHSG | 598 |
| NTLSGRSGNHGS | 599 |
| NZPRVRLVLP | 603 |
| PAGLWLDP | 604 |
| PAGRR | 605 |
| PAGRRS | 606 |
| PAGRRSL | 607 |
| PASLWYTQ | 608 |
| PESRRWMP | 609 |
| PFHLSR | 610 |
| PHGFFQ | 611 |
| PLARAGI | 612 |
| PLARAGL | 613 |
| PLGL | 614 |
| PLGLAG | 615 |
| PLGLWA | 616 |
| PLGVRGK | 617 |
| PLTGRSGG | 618 |
| PLTGRSGGGGSS | 619 |
| PPLGR | 620 |
| PPPDMKLFPG | 621 |
| PPPEVRSFSV | 622 |
| PPPVLKLLEW | 623 |
| PPSIARSDNLAN | 624 |
| PQHRIVSF | 625 |
| PRFKIIGG | 626 |
| PRFRIIGG | 627 |
| PRPFVKSVDQ | 628 |
| PRQV | 629 |
| PRSF | 630 |
| PSPPVKMMPE | 631 |
| PTNGGSGGSS | 632 |
| RVPKVKVMLD | 633 |
| SAGFSLPA | 634 |
| SAPAVESE | 635 |
| SAPYFRMMDM | 636 |
| SARGPSRW | 637 |
| SCGPPLGR | 638 |
| SCSPPLGR | 639 |
| SGGPLGVR | 640 |
| SGGPPLGR | 641 |
| SGPPLGR | 642 |
| SGRS | 643 |
| SGRSA | 644 |
| SGRSAN | 645 |
| SGRSANI | 646 |
| SGRSANP | 647 |
| SGRSANPRG | 648 |
| SGRSD | 649 |
| SGRSDD | 650 |
| SGRSDDH | 651 |
| SGRSDI | 652 |
| SGRSDIH | 653 |
| SGRSDN | 654 |
| SGRSDNI | 655 |
| SGRSDNP | 656 |
| SGRSDQ | 657 |
| SGRSDQH | 658 |
| SGRSDT | 659 |
| SGRSDTH | 660 |
| SGRSDY | 661 |
| SGRSDYH | 662 |
| SGRSG | 663 |
| SGRSGN | 664 |
| SGRSGNH | 665 |
| SGSPPLGR | 666 |
| SIARSDNL | 667 |
| SISSGLLSGRSDNI | 668 |
| SNPFKY | 669 |
| SPLPLRVP | 670 |
| SPLPLRVP | 671 |
| SPLTGRSG | 672 |
| SPPLGR | 673 |
| SRRVP | 674 |
| SRRVPL | 675 |
| SRRVPV | 676 |
| SSGPPLGR | 677 |
| SSPPLGR | 678 |
| SSRGPAYL | 679 |
| SSRHRRALD | 680 |
| SSSFDKGKYKKGDDA | 681 |
| SSSFDKGKYKRGDDA | 682 |
| SSSPPLGR | 683 |
| STFPFGMF | 684 |
| STVFHM | 685 |
| SVHHLI | 656 |
| SVSGLLSH | 687 |
| SVSGLLSS | 688 |
| SVSGLRSH | 689 |
| SVSGLRSS | 690 |
| TARG | 691 |
| TARGP | 692 |
| TARGPALAHGLF | 693 |
| TARGPS | 694 |
| TARGPSF | 695 |
| TARGPSFK | 696 |
| TARGPSW | 697 |
| TARGPSW | 698 |
| TARGPVPRQV | 699 |
| TFVH | 700 |
| TGLSGRSVTQTS | 701 |
| TGRGPSWV | 702 |
| TLRLGRSDNNKN | 703 |
| TLSGLRSP | 704 |
| TSGRSANP | 705 |
| TSGRSGNP | 706 |
| TSLSGRSANPRG | 707 |
| TSLSGRSGNPRG | 708 |
| TSSGLRSP | 709 |
| TSTSGRSANPRG | 710 |
| TSTSGRSANPRGGGAVGLLAPP | 711 |
| TSTSGRSANPRGGGVHMPLGFLG | 712 |
| P | |
| TSTSGRSGNPRG | 713 |
| TVSGLRSP | 714 |
| MVLGRSLL | 715 |
| SPRSIMLA | 716 |
| SMLRSMPL | 717 |
| GLSGRSDNHGGAVGLLAPP | 718 |
| GLSGRSDNHGGVHMPLGFLGP | 719 |

In some embodiments, the CM comprises or consists of a combination, a C-terminal truncation variant, or an N-terminal truncation variant of the example sequences discussed above. Truncation variants of the aforementioned amino acid sequences that are suitable for use in a CM are any that retain the recognition site for the corresponding protease. These include C-terminal and/or N-terminal truncation variants comprising at least 3 contiguous amino acids of the above-described amino acid sequences, or at least 4, or at least 5, or at least 6, or at least 7 amino acids of the foregoing amino acid sequences that retain a recognition site for a protease. In certain embodiments, the truncation variant of the above-described amino acid sequences is an amino acid sequence corresponding to any of the above, but that is C- and/or N-terminally truncated by 1 to about 10 amino acids, 1 to about 9 amino acids, 1 to about 8 amino acids, 1 to about 7 amino acids, 1 to about 6 amino acids, 1 to about 5 amino acids, 1 to about 4 amino acids, or 1 to about 3 amino acids, and which: (1) has at least three amino acid residues; and (2) retains a recognition site for a protease. In some of the foregoing embodiments, the truncated CM is an N-terminally truncated CM. In some embodiments, the truncated CM is a C-terminally truncated CM. In some embodiments, the truncated C is a C- and an N-terminally truncated CM.

In some embodiments, the CM comprises a total of 3 amino acids to 25 amino acids. In some embodiments, the CM comprises a total of 3 to 25, 3 to 20, 3 to 15, 3 to 10, 3 to 5, 5 to 25, 5 to 20, 5 to 15, 5 to 10, 10 to 25, 10 to 20, 10 to 15, 15 to 25, 15 to 20, or 20 to 25 amino acids.

In some embodiments, the CM is specifically cleaved by at least a protease at a rate of about 0.001-1500×10⁴ M⁻¹S⁻¹ or at least 0.001, 0.005, 0.01, 0.05, 0.1, 0.5, 1, 2.5, 5, 7.5, 10, 15, 20, 25, 50, 75, 100, 125, 150, 200, 250, 500, 750, 1000, 1250, or 1500×10⁴ M⁻¹S⁻¹. The rate may be measured as substrate cleavage kinetics (k_{cat}/Kₘ) as disclosed in WO2016118629.

In some embodiments of any of the activatable cytokine constructs described herein, the CM comprise a total of about 3 amino acids to about 25 amino acids. In some embodiments, the CM comprise a total of about 3 amino acids to about 25 amino acids, about 3 amino acids to about 20 amino acids, about 3 amino acids to about 15 amino acids, about 3 amino acids to about 10 amino acids, about 3 amino acids to about 5 amino acids, about 5 amino acids to about 25 amino acids, about 5 amino acids to about 20 amino acids, about 5 amino acids to about 15 amino acids, about 5 amino acids to about 10 amino acids, about 10 amino acids to about 25 amino acids, about 10 amino acids to about 20 amino acids, about 10 amino acids to about 15 amino acids, about 15 amino acids to about 25 amino acids, about 15 amino acids to about 20 amino acids, or about 20 amino acids to about 25 amino acids.

In some embodiments, the ACC comprises multiple CMs that comprise substrates for different proteases. In some embodiments, the CM1 and the CM2 in a dimer construct comprise substrates for different proteases. In some embodiments, the CM1 and the CM2 in a dimer construct comprise substrates for the same protease.

An ACC, or the first and second monomer constructs of an ACC that is a dimer complex, may comprise one or more additional components including one or more linkers, and the like. In some embodiments, the first monomer can include a linker disposed between the CP and the CM. In some embodiments, the CP and the CM directly abut each other.

In some embodiments, in an ACC that is a dimer complex, the first monomer can include a linker disposed between the CP1 and the CM1. In some embodiments, the CP1 and the CM1 directly abut each other in the first monomer. In some embodiments, the first monomer comprises a linker disposed between the CM1 and the DD1. In some embodiments, the linker has a total length of 1 amino acid to about 15 amino acids. In some embodiments, the CM1 and the DD1 directly abut each other in the first monomer. In some embodiments, the CM and any linkers disposed between the CP1 and DD1 have a combined total length of 3 to 15 amino acids, or 3 to 10 amino acids, or 3 to 7 amino acids.

In some embodiments, the second monomer comprises a linker disposed between the CP2 and the CM2. In some embodiments, the CP2 and the CM2 directly abut each other in the second monomer. In some embodiments, the second monomer comprises a linker disposed between the CM2 and the DD2. In some embodiments, the linker has a total length of 1 amino acid to about 15 amino acids. In some embodiments, the linker comprises a sequence of G; GG; or GGGS (SEQ ID NO: 2). In some embodiments, the CM2 (e.g., any of the cleavable moieties described herein) and the DD2 (e.g., any of the DDs described herein) directly abut each other in the second monomer. In some embodiments, the CM and any linkers disposed between the CP2 and DD2 have a combined total length of 3 to 15 amino acids, or 3 to 10 amino acids, or 3 to 7 amino acids.

### Additional Masking Moieties (MMs)

In some embodiments, the ACC herein may comprise one or more MM in addition to the MM described above. In some embodiments, the additional MM interacts with the cytokine polypeptide, thus reducing or inhibiting the interaction between the cytokine polypeptide and its binding partner. In some embodiments, the additional MM comprises at least a partial or complete amino acid sequence of a naturally occurring binding partner of the cytokine polypeptide. For example, the additional MM may be a fragment of a naturally occurring binding partner. The fragment may retain no more than 95%, 90%, 80%, 75%, 70%, 60%, 50%, 40%, 30%, 25%, or 20% nucleic acid or amino acid sequence homology to the naturally occurring binding partner. The term “naturally occurring” as used herein as applied to an object refers to the fact that an object can be found in nature. For example, a polypeptide or polynucleotide sequence that is present in an organism (including viruses) that can be isolated from a source in nature and that has not been intentionally modified by man in the laboratory or otherwise is naturally occurring.

In some embodiments, the additional MM comprises an amino acid sequence that is not naturally occurring or does not contain the amino acid sequence of a naturally occurring binding partner. In certain embodiments, the MM is not a natural binding partner of the cytokine polypeptide. The additional MM may be a modified binding partner for the cytokine polypeptide which contains amino acid changes that decrease affinity and/or avidity of binding to the cytokine polypeptide. In some embodiments the additional MM contains no or substantially no nucleic acid or amino acid homology to the cytokine polypeptide's natural binding partner. In other embodiments the additional MM is no more than 5%, 10%, 15%, 20%, 25%, 30%, 35%, 40%, 45%, 50%, 55%, 60%, 65%, 70%, 75%, or 80% similar to the natural binding partner of the cytokine polypeptide.

In some embodiments, the additional MM does not specifically bind to the cytokine polypeptide, but still interferes with cytokine polypeptide's binding to its binding partner through non-specific interactions such as steric hindrance (a “steric mask”). For example, the additional MM may be positioned in the ACC such that the tertiary or quaternary structure of the ACC allows the additional MM to mask the cytokine polypeptide through charge-based interaction, thereby holding the additional MM in place to interfere with binding partner access to the cytokine polypeptide.

In some embodiments, the additional MM has a dissociation constant for binding to the cytokine polypeptide that is no more than the dissociation constant of the cytokine polypeptide to the binding partner. In some embodiments, the additional MM does not interfere or compete with the cytokine polypeptide for binding to the binding partner in a cleaved state.

The structural properties of the MMs may be selected according to factors such as the minimum amino acid sequence required for interference with protein binding to binding partner, the binding partner protein-protein binding pair of interest, the size of the cytokine polypeptide, the presence or absence of linkers, and the like.

In some embodiments, the additional MM is unique for the coupled cytokine polypeptide. Examples of additional MMs include MMs that were specifically screened to bind a binding domain of the cytokine polypeptide or fragment thereof (e.g., affinity masks). Methods for screening MMs to obtain MMs unique for the cytokine polypeptide and those that specifically and/or selectively bind a binding domain of a binding partner are provided herein and can include protein display methods.

In some embodiments, the additional MM is a polypeptide of about 2 to 50 amino acids in length. For example, the additional MM may be a polypeptide of from 2 to 40, from 2 to 30, from 2 to 20, from 2 to 10, from 5 to 15, from 10 to 20, from 15 to 25, from 20 to 30, from 25 to 35, from 30 to 40, from 35 to 45, from 40 to 50 amino acids in length. For example, the additional MM may be a polypeptide with 2, 3, 4, 5, 6, 7, 8, 9, 10, 11, 12, 13, 14, 15, 16, 17, 18, 19,20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some examples, the additional MM may be a polypeptide of more than 50 amino acids in length, e.g., 100, 200, 300, 400, 500, 600, 700, 800, or more amino acids.

In some embodiments, in an inactive state of the ACC with a cytokine polypeptide and an interfering MM, in the presence of the binding partner of a cytokine polypeptide, there is no binding or substantially no binding of the cytokine polypeptide to the binding partner, or no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding of the cytokine polypeptide to its binding partner, as compared to the binding of an counterpart antibody without the interfering MM, for at least 0.1, 0.5, 1, 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, or 12 months when measured in vitro immunoabsorbant assay, e.g., as described in US20200308243A1.

The binding affinity of the cytokine polypeptide towards the binding partner with an interfering MM may be at least 5, 10, 25, 50, 100, 250, 500, 1,000, 2,500, 5,000, 10,000, 50,000, 100,000, 500,000, 1,000,000, 5,000,000, 10,000,000, 50,000,000 times lower than the binding affinity of the cytokine polypeptide towards its binding partner without an interfering MM, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times lower than the binding affinity of the cytokine polypeptide towards its binding partner when there is no interfering MM.

The dissociation constant of the MM towards the cytokine polypeptide it masks, may be greater than the dissociation constant of the cytokine polypeptide towards the binding partner. The dissociation constant of the MM towards the masked cytokine polypeptide may be at least 5, 10, 25, 50, 100, 250, 500, 1,000, 2,500, 5,000, 10,000, 100,000, 1,000,000 or even 10,000,000 times greater than the dissociation constant of the cytokine polypeptide towards the binding partner. Conversely, the binding affinity of the MM towards the masked cytokine polypeptide may be lower than the binding affinity of the cytokine polypeptide towards the binding partner. The binding affinity of MM towards the cytokine polypeptide may be at least 5, 10, 25, 50, 100, 250, 500, 1,000, 2,500, 5,000, 10,000, 100,000, 1,000,000 or even 10,000,000 times lower than the binding affinity of the cytokine polypeptide towards the binding partner.

In some embodiments, the additional MM contains genetically encoded or genetically non-encoded amino acids. Examples of genetically non-encoded amino acids include but are not limited to D-amino acids, 3-amino acids, and T-amino acids. In specific embodiments, the MMs contain no more than 50%, 40%, 30%, 20%, 15%, 10%, 5% or 1% of genetically non-encoded amino acids.

In some embodiments, once released from the ACC and in a free state, the additional MM has a biological activity or a therapeutic effect, such as binding capability. For example, the free peptide may bind with the same or a different binding partner. In certain embodiments, the free MM exerts a therapeutic effect, providing a secondary function to the compositions disclosed herein. In some embodiments, once uncoupled from the ACC and in a free state, the MM may advantageously not exhibit biological activity. For example, in some embodiments the MM in a free state does not elicit an immune response in the subject.

Suitable additional MMs may be identified and/or further optimized through a screening procedure from a library of candidate ACC having variable MMs. For example, a cytokine polypeptide and a CM may be selected to provide for a desired enzyme/target combination, and the amino acid sequence of the additional MM can be identified by the screening procedure described below to identify a MM that provides for a switchable phenotype. For example, a random peptide library (e.g., of peptides comprising 2 to 40 amino acids or more) may be used in the screening methods disclosed herein to identify a suitable MM.

Examples of additional MM include polypeptides that bind to IL-15 and/or IL-2, e.g., any one of SEQ ID NOs: 358-374.

### Linkers

In some embodiments of any of the ACCs described herein, one or more linkers (e.g., flexible linkers) are introduced into the activatable cytokine construct to provide flexibility at one or more of the junctions between domains, between moieties, between moieties and domains, or at any other junctions where a linker would be beneficial. In some embodiments, where the ACC is provided as a conformationally constrained construct, a flexible linker is inserted to facilitate formation and maintenance of a structure in the uncleaved activatable cytokine construct. Any of the linkers described herein can provide the desired flexibility to facilitate the inhibition of the binding of a binding partner (e.g., a receptor of a cytokine), or to facilitate cleavage of a CM by a protease. In some embodiments, linkers are included in the ACC that are all or partially flexible, such that the linker can include a flexible linker as well as one or more portions that confer less flexible structure to provide for a desired ACC. Some linkers may include cysteine residues, which may form disulfide bonds and reduce flexibility of the construct. In some embodiments, reducing the length of the linkers or Linking Region reduces the activity of the mature cytokine polypeptide in the ACCs. In most instances, linker length is determined by counting, in a N- to C-direction, the number of amino acids from the N-terminus of the linker adjacent to the C-terminal amino acid of the preceding component, to the C-terminus of the linker adjacent to the N-terminal amino acid of the following component (i.e., where the linker length does not include either the C-terminal amino acid of the preceding component or the N-terminal amino acid of the following component). In embodiments in which a linker is employed at the N-terminus of a DD that comprises an Fc domain, linker length is determined by counting the number of amino acids from the N-terminus of the linker adjacent to the C-terminal amino acid of the preceding component to C-terminus of the linker adjacent to the first cysteine of an Fc hinge region (i.e., where the linker length does not include the C-terminal amino acid of the preceding component or the first cysteine of the Fc hinge region).

In some embodiments, ACCs of the present disclosure include a stretch of amino acids between the CP and the proximal point of interaction between the dimerization domains (see the example in FIG. 4). That stretch of amino acids may be referred to as a Linking Region (LR). As used herein, the term “Linking Region” or “LR” refers to the stretch of amino acid residues between the C-terminus of the cytokine and the amino acid residue that is N-terminally adjacent to the proximal point of interaction between the dimerization domains (i.e., the linking region does not include the C-terminal amino acid of the cytokine or the N-terminal amino acid of the DD that forms the proximal point of interaction to the DD of the corresponding second monomer). For example, when the DDs are a pair of Fc domains, the linking region is the stretch of amino acid residues between the C-terminus of the cytokine and the first N-terminal cysteine residue that participates in the disulfide linkage of the Fc (e.g., Cysteine 226 of an IgG1 or IgG4 Fc domain, according to EU numbering). When the dimerization domain is not a peptide, then the linking region is the stretch of amino acid residues following the C-terminus of the cytokine until the last amino acid. For example, when the DDs are a biotin-streptavidin pair, the linking region of the biotin-containing monomer is the stretch of amino acid residues between the C-terminus of the cytokine and the biotin molecule, and the linking region of the streptavidin-containing monomer is the stretch of amino acid residues between the C-terminus of the cytokine and the streptavidin molecule. In some aspects, the Linking Region may comprise no more than 24, 18, 14, 12, 11, 10, 9, 8, 7, 6, 5, or 4 amino acids, e.g., 5 to 14, 7 to 12, 7 to 11, or 8 to 11 amino acids.

In some embodiments, additional amino acid sequences are positioned N-terminally or C-terminally to any of the domains of any of the ACCs. Examples include, but are not limited to, targeting moieties (e.g., a ligand for a receptor of a cell present in a target tissue) and serum half-life extending moieties (e.g., polypeptides that bind serum proteins, such as immunoglobulin (e.g., IgG) or serum albumin (e.g., human serum albumin (HSA)).

In some embodiments of any of the activatable cytokine constructs described herein, a linker can include a total of about 1 amino acid to about 25 amino acids (e.g., about 1 amino acid to about 24 amino acids, about 1 amino acid to about 22 amino acids, about 1 amino acid to about 20 amino acids, about 1 amino acid to about 18 amino acids, about 1 amino acid to about 16 amino acids, about 1 amino acid to about 15 amino acids, about 1 amino acid to about 14 amino acids, about 1 amino acid to about 12 amino acids, about 1 amino acid to about 10 amino acids, about 1 amino acid to about 8 amino acids, about 1 amino acid to about 6 amino acids, about 1 amino acid to about 5 amino acids, about 1 amino acid to about 4 amino acids, about 1 amino acid to about 3 amino acids, about 1 amino acid to about 2 amino acids, about 2 amino acids to about 25 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 15 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 5 amino acids, about 2 amino acids to about 4 amino acids, about 2 amino acids to about 3 amino acids, about 4 amino acids to about 25 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 15 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 4 amino acids to about 5 amino acids, about 5 amino acids to about 25 amino acids, about 5 amino acids to about 24 amino acids, about 5 amino acids to about 22 amino acids, about 5 amino acids to about 20 amino acids, about 5 amino acids to about 18 amino acids, about 5 amino acids to about 16 amino acids, about 5 amino acids to about 15 amino acids, about 5 amino acids to about 14 amino acids, about 5 amino acids to about 12 amino acids, about 5 amino acids to about 10 amino acids, about 5 amino acids to about 8 amino acids, about 5 amino acids to about 6 amino acids, about 6 amino acids to about 25 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 15 amino acids, about 6 amino acids to about 14 amino acids, about 6 amino acids to about 12 amino acids, about 6 amino acids to about 10 amino acids, about 6 amino acids to about 8 amino acids, about 8 amino acids to about 25 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 15 amino acids, about 8 amino acids to about 14 amino acids, about 8 amino acids to about 12 amino acids, about 8 amino acids to about 10 amino acids, about 10 amino acids to about 25 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 15 amino acids, about 10 amino acids to about 14 amino acids, about 10 amino acids to about 12 amino acids, about 12 amino acids to about 25 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 15 amino acids, about 12 amino acids to about 14 amino acids, about 14 amino acids to about 25 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 14 amino acids to about 15 amino acids, about 15 amino acids to about 25 amino acids, about 15 amino acids to about 24 amino acids, about 15 amino acids to about 22 amino acids, about 15 amino acids to about 20 amino acids, about 15 amino acids to about 18 amino acids, about 15 amino acids to about 16 amino acids, about 16 amino acids to about 25 amino acids, about 16 amino acids to about 24 amino acids, about 16 amino acids to about 22 amino acids, about 16 amino acids to about 20 amino acids, about 16 amino acids to about 18 amino acids, about 18 amino acids to about 25 amino acids, about 18 amino acids to about 24 amino acids, about 18 amino acids to about 22 amino acids, about 18 amino acids to about 20 amino acids, about 20 amino acids to about 25 amino acids, about 20 amino acids to about 24 amino acids, about 20 amino acids to about 22 amino acids, about 22 amino acid to about 25 amino acids, about 22 amino acid to about 24 amino acids, or about 24 amino acid to about 25 amino acids).

In some embodiments of any of the ACCs described herein, the linker includes a total of about 1 amino acid, about 2 amino acids, about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 21 amino acids, about 22 amino acids, about 23 amino acids, about 24 amino acids, or about 25 amino acids.

In some embodiments, the ACC does not comprise any linkers between the CP and the DD. Such ACCs may exhibit the most significant reduction in cytokine activity relative to the wild type mature cytokine. Further, a configuration in which there are no linkers between the CP and the DD may still allow effective cleavage of a CM positioned between the CP and the DD. Thus, in some embodiments, the ACC does not comprise any linkers between the CP and the DD, and the CM between the CP and the DD comprises not more than 10, 9, 8, 7, 6, 5, 4, or 3 amino acids. In some embodiments the total number of amino acids in the LR comprises not more than 25 amino acids, e.g., not more than 25, 24, 23, 22, 21, 20, 19, 18, 17, 16, 15, 14, 13, 12, 11, 10, 9, 8, 7, 6, 5, 4, or 3 amino acids, or 3 to 10 amino acids or 5 to 15 amino acids, or 7 to 12 amino acids, or any range or specific number of amino acids selected from the range encompassed by 3 to 25 amino acids.

In some embodiments, a linker is rich in glycine (Gly or G) residues. In some embodiments, the linker is rich in serine (Ser or S) residues. In some embodiments, the linker is rich in glycine and serine residues. In some embodiments, the linker has one or more glycine-serine residue pairs (GS) (e.g., 1, 2, 3, 4, 5, 6, 7, 8, 9, or 10 or more GS pairs). In some embodiments, the linker has one or more Gly-Gly-Gly-Ser (GGGS; SEQ ID NO: 228) sequences (e.g., 1, 2, 3, 4, 5, 6, 7, 8, 9, or 10 or more GGGS sequences). In some embodiments, the linker has one or more Gly-Gly-Gly-Gly-Ser (GGGGS; SEQ ID NO: 216) sequences (e.g., 1, 2, 3, 4, 5, 6, 7, 8, 9, or 10 or more GGGGS sequences). In some embodiments, the linker has one or more Gly-Gly-Ser-Gly (GGSG; SEQ ID NO: 229) sequences (e.g., 1, 2, 3, 4, 5, 6, 7, 8, 9, or 10 or more GGSG sequences).

In some embodiments of any of the ACCs described herein, a linker includes any one of or a combination of one or more of: G, GG, GSSGGSGGSGG (SEQ ID NO: 210), GGGS (SEQ ID NO: 2), GGGSGGGS (SEQ ID NO: 211), GGGSGGGSGGGS (SEQ ID NO: 212), GGGGSGGGGSGGGGS (SEQ ID NO: 213), GGGGSGGGGSGGGGSGGGGSGGGGS (SEQ ID NO: 214), GGGGSGGGGS (SEQ ID NO: 215), GGGGS (SEQ ID NO: 216), GS, GGGGSGS (SEQ ID NO: 217), GGGGSGGGGSGGGGSGS (SEQ ID NO: 218), GGSLDPKGGGGS (SEQ ID NO: 219), PKSCDKTHTCPPCPAPELLG (SEQ ID NO: 220), SKYGPPCPPCPAPEFLG (SEQ ID NO: 221), GKSSGSGSESKS (SEQ ID NO: 222), GSTSGSGKSSEGKG (SEQ ID NO: 223), GSTSGSGKSSEGSGSTKG (SEQ ID NO: 224), and GSTSGSGKPGSGEGSTKG (SEQ ID NO: 225).

Non-limiting examples of linkers can include a sequence that is at least 70% identical (e.g., at least 72%, at least 74%, at least 75%, at least 76%, at least 78%, at least 80%, at least 82%, at least 84%, at least 85%, at least 86%, at least 88%, at least 90%, at least 92%, at least 94%, at least 95%, at least 96%, at least 97%, at least 98%, at least 99%, or 100% identical) to GGGS (SEQ ID NO: 2), GSSGGSGGSGG (SEQ ID NO: 210), GGGGSGGGGSGGGGS (SEQ ID NO: 213), GGGGSGS (SEQ ID NO: 217), GGGGSGGGGSGGGGSGS (SEQ ID NO: 218), GGGGSGGGGSGGGGSGGGGS (SEQ ID NO: 235), GGSLDPKGGGGS (SEQ ID NO: 219), and GSTSGSGKPGSSEGST (SEQ ID NO: 226).

In some embodiments, the linker includes a sequence selected from the group of: GGSLDPKGGGGS (SEQ ID NO: 219), GGGGSGGGGSGGGGSGS (SEQ ID NO: 218), GGGGSGS (SEQ ID NO: 217), GS, (GS)n, (GGS)n, (GSGGS)n (SEQ ID NO: 227) and (GGGS)n (SEQ ID NO: 228), GGSG (SEQ ID NO: 229), GGSGG (SEQ ID NO: 230), GSGSG (SEQ ID NO: 231), GSGGG (SEQ ID NO: 232), GGGSG (SEQ ID NO: 233), GSSSG (SEQ ID NO: 234), GGGGSGGGGSGGGGS (SEQ ID NO: 213), GGGGSGGGGSGGGGSGGGGS (SEQ ID NO: 235), GSTSGSGKPGSSEGST (SEQ ID NO: 226), (GGGGS)n (SEQ ID NO: 216), wherein n is an integer of at least one. In some embodiments, the linker includes a sequence selected from the group consisting of: GGSLDPKGGGGS (SEQ ID NO: 219), GGGGSGGGGSGGGGSGS (SEQ ID NO: 218), GGGGSGS (SEQ ID NO: 217), and GS. In some embodiments of any of the ACCs described herein, the linker includes a sequence selected from the group of: GGGGSGGGGSGGGGS (SEQ ID NO: 213), GGGGSGGGGSGGGGSGGGGS (SEQ ID NO: 235), and GSTSGSGKPGSSEGST (SEQ ID NO: 226). In some embodiments of any of the activatable cytokine constructs described herein, the linker includes a sequence selected from the group of: GGGGSGGGGSGGGGS (SEQ ID NO: 213) or GGGGS (SEQ ID NO: 216). In some embodiments, the linker comprises a sequence of GGGS (SEQ ID NO: 2). In some embodiments, the linker comprises a single glycine residue (G), or a sequence of two glycine residues (GG).

In some embodiments, an ACC can include one, two, three, four, five, six, seven, eight, nine, or ten linker sequence(s) (e.g., the same or different linker sequences of any of the exemplary linker sequences described herein or known in the art). In some embodiments, a linker comprises sulfo-SIAB, SMPB, and sulfo-SMPB, wherein the linkers react with primary amines sulfhydryls.

In some aspects, a spacer is employed in a polypeptide or construct of the present disclosure. As used herein, the term “spacer” or “header” refers to an amino acid residue or an amino acid sequence incorporated at a free terminus of the mature ACC, for example between the signal peptide and the N-terminus of the mature ACC. In some aspects, a spacer comprises one or more glutamine (Q) residues. In some aspects, residues in the spacer minimize aminopeptidase and/or exopeptidase action to prevent cleavage of N-terminal amino acids. Illustrative and non-limiting spacer amino acid sequences may comprise or consist of any of the following exemplary amino acid sequences: QGQSGS (SEQ ID NO:375); GQSGS (SEQ ID NO:376); QSGS (SEQ ID NO: 377); SGS; GS; S; QGQSGQG (SEQ ID NO: 378); GQSGQG (SEQ ID NO: 379); QSGQG (SEQ ID NO: 380); SGQG (SEQ ID NO: 381); GQG; QG; G; QGQSGQ (SEQ ID NO: 382); GQSGQ (SEQ ID NO: 383); QSGQ (SEQ ID NO: 384); QGQSG (SEQ ID NO: 385); QGQS (SEQ ID NO: 386); SGQ; GQ; and Q. In some embodiments, spacer sequences are omitted.

In some embodiments of any of the ACCs described herein, the ACC is characterized by a reduction in at least one activity of the CP, or CP1 and/or CP2 if the ACC is a dimer complex, as compared to a control level of the at least one activity of the CP1 and/or CP2. In some embodiments, a control level is the level of the activity for a recombinant CP, or CP1 and/or CP2 (e.g., a commercially available recombinant CP, or CP1 and/or CP2, a recombinant wild type CP, or CP1 and/or CP2, and the like). In some embodiments, a control level is the level of the activity of a cleaved (activated) form of the ACC. In certain embodiments, a control level is the level of the activity of a pegylated CP, or pegylated CP1 and/or CP2.

In some embodiments, the at least one activity is the binding affinity (K_{D}) of the CP, or CP1 and/or the CP2 for its cognate receptor as determined using surface plasmon resonance (e.g., performed in phosphate buffered saline at 25° C.). In certain embodiments, the at least one activity is the level of proliferation of lymphoma cells. In other embodiments, the at least one activity is the level of JAK/STAT/ISGF3 pathway activation in a lymphoma cell. In some embodiments, the at least one activity is a level of SEAP production in a lymphoma cell. In some embodiments, the at least one activity is a level of SEAP production in a cell-based assay using HEK cells. In a further embodiment, the at least one activity of the CP, or CP1 and/or CP2 is level of cytokine-stimulated gene induction using, for example RNAseq methods (see, e.g., Zimmerer et al., Clin. Cancer Res. 14(18):5900-5906, 2008; Hilkens et al., J. Immunol. 171:5255-5263, 2003).

In some embodiments, the ACC is characterized by at least a 2-fold reduction in at least one CP, or CP1 and/or CP2 activity as compared to the control level of the at least one CP, or CP1 and/or CP2 activity. In some embodiments, the ACC is characterized by at least a 5-fold reduction in at least one activity of the CP, or CP1 and/or CP2 as compared to the control level of the at least one activity of the CP, or CP1 and/or CP2. In some embodiments, the ACC is characterized by at least a 10-fold reduction in at least one activity of the CP, or CP1 and/or CP2 as compared to the control level of the at least one activity of the CP, or CP1 and/or CP2. In some embodiments, the ACC is characterized by at least a 20-fold reduction in at least one activity of the CP, or CP1 and/or CP2 as compared to the control level of the at least one activity of the CP, or CP1 and/or CP2. In some embodiments, the ACC is characterized by at least a 30-fold, 40-fold, 50-fold, 60-fold, 70-fold, 80-fold, 90-fold, 100-fold, 500-fold, or 1000-fold reduction in at least one activity of the CP, or CP1 and/or CP2 as compared to the control level of the at least one activity of the CP, or CP1 and/or CP2. In some embodiments, ACC is characterized by at least a 1- to 20-fold reduction, a 200- to 500-fold reduction, a 300- to 500-fold reduction, a 400- to 500-fold reduction, a 500- to 600-fold reduction, a 600- to 700-fold reduction, a 150- to 1000-fold reduction, a 100- to 1500-fold reduction, a 200- to 1500-fold reduction, a 300- to 1500-fold reduction, a 400- to 1500-fold reduction, a 500- to 1500-fold reduction, a 1000- to 1500-fold reduction, a 100- to 1000-fold reduction, a 200- to 1000-fold reduction, a 300- to 1000-fold reduction, a 400- to 1000-fold reduction, a 500- to 1000-fold reduction, a 100- to 500-fold reduction, a 20- to 50-fold reduction, a 30- to 50-fold reduction, a 40- to 50-fold reduction, a 100- to 400-fold reduction, a 200- to 400-fold reduction, or a 300- to 400-fold reduction, a 100- to 300-fold reduction, a 200- to 300-fold reduction, or a 100- to 200-fold reduction in at least one activity of the CP, or CP1 and/or CP2 as compared to the control level of the at least one activity of the CP, or CP1 and/or CP2.

In some embodiments, the ACC is characterized by generating a cleavage product following exposure to the protease(s), wherein the cleavage product comprises the at least one activity of the CP1 and/or CP2. In some embodiments, the at least one activity of the CP1 and/or CP2 is anti-proliferation activity. In some embodiments, the control level is an EC50 value of the wild type mature cytokine, and wherein ratio of EC50 (cleavage product) to EC50 (wild type control level) is less than about 10, or less than about 9, or less than about 8, or less than about 7, or less than about 6, or less than about 5, or less than about 4, or less than about 3, or less than about 2, or less than about 1.5, or equal to about 1. In some embodiments, the EC50 of the cleavage product is approximately the same as the EC50 of the wild type mature cytokine, demonstrating that following cleavage, the activity of the CP1 and/or CP2 is fully recovered, or nearly fully recovered. In some embodiments, the ratio of the EC50 of the cleavage product to the EC50 of the wildtype control is about 1 to about 10, or about 2 to about 8, or about 3 to about 7, or about 4 to about 6, demonstrating good recovery of cytokine activity following protease activation. In some embodiments, the ACC is characterized by having a cleavage product following protease activation, wherein the ratio of the EC50 of the cleavage product to the EC50 of recombinant IL-15 is 1 to about 10, or about 2 to about 8, or about 3 to about 7, or about 4 to about 6, or about 5 to about 7, or about 6, as measured in IL-2/IL-15 responsive HEK293 cells.

In some embodiments, the control level of the at least one activity of the CP, or CP1 and/or CP2 is the activity of the CP, or CP1 and/or CP2 released from the ACC following cleavage of CM, or CM1 and CM2 by the protease(s) (the “cleavage product”). In some embodiments, the control level of the at least one activity of the CP, or CP1 and/or CP2 is the activity of a corresponding wild type mature cytokine (e.g., recombinant wild type mature cytokine).

In some embodiments, incubation of the ACC with the protease yields an activated cytokine product(s), where one or more activities of CP, or CP1 and/or CP2 of the activated cytokine product(s) is greater than the one or more activities of CP, or CP1 and/or CP2 of the intact ACC. In some embodiments, one or more activities of CP, or CP1 and/or CP2 of the activated cytokine product(s) is at least 1-fold greater than the one or more activities of CP, or CP1 and/or CP2 of the ACC. In some embodiments, one or more activities of CP, or CP1 and/or CP2 of the activated cytokine product(s) is at least 2-fold greater than the one or more activities of CP, or CP1 and/or CP2 of the ACC. In some embodiments, one or more activities of CP, or CP1 and/or CP2 of the activated cytokine product(s) is at least 5-fold greater than the one or more activities of CP, or CP1 and/or CP2 of the ACC. In some embodiments, one or more activities of CP, or CP1 and/or CP2 of the activated cytokine product(s) is at least 10-fold greater than the one or more activities of CP, or CP1 and/or CP2 of the ACC. In some embodiments, one or more activities of CP, or CP1 and/or CP2 of the activated cytokine product(s) is at least 20-fold greater than the one or more activities of CP, or CP1 and/or CP2 of the ACC. In some embodiments, one or more activities of CP, or CP1 and/or CP2 of the activated cytokine product(s) is at least 1- to 20-fold greater, 2- to 20-fold greater, 3- to 20-fold greater, 4- to 20-fold greater, 5- to 20-fold greater, 10- to 20-fold greater, 15- to 20-fold greater, 1- to 15-fold greater, 2- to 15-fold greater, 3- to 15-fold greater, 4- to 15-fold greater, 5- to 15-fold greater, 10-to 15-fold greater, 1- to 10-fold greater, 2- to 10-fold greater, 3- to 10-fold greater, 4- to 10-fold greater, 5- to 10-fold greater, 1- to 5-fold greater, 2- to 5-fold greater, 3- to 5-fold greater, 4- to 5-fold greater, 1- to 4-fold greater, 2- to 4-fold greater, 3- to 4-fold greater, 1- to 3-fold greater, 2- to 3-fold greater, or 1- to 2-fold greater than the one or more activities of CP, or CP1 and/or CP2 of the ACC.

In some embodiments, an ACC can include a sequence that is at least 80% (e.g., at least 82%, at least 84%, at least 86%, at least 88%, at least 90%, at least 92%, at least 94%, at least 96%, at least 98%, at least 99%, or 100%) identical to any one of SEQ ID NOs:423-431. In some embodiments, an ACC can include a sequence that is at least 80% (e.g., at least 82%, at least 84%, at least 86%, at least 88%, at least 90%, at least 92%, at least 94%, at least 96%, at least 98%, at least 99%, or 100%) identical to SEQ ID NO: 423. In some embodiments, an ACC can include a sequence that is at least 80% (e.g., at least 82%, at least 84%, at least 86%, at least 88%, at least 90%, at least 92%, at least 94%, at least 96%, at least 98%, at least 99%, or 100%) identical to SEQ ID NO: 424. In some embodiments, an ACC can include a sequence that is at least 80% (e.g., at least 82%, at least 84%, at least 86%, at least 88%, at least 90%, at least 92%, at least 94%, at least 96%, at least 98%, at least 99%, or 100%) identical to SEQ ID NO: 425. In some embodiments, an ACC can include a sequence that is at least 80% (e.g., at least 82%, at least 84%, at least 86%, at least 88%, at least 90%, at least 92%, at least 94%, at least 96%, at least 98%, at least 99%, or 100%) identical to SEQ ID NO: 426. In some embodiments, an ACC can include a sequence that is at least 80% (e.g., at least 82%, at least 84%, at least 86%, at least 88%, at least 90%, at least 92%, at least 94%, at least 96%, at least 98%, at least 99%, or 100%) identical to SEQ ID NO: 427. In some embodiments, an ACC can include a sequence that is at least 80% (e.g., at least 82%, at least 84%, at least 86%, at least 88%, at least 90%, at least 92%, at least 94%, at least 96%, at least 98%, at least 99%, or 100%) identical to SEQ ID NO: 428. In some embodiments, an ACC can include a sequence that is at least 80% (e.g., at least 82%, at least 84%, at least 86%, at least 88%, at least 90%, at least 92%, at least 94%, at least 96%, at least 98%, at least 99%, or 100%) identical to SEQ ID NO: 429. In some embodiments, an ACC can include a sequence that is at least 80% (e.g., at least 82%, at least 84%, at least 86%, at least 88%, at least 90%, at least 92%, at least 94%, at least 96%, at least 98%, at least 99%, or 100%) identical to SEQ ID NO: 430. In some embodiments, an ACC can include a sequence that is at least 80% (e.g., at least 82%, at least 84%, at least 86%, at least 88%, at least 90%, at least 92%, at least 94%, at least 96%, at least 98%, at least 99%, or 100%) identical to SEQ ID NO: 431.

In some aspects, an ACC may include such sequences but either with or without the signal sequences of those sequences. Signal sequences are not particularly limited. Some non-limiting examples of signal sequences include, e.g., MRAWIFFLLCLAGRALA (SEQ ID NO: 343), MALTFALLVALLVLSCKSSCSVG (SEQ ID NO: 344), METDTLLLWVLLLWVPGSTG (SEQ ID NO: 345).

Various exemplary aspects of these activatable cytokine constructs are described below and can be used in any combination in the methods provided herein without limitation. Exemplary aspects of the activatable cytokine constructs and methods of making activatable cytokine constructs are described below.

In some aspects, the ACC includes a CP1 selected from SEQ ID NOs: 402-422, a CM1 selected from SEQ ID Nos: 5-118, 131-209, 251-314, 432-499, 530-599, and 603-719, and a DD1 dimerized with a CP2 selected from SEQ ID NOs: 402-4122, a CM2 selected from SEQ ID Nos: 5-118, 131-209, 251-314, 432-499, 530-599, and 603-719, and a DD2. In some aspects, the ACC may include, between CP1 and CM1 and/or between CM1 and DD1, a linker selected from SEQ ID Nos: 2 and 210-235, 245, or 250, and between CP2 and CM2 and/or between CM2 and DD2, a linker selected from SEQ ID Nos: 2 and 210-235, 245, or 250. In some embodiments, the ACC includes a DD1 and/or a DD2 that has an amino acid sequence that is at least 80% identical (e.g., at least 82%, at least 84%, at least 85%, at least 86%, at least 88%, at least 90%, at least 92%, at least 94%, at least 95%, at least 96%, at least 97%, at least 98%, at least 99%, or 100% identical) to SEQ ID NO: 3 or SEQ ID NO: 4. In some embodiments, the ACC includes a DD1 that has an amino acid sequence that is at least 80% identical (e.g., at least 82%, at least 84%, at least 85%, at least 86%, at least 88%, at least 90%, at least 92%, at least 94%, at least 95%, at least 96%, at least 97%, at least 98%, at least 99%, or 100% identical) to SEQ ID NO: 315 or SEQ ID NO: 316. In some embodiments, the ACC includes a DD2 that has an amino acid sequence that is at least 80% identical (e.g., at least 82%, at least 84%, at least 85%, at least 86%, at least 88%, at least 90%, at least 92%, at least 94%, at least 95%, at least 96%, at least 97%, at least 98%, at least 99%, or 100% identical) to SEQ ID NO: 315 or SEQ ID NO: 316.

### Conjugation to Agents

This disclosure also provides methods and materials for including additional elements in any of the isolated polypeptides and ACCs described herein including, for example, a targeting moiety to facilitate delivery to a cell or tissue of interest, an agent (e.g., a therapeutic agent, an antineoplastic agent), a toxin, or a fragment thereof.

In some embodiments, the ACC is conjugated to a cytotoxic agent, including, without limitation, a toxin (e.g., an enzymatically active toxin of bacterial, fungal, plant, or animal origin, or fragments thereof) or a radioactive isotope. In some embodiments of any of the ACCs described herein, the activatable cytokine construct is conjugated to a cytotoxic agent including, without limitation, a toxin (e.g., an enzymatically active toxin of bacterial, fungal, plant, or animal origin, or fragments thereof), or a radioactive isotope.

Non-limiting exemplary cytotoxic agents that can be conjugated to any of the ACCs described herein include: dolastatins and derivatives thereof (e.g., auristatin E, AFP, monomethyl auristatin D (MMAD), monomethyl auristatin F (MMAF), monomethyl auristatin E (MMAE), desmethyl auristatin E (DMAE), auristatin F, desmethyl auristatin F (DMAF), dolastatin 16 (DmJ), dolastatin 16 (Dpv), auristatin derivatives (e.g., auristatin tyramine, auristatin quinolone), maytansinoids (e.g., DM-1, DM-4), maytansinoid derivatives, duocarmycin, alpha-amanitin, turbostatin, phenstatin, hydroxyphenstatin, spongistatin 5, spongistatin 7, halistatin 1, halistatin 2, halistatin 3, halocomstatin, pyrrolobenzimidazoles (PBI), cibrostatin6, doxaliform, cemadotin analogue (CemCH2-SH), Pseudomonas toxin A (PES8) variant, Pseudomonase toxin A (ZZ-PE38) variant, ZJ-101, anthracycline, doxorubicin, daunorubicin, bryostatin, camptothecin, 7-substituted campothecin, 10, 11-difluoromethylenedioxycamptothecin, combretastatins, debromoaplysiatoxin, KahaMide-F, discodermolide, and Ecteinascidins.

Non-limiting exemplary enzymatically active toxins that can be conjugated to any of the ACCs described herein include: diphtheria toxin, exotoxin A chain from Pseudomonas aeruginosa, ricin A chain, abrin A chain, modeccin A chain, alpha-sarcin, Aleuriies fordii proteins, dianfhin proteins, Phytoiaca americana proteins (e.g., PAPI, PAPII, and PAP-8), Momordica charantia inhibitor, curcin, crotirs, Sapaonaria officinalis inhibitor, geionin, mitogeliin, restrictocin, phenomycin, neomycin, and tricothecenes.

Non-limiting exemplary anti-neoplastics that can be conjugated to any of the ACCs described herein include: adriamycin, cerubidine, bleomycin, alkeran, velban, oncovin, fluorouracil, methotrexate, thiotepa, bisantrene, novantrone, thioguanine, procarabizine, and cytarabine.

Non-limiting exemplary antivirals that can be conjugated to any of the ACCs described herein include: acyclovir, vira A, and symmetrel.

Non-limiting exemplary antifungals that can be conjugated to any of the ACCs described herein include: nystatin.

Non-limiting exemplary conjugatable detection reagents that can be conjugated to any of the ACCs described herein include: fluorescein and derivatives thereof, fluorescein isothiocyanate (FITC).

Non-limiting exemplary antibacterials that can be conjugated to any of the activatable cytokine constructs described herein include: aminoglycosides, streptomycin, neomycin, kanamycin, amikacin, gentamicin, and tobramycin.

Non-limiting exemplary 3beta,16beta,17alpha-trihydroxycholest-5-en-22-one 16-O-(2-O-4-methoxybenzoyl-beta-D-xylopyranosyl)-(1->3)-(2-O-acetyl-alpha-L-arabinopyranoside) (OSW-1) that can be conjugated to any of the activatable cytokine constructs described herein include: s-nitrobenzyloxycarbonyl derivatives of O6-benzylguanine, topoisomerase inhibitors, hemiasterlin, cephalotaxine, homoharringionine, pyrrol obenzodiazepine dimers (PBDs), functionalized pyrrolobenzodiazepenes, calcicheamicins, podophyiitoxins, taxanes, and vinca alkoids.

Non-limiting exemplary radiopharmaceuticals that can be conjugated to any of the activatable cytokine constructs described herein include: ¹²³I, ⁸⁹Zr, ¹²⁵I, ¹³¹I, ⁹⁹mTc, ²⁰¹Tl, ⁶²Cu, ¹⁸F, ⁶⁸Ga, ¹³N, ¹⁵O, ³⁸K, ⁸²Rb, ¹¹¹In, ¹³³Xe, ¹¹C, and ⁹⁹mTc (Technetium).

Non-limiting exemplary heavy metals that can be conjugated to any of the ACCs described herein include: barium, gold, and platinum.

Non-limiting exemplary anti-mycoplasmals that can be conjugated to any of the ACCs described herein include: tylosine, spectinomycin, streptomycin B, ampicillin, sulfanilamide, polymyxin, and chloramphenicol.

Those of ordinary skill in the art will recognize that a large variety of possible moieties can be conjugated to any of the activatable cytokine constructs described herein. Conjugation can include any chemical reaction that will bind the two molecules so long as the ACC and the other moiety retain their respective activities. Conjugation can include many chemical mechanisms, e.g., covalent binding, affinity binding, intercalation, coordinate binding, and complexation. In some embodiments, the preferred binding is covalent binding. Covalent binding can be achieved either by direct condensation of existing side chains or by the incorporation of external bridging molecules. Many bivalent or polyvalent linking agents are useful in conjugating any of the activatable cytokine constructs described herein. For example, conjugation can include organic compounds, such as thioesters, carbodiimides, succinimide esters, glutaraldehyde, diazobenzenes, and hexamethylene diamines. In some embodiments, the activatable cytokine construct can include, or otherwise introduce, one or more non-natural amino acid residues to provide suitable sites for conjugation.

In some embodiments of any of the ACCs described herein, an agent and/or conjugate is attached by disulfide bonds (e.g., disulfide bonds on a cysteine molecule) to the ACC. Since many cancers naturally release high levels of glutathione, a reducing agent, glutathione present in the cancerous tissue microenvironment can reduce the disulfide bonds, and subsequently release the agent and/or the conjugate at the site of delivery.

In some embodiments of any of the ACCs described herein, when the conjugate binds to its target in the presence of complement within the target site (e.g., diseased tissue (e.g., cancerous tissue)), the amide or ester bond attaching the conjugate and/or agent to the linker is cleaved, resulting in the release of the conjugate and/or agent in its active form. These conjugates and/or agents when administered to a subject, will accomplish delivery and release of the conjugate and/or the agent at the target site (e.g., diseased tissue (e.g., cancerous tissue)). These conjugates and/or agents are particularly effective for the in vivo delivery of any of the conjugates and/or agents described herein.

In some embodiments, the linker is not cleavable by enzymes of the complement system. For example, the conjugate and/or agent is released without complement activation since complement activation ultimately lyses the target cell. In such embodiments, the conjugate and/or agent is to be delivered to the target cell (e.g., hormones, enzymes, corticosteroids, neurotransmitters, or genes). Furthermore, the linker is mildly susceptible to cleavage by serum proteases, and the conjugate and/or agent is released slowly at the target site.

In some embodiments of any of the ACCs described herein, the conjugate and/or agent is designed such that the conjugate and/or agent is delivered to the target site (e.g., disease tissue (e.g., cancerous tissue)) but the conjugate and/or agent is not released.

In some embodiments of any of the ACCs described herein, the conjugate and/or agent is attached to an ACC either directly or via a non-cleavable linker. Exemplary non-cleavable linkers include amino acids (e.g., D-amino acids), peptides, or other organic compounds that may be modified to include functional groups that can subsequently be utilized in attachment to ACCs by methods described herein.

In some embodiments of any of the ACCs described herein, an ACC includes at least one point of conjugation for an agent. In some embodiments, all possible points of conjugation are available for conjugation to an agent. In some embodiments, the one or more points of conjugation include, without limitation, sulfur atoms involved in disulfide bonds, sulfur atoms involved in interchain disulfide bonds, sulfur atoms involved in interchain sulfide bonds but not sulfur atoms involved in intrachain disulfide bonds, and/or sulfur atoms of cysteine or other amino acid residues containing a sulfur atom. In such cases, residues may occur naturally in the protein construct structure or are incorporated into the protein construct using methods including, without limitation, site-directed mutagenesis, chemical conversion, or mis-incorporation of non-natural amino acids.

This disclosure also provides methods and materials for preparing an ACC for conjugation. In some embodiments of any of the ACCs described herein, an ACC is modified to include one or more interchain disulfide bonds. For example, disulfide bonds in the ACC can undergo reduction following exposure to a reducing agent such as, without limitation, TCEP, DTT, or β-mercaptoethanol. In some cases, the reduction of the disulfide bonds is only partial. As used herein, the term partial reduction refers to situations where an ACC is contacted with a reducing agent and a fraction of all possible sites of conjugation undergo reduction (e.g., not all disulfide bonds are reduced). In some embodiments, an activatable cytokine construct is partially reduced following contact with a reducing agent if less than 99%, (e.g., less than 98%, 97%, 96%, 95%, 90%, 85%, 80%, 75%, 70%, 65%, 60%, 55%, 50%, 45%, 40%, 35%, 30%, 25%, 20%, 15%, 10% or less than 5%) of all possible sites of conjugation are reduced. In some embodiments, the ACC having a reduction in one or more interchain disulfide bonds is conjugated to a drug reactive with free thiols.

This disclosure also provides methods and materials for conjugating a therapeutic agent to a particular location on an ACC. In some embodiments of any of the ACC described herein, an ACC is modified so that the therapeutic agents can be conjugated to the ACC at particular locations on the ACC. For example, an ACC can be partially reduced in a manner that facilitates conjugation to the ACC. In such cases, partial reduction of the ACC occurs in a manner that conjugation sites in the ACC are not reduced. In some embodiments, the conjugation site(s) on the ACC are selected to facilitate conjugation of an agent at a particular location on the protein construct. Various factors can influence the “level of reduction” of the ACC upon treatment with a reducing agent. For example, without limitation, the ratio of reducing agent to ACC, length of incubation, incubation temperature, and/or pH of the reducing reaction solution can require optimization in order to achieve partial reduction of the ACC with the methods and materials described herein. Any appropriate combination of factors (e.g., ratio of reducing agent to ACC, the length and temperature of incubation with reducing agent, and/or pH of reducing agent) can be used to achieve partial reduction of the ACC (e.g., general reduction of possible conjugation sites or reduction at specific conjugation sites).

An effective ratio of reducing agent to ACC can be any ratio that at least partially reduces the ACC in a manner that allows conjugation to an agent (e.g., general reduction of possible conjugation sites or reduction at specific conjugation sites). In some embodiments, the ratio of reducing agent to ACC will be in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1.

An effective incubation time and temperature for treating an ACC with a reducing agent can be any time and temperature that at least partially reduces the ACC in a manner that allows conjugation of an agent to an ACC (e.g., general reduction of possible conjugation sites or reduction at specific conjugation sites). In some embodiments, the incubation time and temperature for treating an ACC will be in a range from about 1 hour at 37° C. to about 12 hours at 37° C. (or any subranges therein).

An effective pH for a reduction reaction for treating an ACC with a reducing agent can be any pH that at least partially reduces the ACC in a manner that allows conjugation of the ACC to an agent (e.g., general reduction of possible conjugation sites or reduction at specific conjugation sites).

When a partially-reduced ACC is contacted with an agent containing thiols, the agent can conjugate to the interchain thiols in the ACC. An agent can be modified in a manner to include thiols using a thiol-containing reagent (e.g., cysteine or N-acetyl cysteine). For example, the ACC can be partially reduced following incubation with reducing agent (e.g., TCEP) for about 1 hour at about 37° C. at a desired ratio of reducing agent to ACC. An effective ratio of reducing agent to ACC can be any ratio that partially reduces at least two interchain disulfide bonds located in the ACC in a manner that allows conjugation of a thiol-containing agent (e.g., general reduction of possible conjugation sites or reduction at specific conjugation sites).

In some embodiments of any of the ACCs described herein, an ACC is reduced by a reducing agent in a manner that avoids reducing any intrachain disulfide bonds. In some embodiments of any of the ACCs described herein, an ACC is reduced by a reducing agent in a manner that avoids reducing any intrachain disulfide bonds and reduces at least one interchain disulfide bond.

In some embodiments of any of the ACCs described herein, the ACC can also include an agent conjugated to the ACC. In some embodiments, the conjugated agent is a therapeutic agent.

In some embodiments, the agent (e.g., agent conjugated to an activatable cytokine construct) is a detectable moiety such as, for example, a label or other marker. For example, the agent is or includes a radiolabeled amino acid, one or more biotinyl moieties that can be detected by marked avidin (e.g., streptavidin containing a fluorescent marker or enzymatic activity that can be detected by optical or calorimetric methods), one or more radioisotopes or radionuclides, one or more fluorescent labels, one or more enzymatic labels, and/or one or more chemiluminescent agents. In some embodiments, detectable moieties are attached by spacer molecules.

In some embodiments, the agent (e.g., cytotoxic agent conjugated to an activatable cytokine construct) is linked to the ACC using a carbohydrate moiety, sulfhydryl group, amino group, or carboxylate group.

In some embodiments of any of the ACCs described herein conjugated to an agent, the agent (e.g., cytotoxic agent conjugated to an activatable cytokine construct) is conjugated to the ACC via a linker and/or a CM (also referred to as a cleavable sequence). In some embodiments, the agent (e.g., cytotoxic agent conjugated to an activatable cytokine construct) is conjugated to a cysteine or a lysine in the ACC. In some embodiments, the agent (e.g., cytotoxic agent conjugated to an activatable cytokine construct) is conjugated to another residue of the ACC, such as those residues disclosed herein. In some embodiments, the linker is a thiol-containing linker. In some embodiments, the linker is a non-cleavable linker. Some non-limiting examples of cleavable moieties and linkers are provided in Table 2.

TABLE 2

| Types of CMs | Amino Acid Sequence |
|---|---|
| Plasmin CMs | |
| Pro-urokinase | PRFKIIGG (SEQ ID NO: 626) |
| | PRFRIIGG (SEQ ID NO: 627) |
| TGFβ | SSRHRRALD (SEQ ID NO: 680) |
| Plasminogen | RKSSIIIRMRDVVL (SEQ ID NO: 261) |
| Staphylokinase | SSSFDKGKYKKGDDA (SEQ ID NO: 681) |
| | SSSFDKGKYKRGDDA (SEQ ID NO: 682) |
| Factor Xa CMs | IEGR (SEQ ID NO: 441) |
| | IDGR (SEQ ID NO: 440) |
| | GGSIDGR (SEQ ID NO: 104) |
| MMP CMs | |
| Gelatinase A | PLGLWA (SEQ ID NO: 616) |
| Collagenase CMs | |
| Calf skin collagen (α1(I) chain) | GPQGIAGQ (SEQ ID NO: 147) |
| Calf skin collagen (α2(I) chain) | GPQGLLGA (SEQ ID NO: 148) |
| Bovine cartilage collagen (α1(II) chain) | GIAGQ (SEQ ID NO: 107) |
| Human liver collagen (α1(III) chain) | GPLGIAGI (SEQ ID NO: 115) |
| Human α₂M | GPEGLRVG (SEQ ID NO: 114) |
| Human PZP | YGAGLGVV (SEQ ID NO: 308) |
| | AGLGVVER (SEQ ID NO: 16) |
| | AGLGISST (SEQ ID NO: 15) |
| Rat α1M | EPQALAMS (SEQ ID NO: 85) |
| | QALAMSAI (SEQ ID NO: 190) |
| Rat α₂M | AAYHLVSQ (SEQ ID NO: 11) |
| | MDAFLESS (SEQ ID NO: 585) |
| Rat α1I₃(2J) | ESLPVVAV (SEQ ID NO: 88) |
| Rat α1I₃(27J) | SAPAVESE (SEQ ID NO: 635) |
| Human fibroblast collagenase | DVAQFVLT (SEQ ID NO: 76) |
| (autolytic cleavages) | VAQFVLT (SEQ ID NO: 313) |
| | VAQFVLTE (SEQ ID NO: 292) |
| | AQFVLTEG (SEQ ID NO: 38) |
| | PVQPIGPQ (SEQ ID NO: 189) |

Those of ordinary skill in the art will recognize that a large variety of possible moieties can be coupled to the ACCs of the disclosure. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference). In general, an effective conjugation of an agent (e.g., cytotoxic agent) to an ACC can be accomplished by any chemical reaction that will bind the agent to the ACC while also allowing the agent and the ACC to retain functionality.

In some embodiments of any of the ACCs conjugated to an agent, a variety of bifunctional protein-coupling agents can be used to conjugate the agent to the ACC including, without limitation, N-succinimidyl-3-(2-pyridyldithiol) propionate (SPDP), iminothiolane (IT), bifunctional derivatives of imidoesters (e.g., dimethyl adipimidate HCL), active esters (e.g., disuccinimidyl suberate), aldehydes (e.g., glutareldehyde), bis-azido compounds (e.g., bis (p-azidobenzoyl) hexanediamine), bis-diazonium derivatives (e.g., bis-(p-diazoniumbenzoyl)-ethylenediamine), diisocyanates (e.g., tolyene 2,6-diisocyanate), and bis-active fluorine compounds (e.g., 1,5-difluoro-2,4-dinitrobenzene). For example, a ricin immunotoxin can be prepared as described in Vitetta et al., Science 238: 1098 (1987). In some embodiments, a carbon-14-labeled 1-isothiocyanatobenzyl-3-methyldiethylene triaminepentaacetic acid (MX-DTPA) chelating agent can be used to conjugate a radionucleotide to the ACC. (See, e.g., WO94/11026).

Suitable linkers and CMs are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an ACC by way of an oligopeptide linker. In some embodiments, suitable linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (N-hydroxysulfo-succinimide: Pierce Chem. Co., Cat. #24510) conjugated to EDC. Additional linkers include, but are not limited to, SMCC, sulfo-SMCC, SPDB, or sulfo-SPDB.

The CMs and linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically-hindered disulfide bond and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

In some embodiments of any of the ACCs, an agent can be conjugated to the ACC using a modified amino acid sequence included in the amino acid sequence of the ACC. By inserting conjugation-enabled amino acids at specific locations within the amino acid sequence of the ACC, the protein construct can be designed for controlled placement and/or dosage of the conjugated agent (e.g., cytotoxic agent). For example, the ACC can be modified to include a cysteine amino acid residue at positions on the first monomer, the second monomer, the third monomer, and/or the fourth monomer that provide reactive thiol groups and does not negatively impact protein folding and/or assembly and does not alter target-binding properties. In some embodiments, the ACC can be modified to include one or more non-natural amino acid residues within the amino acid sequence of the ACC to provide suitable sites for conjugation. In some embodiments, the ACC can be modified to include enzymatically activatable peptide sequences within the amino acid sequence of the ACC.

### Nucleic Acids

Provided herein are nucleic acids including sequences that encode the isolated polypeptide or ACC, or if the ACC is a dimer complex, the first monomer construct (or the protein portion of the first monomer construct) (e.g., any of the first monomers constructs described herein) and the second monomer construct (or the protein portion of the second monomer construct) (e.g., any of the second monomer constructs described herein) of any of the ACCs described herein. In some embodiments, a pair of nucleic acids together encode the first monomer construct (or the protein portion of the first monomer construct) and the second monomer construct (or the protein portion of the second monomer construct). In some embodiments, the nucleic acid sequence encoding the first monomer construct (or the protein portion of the first monomer construct) is at least 70% identical (e.g., at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical) to the nucleic acid sequence encoding the second monomer construct (or the protein portion of the second monomer construct).

In some embodiments, the nucleic acid encoding the protein portion of a first monomer construct encodes a polypeptide comprising the CP1 and CM1 moieties. In some embodiments, the nucleic acid encoding the protein portion of a second monomer encodes a polypeptide comprising the CP2 and CM2 moieties. In some embodiments, a pair of nucleic acids together encode the protein portion of a first monomer construct and the protein portion of the second monomer construct, wherein the protein portions are then conjugated to the DD1 and DD2 moieties, respectively (in a subsequent conjugation step).

In some embodiments, the nucleic acid encoding the first monomer construct encodes a polypeptide comprising the DD1 moiety. In some embodiments, the nucleic acid encoding the second monomer construct encodes a polypeptide comprising the DD2 moiety.

The present disclosure includes a polynucleotide encoding a protein as described herein or a portion thereof, and use of such polynucleotides to produce the proteins and/or for therapeutic purposes. Such polynucleotides may include DNA and RNA molecules (e.g., mRNA, self-replicating RNA, self-amplifying mRNA, etc.) that encode a protein as defined herein. The present disclosure includes compositions comprising such polynucleotides. In some aspects, such compositions are used therapeutically or prophylactically.

Modifications can be introduced into a nucleotide sequence by standard techniques known in the art, such as site-directed mutagenesis and polymerase chain reaction (PCR)-mediated mutagenesis.

### Vectors

Provided herein are vectors and sets of vectors including any of the nucleic acids described herein. One skilled in the art will be capable of selecting suitable vectors or sets of vectors (e.g., expression vectors) for making any of the ACCs described herein, and using the vectors or sets of vectors to express any of the ACCs described herein. For example, in selecting a vector or a set of vectors, the cell must be considered because the vector(s) may need to be able to integrate into a chromosome of the cell and/or replicate in it. Exemplary vectors that can be used to produce an ACC are also described below.

As used herein, the term “vector” refers to a polynucleotide capable of inducing the expression of a recombinant protein (e.g., a first or second monomer) in a cell (e.g., any of the cells described herein). A “vector” is able to deliver nucleic acids and fragments thereof into a host cell, and includes regulatory sequences (e.g., promoter, enhancer, poly(A) signal). Exogenous polynucleotides may be inserted into the expression vector in order to be expressed. The term “vector” also includes artificial chromosomes, plasmids, retroviruses, and baculovirus vectors.

Methods for constructing suitable vectors that include any of the nucleic acids described herein, and suitable for transforming cells (e.g., mammalian cells) are well-known in the art. See, e.g., Sambrook et al., Eds. “Molecular Cloning: A Laboratory Manual,” 2^{nd} Ed., Cold Spring Harbor Press, 1989 and Ausubel et al., Eds. “Current Protocols in Molecular Biology,” Current Protocols, 1993.

Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. A vector can, for example, include sufficient cis-acting elements for expression; other elements for expression can be supplied by the host mammalian cell or in an in vitro expression system. Skilled practitioners will be capable of selecting suitable vectors and mammalian cells for making any of the ACCs described herein.

In some embodiments of any of the ACCs described herein, the ACC is made biosynthetically using recombinant DNA technology and expression in eukaryotic or prokaryotic species.

In some embodiments, the vector includes a nucleic acid encoding the first monomer and the second monomer of any of the ACCs described herein. In some embodiments, the vector is an expression vector.

In some embodiments, a pair of vectors together include a pair of nucleic acids that together encode the first monomer and the second monomer of any of the ACCs described herein. In some embodiments, the pair of vectors is a pair of expression vectors.

### Cells

Also provided herein are host cells (i.e., recombinant or isolated host cells) including any of the vector or sets of vectors described herein or including any of the nucleic acids described herein.

Methods of introducing nucleic acids and vectors (e.g., any of the vectors or any of the sets of vectors described herein) into a cell are known in the art. Non-limiting examples of methods that can be used to introducing a nucleic acid into a cell include: lipofection, transfection, calcium phosphate transfection, cationic polymer transfection, viral transduction (e.g., adenoviral transduction, lentiviral transduction), nanoparticle transfection, and electroporation.

In some embodiments, the introducing step includes introducing into a cell a vector (e.g., any of the vectors or sets of vectors described herein) including a nucleic acid encoding the monomers that make up any of the ACCs described herein.

In some embodiments of any of the methods described herein, the ACC can be produced by any cell, including a prokaryotic cell (e.g., a bacterial cell) or a eukaryotic cell. As used herein, the term “eukaryotic cell” refers to a cell having a distinct, membrane-bound nucleus. Such cells may include, for example, mammalian, insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include a rodent cell (e.g., a mouse cell, a rat cell, a hamster cell, such as Chinese hamster ovary (CHO) cells, or a non-human primate cell, or a human cell, such as human embryonic kidney cells (e.g., HEK293 cells).

In some embodiments, the cell contains the nucleic acid encoding the first monomer and the second monomer of any one of the ACCs described herein. In some embodiments, the cell contains the pair of nucleic acids that together encode the first monomer and the second monomer of any of the ACCs described herein. In some aspects, the nucleic acid encoding the first monomer and the second monomer is integrated into the genomic DNA of the host cell.

### Methods of Producing Activatable Cytokine Constructs

Provided herein are methods of producing any of the ACCs described herein that include: (a) culturing any of the recombinant host cells described herein in a liquid culture medium under conditions sufficient to produce the ACC; and (b) recovering the ACC from the host cell and/or the liquid culture medium.

Methods of culturing cells are well known in the art. Cells can be maintained in vitro under conditions that favor cell proliferation, cell differentiation and cell growth. For example, cells can be cultured by contacting a cell (e.g., any of the cells described herein) with a cell culture medium that includes the necessary growth factors and supplements sufficient to support cell viability and growth.

In some embodiments of any of the methods described herein, the method further includes isolating the recovered ACC. Non-limiting examples of methods of isolation include: ammonium sulfate precipitation, polyethylene glycol precipitation, size exclusion chromatography, ligand-affinity chromatography, ion-exchange chromatography (e.g., anion or cation), and hydrophobic interaction chromatography.

In some embodiments, the present disclosure includes a method of inducing cells to produce a protein portion of a first monomer construct that includes the CP1, the CM1, the MM2, and the CM3, and a protein portion of a second monomer construct that includes the CP2, and the CM2, and optionally the MM2 and the CM4, and subsequently conjugating the protein portions to the DD1 and DD2 moieties, respectively.

Compositions and methods described herein may involve use of non-reducing or partially-reducing conditions that allow disulfide bonds to form between the dimerization domains to form and maintain dimerization of the ACCs.

In some embodiments of any of the methods described herein, the method further includes formulating the isolated ACC into a pharmaceutical composition. Various formulations are known in the art and are described herein. Any of the isolated ACCs described herein can be formulated for any route of administration (e.g., intravenous, intratumoral, subcutaneous, intradermal, oral (e.g., inhalation), transdermal (e.g., topical), transmucosal, or intramuscular).

Also provided herein are ACCs produced by any of the methods described herein. Also provided are compositions (e.g., pharmaceutical compositions) that include any of the ACCs produced by any of the methods described herein. Also provided herein are kits that include at least one dose of any of the compositions (e.g., pharmaceutical compositions) described herein.

In some embodiments, the ACC disclosed herein includes mutants of the cytokines. For example, mutants can be used that have advantageous properties compared to the wild type cytokines, e.g., exhibit less aggregation compared to wild type cytokine polypeptide or control ACC that does not comprise the mutated cytokine polypeptide. In some embodiments, the present disclosure provides a method of producing an ACC comprising: culturing a cell comprising a polynucleotide encoding an ACC herein in a liquid culture medium under conditions sufficient to produce the ACC; purifying the ACC using an affinity chromatography, wherein the purified polypeptide has a purity of at least about 40% monomer; and recovering the ACC from the cell or the liquid culture medium. In some embodiments, the purified polypeptide has a purity of at least 50%, 60%, 70%, 75%, 80%, 85%, 90%, or 95% monomer.

In another aspect, the present disclosure also provides a composition (e.g., a composition produced during the process of making the ACC), in which at least 40%, 50%, 60%, 70%, 75%, 80%, 85%, 90%, or 95% of the purified ACC is in monomer form.

### Methods of Treatment

Provided herein are methods of treating a disease (e.g., a cancer (e.g., any of the cancers described herein)) in a subject including administering a therapeutically effective amount of any of the ACCs described herein to the subject.

As used herein, the term “subject” refers to any mammal. In some embodiments, the subject is a feline (e.g., a cat), a canine (e.g., a dog), an equine (e.g., a horse), a rabbit, a pig, a rodent (e.g., a mouse, a rat, a hamster or a guinea pig), a non-human primate (e.g., a simian (e.g., a monkey (e.g., a baboon, a marmoset), or an ape (e.g., a chimpanzee, a gorilla, an orangutan, or a gibbon)), or a human. In some embodiments, the subject is a human.

In some embodiments, the subject has been previously identified or diagnosed as having the disease (e.g., cancer (e.g., any of the cancers described herein)).

As used herein, the term “treat” includes reducing the severity, frequency or the number of one or more (e.g., 1, 2, 3, 4, or 5) symptoms or signs of a disease (e.g., a cancer (e.g., any of the cancers described herein)) in the subject (e.g., any of the subjects described herein). In some embodiments where the disease is cancer, treating results in reducing cancer growth, inhibiting cancer progression, inhibiting cancer metastasis, or reducing the risk of cancer recurrence in a subject having cancer.

In some embodiments of any of the methods described herein, the disease is a cancer. Also provided herein are methods of treating a subject in need thereof (e.g., any of the exemplary subjects described herein or known in the art) that include administering to the subject a therapeutically effective amount of any of the ACCs described herein or any of the compositions (e.g., pharmaceutical compositions) described herein.

In some embodiments of these methods, the subject has been identified or diagnosed as having a cancer. Non-limiting examples of cancer include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, a lymphoma (e.g., B-cell lymphoma, B-cell non-Hodgkin's lymphoma, Hodgkin's lymphoma, cutaneous T-cell lymphoma), a leukemia (e.g., hairy cell leukemia, chronic lymphocytic leukemia (CLL), acute myeloid leukemia (AML), chronic myeloid leukemia (CML), acute lymphocytic leukemia (ALL)), myelodysplastic syndromes (MDS), Kaposi sarcoma, retinoblastoma, stomach cancer, urothelial carcinoma, lung cancer, renal cell carcinoma, gastric and esophageal cancer, pancreatic cancer, prostate cancer, brain cancer, colon cancer, bone cancer, lung cancer, breast cancer, colorectal cancer, ovarian cancer, nasopharyngeal adenocarcinoma, non-small cell lung carcinoma (NSCLC), squamous cell head and neck carcinoma, endometrial cancer, bladder cancer, cervical cancer, liver cancer, and hepatocellular carcinoma. In some embodiments, the cancer is a lymphoma. In some embodiments, the lymphoma is Burkitt's lymphoma. In some aspects, the subject has been identified or diagnosed as having familial cancer syndromes such as Li Fraumeni Syndrome, Familial Breast-Ovarian Cancer (BRCA1 or BRAC2 mutations) Syndromes, and others. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine.

Exemplary cancers described by the National Cancer Institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

Further exemplary cancers include diffuse large B-cell lymphoma (DLBCL) and mantle cell lymphoma (MCL).

Metastases of the aforementioned cancers can also be treated or prevented in accordance with the methods described herein.

In some embodiments, these methods can result in a reduction in the number, severity, or frequency of one or more symptoms of the cancer in the subject (e.g., as compared to the number, severity, or frequency of the one or more symptoms of the cancer in the subject prior to treatment).

In some embodiments of any of the methods described herein, the methods further include administering to a subject an additional therapeutic agent (e.g., one or more of the therapeutic agents listed in Table 3).

TABLE 3
Additional Therapeutic Agents

| Antibody Trade Name (antibody name) | Target |
|---|---|
| Raptiva ™ (efalizumab) | CD11a |
| Arzerra ™ (ofatumumab) | CD20 |
| Bexxar ™ (tositumomab) | CD20 |
| Gazyva ™ (obinutuzumab) | CD20 |
| Ocrevus ™ (ocrelizumab) | CD20 |
| Rituxan ™ (rituximab) | CD20 |
| Zevalin ™ (ibritumomab tiuxetan) | CD20 |
| Adcetris ™ (brentuximab vedotin) | CD30 |
| Myelotarg ™ (gemtuzumab) | CD33 |
| Mylotarg ™ (gemtuzumab ozogamicin) | CD33 |
| (vadastuximab) | CD33 |
| (vadastuximab talirine) | CD33 |
| Campath ™ (alemtuzumab) | CD52 |
| Lemtrada ™ (alemtuzumab) | CD52 |
| Tactress ™ (tamtuvetmab) | CD52 |
| Soliris ™ (eculizumab) | Complement C5 |
| Ultomiris ™ (ravulizumab) | Complement C5 |
| (olendalizumab) | Complement C5 |
| Yervoy ™ (ipilimumab) | CTLA-4 |
| (tremelimumab) | CTLA-4 |
| Orencia ™ (abatacept) | CTLA-4 |
| Hu5c8 | CD40L |
| (letolizumab) | CD40L |
| Rexomun ™ (ertumaxomab) | CD3/Her2 |
| Erbitux ™ (cetuximab) | EGFR |
| Portrazza ™ (necitumumab) | EGFR |
| Vectibix ™ (panitumumab) | EGFR |
| CH806 | EGFR |
| (depatuxizumab) | EGFR |
| (depatuxizumab mafodotin) | EGFR |
| (futuximab:modotuximab) | EGFR |
| ICR62 (imgatuzumab) | EGFR |
| (laprituximab) | EGFR |
| (losatuxizumab) | EGFR |
| (losatuxizumab vedotin) | EGFR |
| mAb 528 | EGFR |
| (matuzumab) | EGFR |
| (nimotuzumab) | EGFR |
| (tomuzotuximab) | EGFR |
| (zalutumumab) | EGFR |
| MDX-447 | EGFR/CD64 |
| (adecatumumab) | EpCAM |
| Panorex ™ (edrecolomab) | EpCAM |
| Vicinium ™ | EpCAM |
| Synagis ™ (palivizumab) | F protein of RSV |
| ReoPro ™ (abiciximab) | Glycoprotein receptor |
| | IIb/IIIa |
| Herceptin ™ (trastuzumab) | Her2 |
| Herceptin ™ Hylecta | Her2 |
| (trastuzumab; Hyaluronidase) | |
| (trastuzumab deruxtecan) | Her2 |
| (hertuzumab verdotin) | Her2 |
| Kadcyla ™ (trastuzumab emtansine) | Her2 |
| (margetuximab) | Her2 |
| (timigutuzumab) | Her2 |
| Xolair ™ (omalizumab) | IgE |
| (ligelizumab) | IgE |
| (figitumumab) | IGF1R |
| (teprotumumab) | IGF1R |
| Simulect ™ (basiliximab) | IL2R |
| Zenapax ™ (daclizumab) | IL2R |
| Zinbryta ™ (daclizumab) | IL2R |
| Actemra ™ (tocilizumab) | IL-6 receptor |
| Kevzara ™ (sarilumab) | IL-6 receptor |
| (vobarilizumab) | IL-6 receptor |
| Stelara ™ (ustekinumab) | IL-12/IL-23 |
| Tysabri ™ (natalizumab) | Integrinα4 |
| (abrilumab) | Integrinα4 |
| | Jagged 1 or Jagged 2 |
| (fasinumab) | NGF |
| (fulranumab) | NGF |
| (tanezumab) | NGF |
| | Notch, e.g., Notch 1 |
| Pidilizumab | Delta like-1 (PD-1 |
| | pathway inhibitor) |
| Opdivo ® (nivolumab) | PD1 |
| Keytruda ® (pembrolizumab) | PD1 |
| Libtayo ® (cemiplimab) | PD1 |
| BGB-A317 (tislelizumab) | PD1 |
| PDR001 (spartalizumab) | PD1 |
| JNJ-63723283 (cetrelimab) | PD1 |
| TSR042 (dostarlimab) | PD1 |
| AGEN2034 (balstilimab) | PD1 |
| JS001 (toripalimab) | PD1 |
| IOBI308 (sintilimab) | PD1 |
| BCD100 (prolgolimab) | PD1 |
| CBT-501 (genolimzumab | PD1 |
| ABBV181 (budigalimab) | PD1 |
| AK105 | PD1 |
| BI-754091 | PD1 |
| INCSHR-1210 | PD1 |
| MEDI0680 | PD1 |
| MGA012 | PD1 |
| SHR-1210 | PD1 |
| Imfinzi ™ (durvalumab) | PD-L1 |
| Tecentriq ® (atezolizumab) | PD-L1 |
| Bavencio ® (avelumab) | PD-L1 |
| KN035 (envafolimab) | PD-L1 |
| BMS936559 (MDX1105) | PD-L1 |
| BGBA 333 | PD-L1 |
| FAZ053 | PD-L1 |
| LY-3300054 | PD-L1 |
| SH-1316 | PD-L1 |
| AMP-224 | PD-L2 |
| (bavituximab) | Phosphatidylserine |
| huJ591 | PSMA |
| RAV12 | RAAG12 |
| Prolia ™ (denosumab) | RANKL |
| GC1008 (fresolimumab) | TGFbeta |
| Cimzia ™ (Certolizumab Pegol) | TNFα |
| Remicade ™ (infliximab) | TNFα |
| Humira ™ (adalimumab) | TNFα |
| Simponi ™ (golimumab) | TNFα |
| Enbrel ™ (etanercept) | TNF-R |
| (mapatumumab) | TRAIL-R1 |
| Avastin ™ (bevacizumab) | VEGF |
| Lucentis ™ (ranibizumab) | VEGF |
| (brolucizumab) | VEGF |
| (vanucizumab) | VEGF |

### Compositions/Kits

Also provided herein are compositions (e.g., pharmaceutical compositions) including any of the ACCs described herein and one or more (e.g., 1, 2, 3, 4, or 5) pharmaceutically acceptable carriers (e.g., any of the pharmaceutically acceptable carriers described herein), diluents, or excipients.

In some embodiments, the compositions (e.g. pharmaceutical compositions) that include any of the ACCs described herein can be disposed in a sterile vial or a pre-loaded syringe.

In some embodiments, the compositions (e.g. pharmaceutical compositions) that include any of the ACCs described herein can be formulated for different routes of administration (e.g., intravenous, subcutaneous, intramuscular, intraperitoneal, or intratumoral).

In some embodiments, any of the pharmaceutical compositions described herein can include one or more buffers (e.g., a neutral-buffered saline, a phosphate-buffered saline (PBS), amino acids (e.g., glycine), one or more carbohydrates (e.g., glucose, mannose, sucrose, dextran, or mannitol), one or more antioxidants, one or more chelating agents (e.g., EDTA or glutathione), one or more preservatives, and/or a pharmaceutically acceptable carrier (e.g., bacteriostatic water, PBS, or saline).

As used herein, the phrase “pharmaceutically acceptable carrier” refers to any and all solvents, dispersion media, coatings, antibacterial agents, antimicrobial agents, isotonic and absorption delaying agents, and the like, compatible with pharmaceutical administration. Suitable carriers include, but are not limited to: water, saline, ringer's solutions, dextrose solution, and about 5% human serum albumin.

In some embodiments of any of the pharmaceutical compositions described herein, any of the ACCs described herein are prepared with carriers that protect against rapid elimination from the body, e.g., sustained and controlled release formulations, including implants and microencapsulated delivery systems. Biodegradable, biocompatible polymers can be used, e.g., ethylene vinyl acetate, polyanhydrides, polyglycolic acid, collage, polyorthoesters, and polylactic acid. Methods for preparation of such pharmaceutical compositions and formulations are apparent to those skilled in the art.

Also provided herein are kits that include any of the ACCs described herein, any of the compositions that include any of the ACCs described herein, or any of the pharmaceutical compositions that include any of the ACCs described herein. Also provided are kits that include one or more second therapeutic agent(s) selected from Table 3 in addition to an ACC described herein. The second therapeutic agent(s) may be provided in a dosage administration form that is separate from the ACC. Alternatively, the second therapeutic agent(s) may be formulated together with the ACC. In some embodiments, the kit comprises (1) an ACC comprising an amino acid sequence selected from the group consisting of SEQ ID NO: 129 and SEQ ID NOs: 347-356, and (2) a second therapeutic agent selected from Table 3.

Any of the kits described herein can include instructions for using any of the compositions (e.g., pharmaceutical compositions) and/or any of the ACCs described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

### EXAMPLES

The invention is further described in the following examples, which do not limit the scope of the invention described in the claims.

### Example 1: Production of Activatable IL-15 Cytokine Constructs

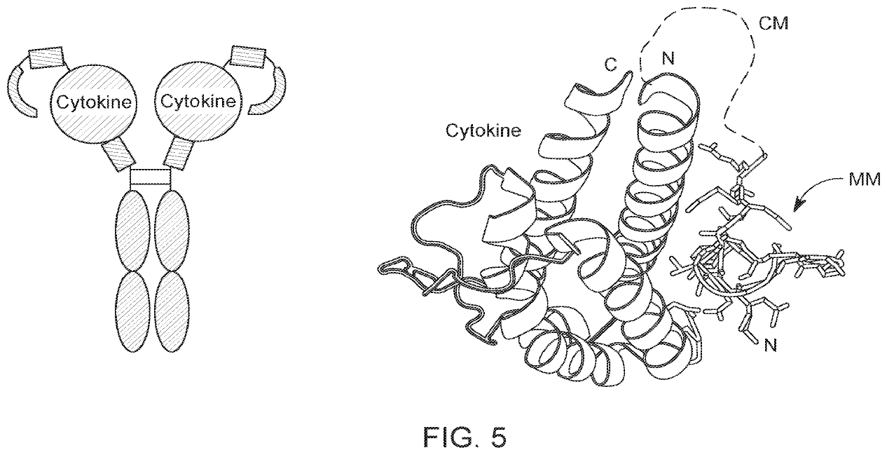

An activatable cytokine construct (ProC2970) containing human IL-15 was prepared by recombinant methods. The 1st and 2nd monomer constructs of the ProC2970 were identical, with each being a polypeptide having the amino acid sequence of SEQ ID NO: 523 and a signal sequence at its N-terminus. Each of the 1st and 2nd monomer constructs comprises, from N-terminus to C-terminus, a signal sequence from a mouse IgG kappa signal sequence (METDTLLLWVLLLWVPGSTG (SEQ ID NO: 345)), a MM (SEQ ID NO: 505), a cleavable moiety SGRSDNI (SEQ ID NO: 655), a mature cytokine protein that corresponds to human IL-15 amino acid residues 49-161 (SEQ ID NO: 347), a cleavable moiety SGRSDNI (SEQ ID NO: 655), and a dimerization domain corresponding to human IgG4 Fc, truncated at Cys226 (according to EU numbering) and including an S228P mutation (SEQ ID NO: 3) (FIG. 5). The complete monomer construct sequence for ProC2970, including the signal sequence, is shown in SEQ ID NO: 524.

A peptide mask ALTTVDGGGGSASHYFER (SEQ ID NO: 236) for IL-15 was designed from the sequence of IL-2Rβ in the crystal structure of IL-15 quaternary complex (PDB ID: 4GS7). Two peptide motifs from IL-2Rβ, KLTTVD (SEQ ID NO: 720) and ASHYFER (SEQ ID NO: 721), that make interactions with IL-15 were concatenated by a linker to develop a single concatenated peptide as cytokine masking moiety (MM). The polypeptide was prepared by transforming a host cell with a polynucleotide having the sequence of SEQ ID NO: 529, followed by cultivation of the resulting recombinant host cells. Dimerization of the resulting expressed polypeptides yielded the cytokine construct ProC2970. The polypeptide (ProC2970) was purified from the culture supernatant by Protein A and size-exclusion chromatography and was assayed to be >95% of the desired species.

### Example 2: In Vitro Characterization of Example IL-15 Cytokine Constructs

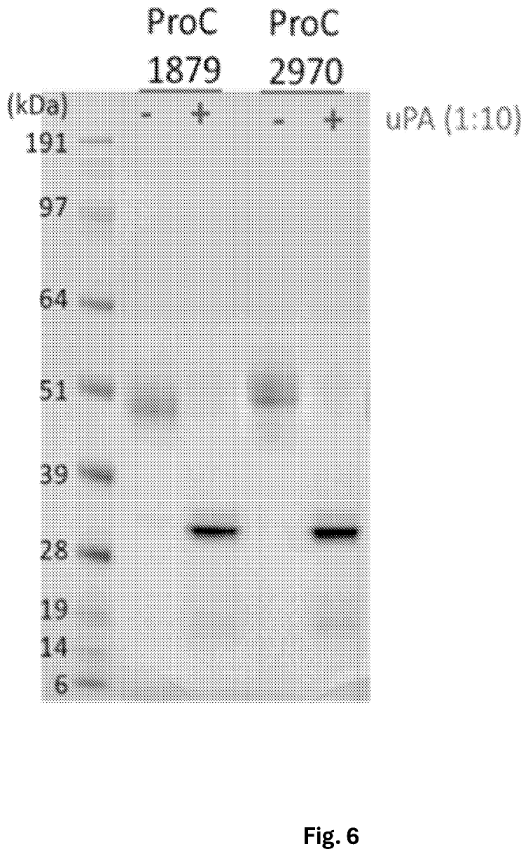

To cleave the structure-guided peptide concatenated mask, MM, and dimerization domain, the IL-15-containing ACC was treated overnight at 37° C. with recombinant human protease urokinase-type plasminogen activator (uPA). A cocktail of protease inhibitors was added to neutralize the proteases prior to testing for activity. Cleavage with uPA at the expected site in the cleavable moiety was confirmed by electrophoresis (FIG. 6). The results suggest that the uPA protease cleaves the cleavable moieties (CM) in ProC2970 and ProC1879.

The activities of ProC2970 and ProC1879 were tested in vitro using IL-2/IL-15-responsive HEK293 cells before and after cleavage with uPA. The IL-2/IL15-responsive HEK293 cells were generated by stable transfection with the human CD25 (IL-2Rα), CD122 (IL-2Rβ), and CD132 (IL-2Rγ) genes, along with the human JAK3 and STAT5 genes to obtain a fully functional IL-2/IL-15 signaling pathway. The cells also feature an STAT5-inducible SEAP (secreted embryonic alkaline phosphatase) reporter gene. To maintain transgene expression, cells were cultured in DMEM GlutaMax™ media supplemented with 10% FBS, Pen/Strep, 10 ug/ml Puromycin, and 100 μg/mL of Normocin™. The addition of IL-2 and IL-15 to these cells activates the STAT5 and subsequently induces the production of SEAP which can be readily assessed in the supernatant using QUANTI-Blue solution, a colorimetric detection for alkaline phosphatase activity.

IL-2/IL-15-responsive HEK293 cells were prepared at a concentration of 280,000 cells/mL in DMEM media supplemented with 10% FBS and 180 μL aliquots were pipetted into wells of a white flat-bottom 96-well plate (50,000 cells/well). The tested cytokines were diluted in DMEM media supplemented with 10% FBS. Duplicate of three-fold serial dilutions were prepared from which 20 μL was added to the each well. After 20-24 hours of incubation at 37° C., 20 μl of supernatant of the induced reporter cells was transferred to wells of a to flat-bottom 96-well plate. 180 μl of resuspended QUANTI-Blue solution was added per well. Following incubation of the plate at 37° C. incubator for 1-3 h, the SEAP levels were measured using a spectrophotometer at 620 nm. Dose-response curves were generated and EC50 values were obtained by sigmoidal fit non-linear regression using Graph Pad Prism software.

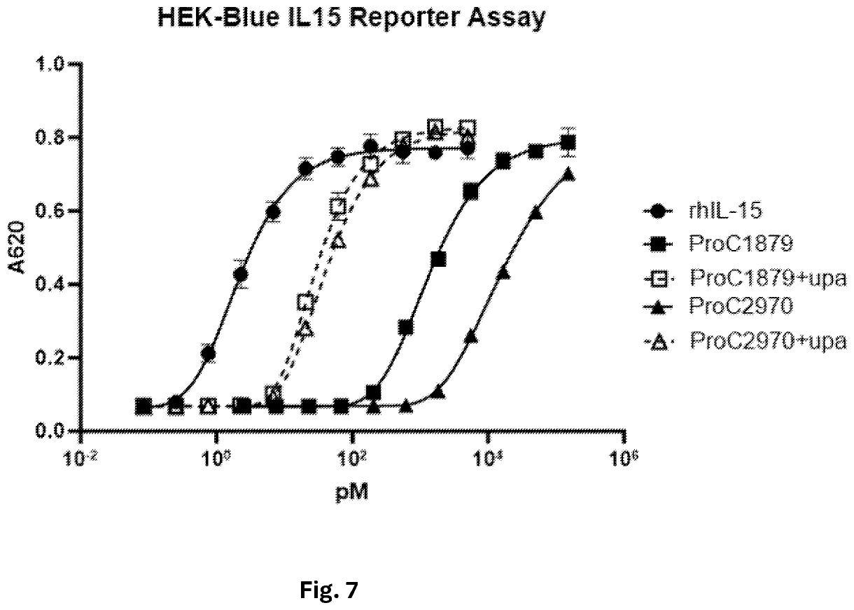

In the reporter assay, the activity of ProC2970 was reduced at least 6000× (6000-fold) as compared to PeproTech IL-15 (Recombinant human IL-15 (rhIL-15), available from PeproTech, Catalog #200-15) and 9.5× (9.5-fold) as compared to the Fc-masked IL-15 ProC1879 (SEQ ID NO: 356) (FIG. 7). This indicates that the fusion of a MM according to the present disclosure provided additional masking to IL-15 in the ACC construct. Protease activation with uPA partially restored activity of ProC2970 to a level close to but lower than the recombinant IL-15. EC50 values for rhIL-15, ProC1879, ProC2970, ProC1879+uPA, and ProC2970+uPA are provided below in Table 4.

TABLE 4
EC50: HEK-Blue Reporter Assay

| | | | | ProC1879 + | ProC2970 + |
|---|---|---|---|---|---|
| | rhIL-15 | ProC1879 | ProC2970 | uPA | uPA |
| EC50 | 2.133 | 1413 | 13817 | 30.83 | 43.71 |
| (pM) | | | | | |

### Example 3: Activity of IL-15-Containing ACCs on Human PBMC Proliferation

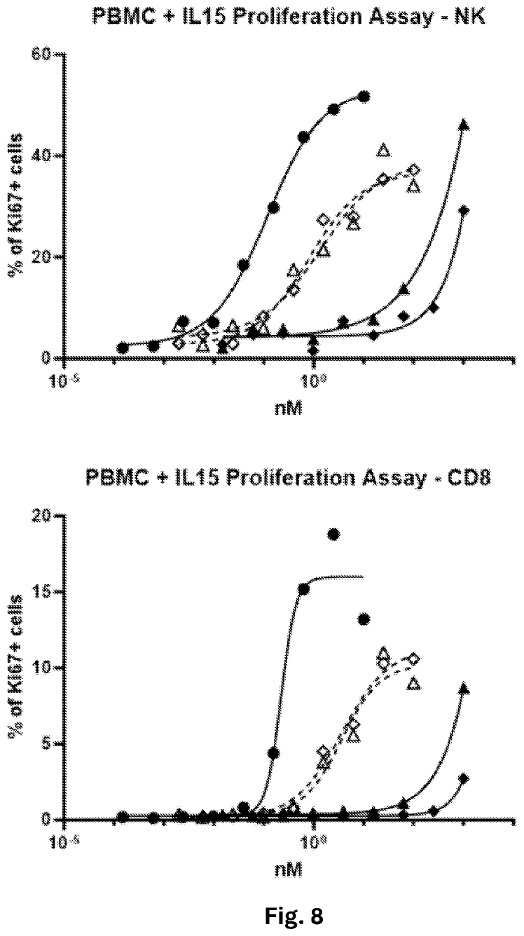

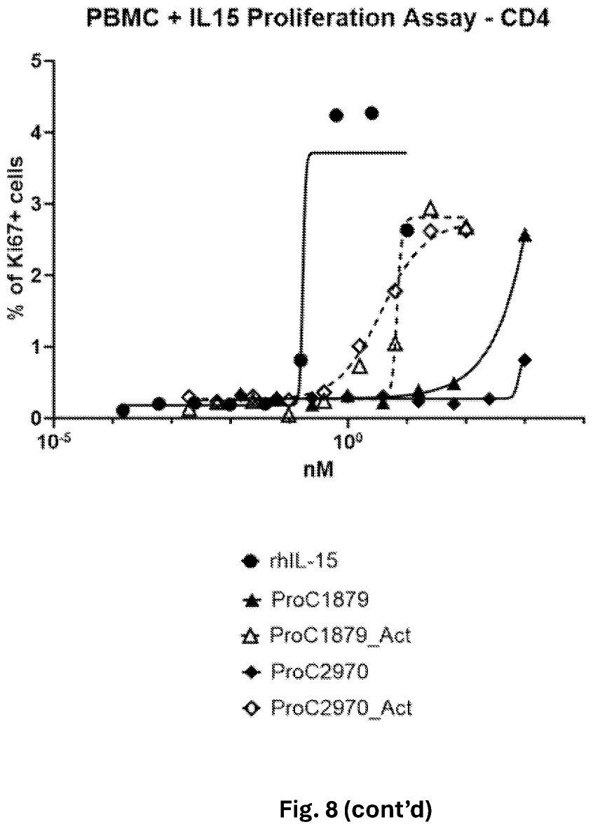

In the cell proliferation assay, human PBMCs were incubated with recombinant IL-15 or IL-15-ACCs (with or without prior-protease activation) for 3 days. Following incubation, PBMCs were stained with fixable viability dye eFlur™780, anti-CD3-FITC (UCHTI), anti-CD4-BV608 (RPA-T4), anti-CD8-BV480 (RPA-T8), anti-CD56-BV421 (HCD56), and anti-Ki67-APC (Ki67) antibodies. Various cell populations including CD3−, CD56+NK cells, CD3+, CD8+ T cells and CD3+, CD4+ T cells were analyzed, and proliferation of the various cell populations were determined based on percentage Ki67 expression, as shown in FIG. 8. Protease-treated IL-15-ACC s show stronger proliferative activity than the corresponding intact IL-15-containing ACCs. Table 5 shows the EC50 of various IL-15-containing ACCs in the PBMC.

TABLE 5
EC50: Human PBMC Proliferation Ki67

| EC50 (nM) | NK Cells | CD8 | CD4 |
|---|---|---|---|
| rhIL-15 | 0.1167 | 0.2225 | 0.1701 |
| ProC1879 + uPA | 1.352 | 3.584 | 6.779 |
| ProC2970 + uPA | 0.8067 | 3.644 | 3.673 |
| ProC1879 | 71560 | 721308 | 28697 |
| ProC2970 | 3392706 | 41289 | 780.3 |

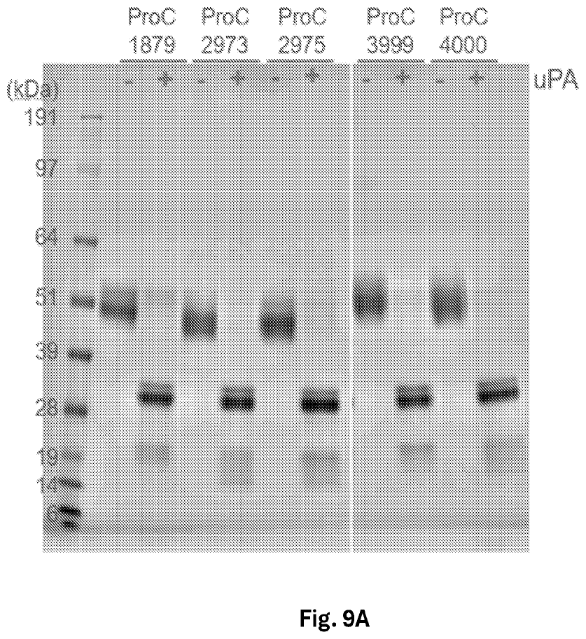

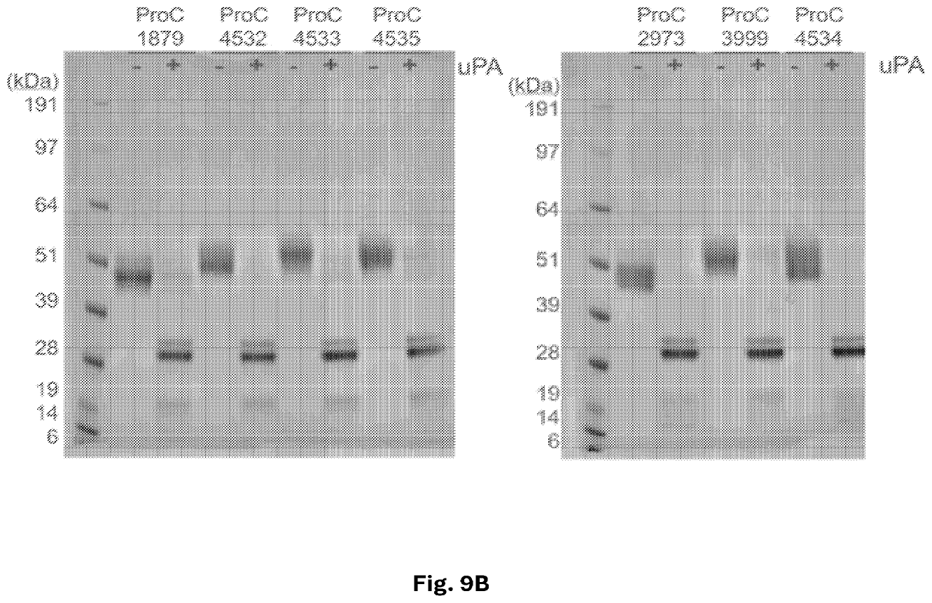

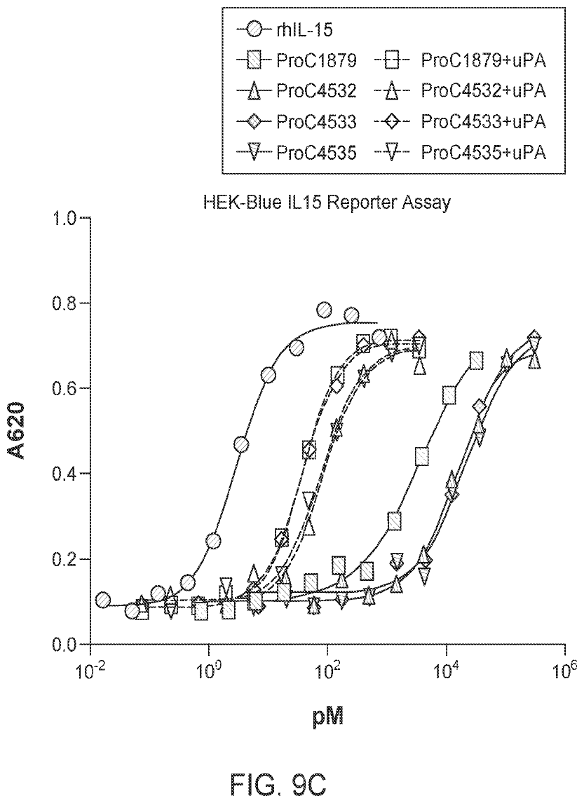

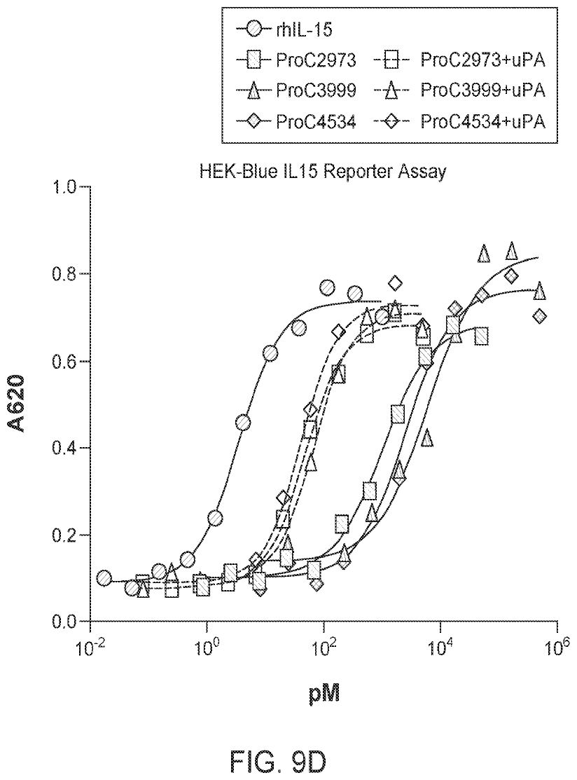

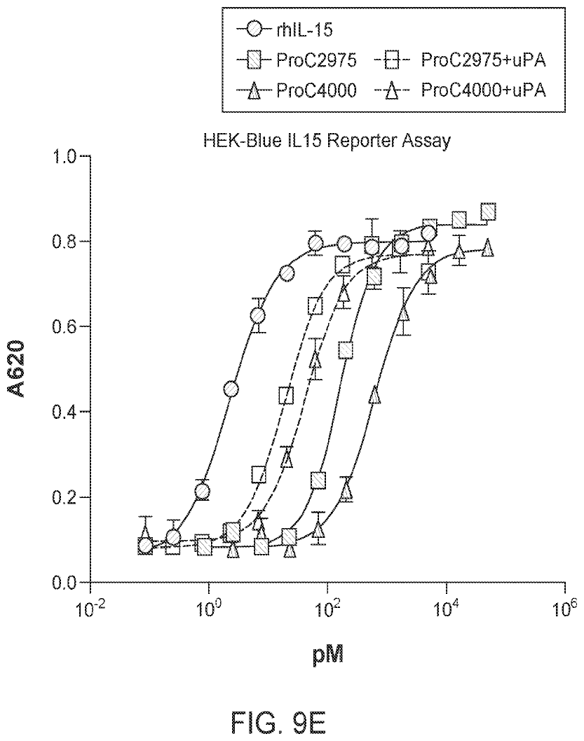

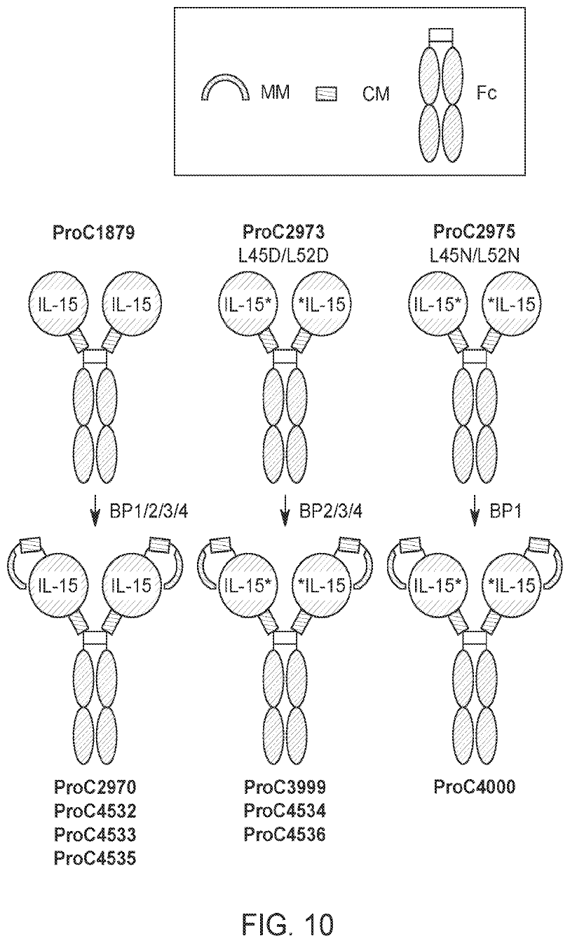

Example 4: In vitro characterization of example IL-15-containing ACCs IL-15 WT ACC and IL-15 mutein ACCs were treated overnight at 37° C. with recombinant uPA. A cocktail of protease inhibitors was added to neutralize the proteases prior to testing for activity. Table 6 shows mask sequences for IL-15. ACCs are shown schematically in FIG. 10. Activation of ACCs proceeded by incubating the constructs overnight at 37° C. using a ratio of ACC to uPA of 1 to 5. Cleavage with uPA at the expected site in the cleavable moiety was confirmed by electrophoresis (FIGS. 9A and 9B). HEK293 reporter assay characterized the activities of intact and protease-treated IL-15-containing ACCs (FIGS. 9C-9E). Table 7 shows the average EC50 values of the IL-15-containing ACCs from multiple experiments. The results show that structure-based peptide masks provide activity attenuation to both WT and mutein IL-15-containing ACCs.

TABLE 6

| | | IL15 | IL-15 | IL-15 |
|---|---|---|---|---|
| | Beta Peptide | WT | L45D/L52D | L45N/L52N |
| | Mask | ProC1879 | ProC2973 | ProC2975 |
| BP1 | ----ALTTVDGGG | ProC2970 | | ProC4000 |
| | GSASHYFER--- | | | |
| | (SEQ ID NO: 236) | | | |
| BP2 | ----KLTTVRGGG | ProC4532 | ProC3999 | |
| | GSASHYFER--- | | | |
| | (SEQ ID NO: 734) | | | |
| BP3 | (SQ)KLTTVDGGG | ProC4533 | ProC4534 | |
| | GSASHYFER(HLE) | | | |
| | (SEQ ID NO: 730) | | | |
| BP4 | (SQ)KLTTVRGGG | ProC4535 | ProC4536 | |
| | GSASHYFER(HLE) | | | |
| | (SEQ ID NO: 731) | | | |

A linker sequence is shown underlined, N-terminal and C-terminal extension shown in parenthesis, and variable amino acids are shown in bold.

TABLE 7
EC50: HEK-Blue Reporter Assay

| Cytokine | | | | | Activity fold-change |
|---|---|---|---|---|---|
| Molecule | | BP | Intact EC50 | Activated | intact ACC/ activated |
| Tested | IL-15 | mask | (pM) | EC50 (pM) | treated ACC |
| rhIL-15 | | | 2.93 | | |
| ProC1879 | WT | — | 4070.3 | 60.3 | 67 |
| ProC4532 | WT | BP2 | 33357.8 | 166.3 | 201 |
| ProC4533 | WT | BP3 | 22121.0 | 82.6 | 268 |
| ProC4535 | WT | BP4 | 34756.5 | 197.5 | 176 |
| ProC2973 | L45D/ | — | 576.6 | 45.5 | 13 |
| | L52D | | | | |
| ProC3999 | L45D/ | BP2 | 3734.0 | 78.2 | 48 |
| | L52D | | | | |
| ProC4534 | L45D/ | BP3 | 2257.6 | 39.5 | 57 |
| | L52D | | | | |
| ProC2975 | L45N/ | — | 166.3 | 18.6 | 9 |
| | L52N | | | | |
| ProC4000 | L45N/ | BP1 | 628.4 | 41.7 | 15 |
| | L52N | | | | |

TABLE 8
Example Sequences

| SEQ | | |
|---|---|---|
| ID NO. | NAME | SEQUENCE |
| 2 | Linker | GGGS |
| 3 | Human IgG4 Fc | CPPCPAPEFLGGPSVFLFPPKPKDTLMISRTPEVTCVV |
| | Region with S228P | VDVSQEDPEVQFNWYVDGVEVHNAKTKPREEQFNSTY |
| | mutation, truncated | RVVSVLTVLHQDWLNGKEYKCKVSNKGLPSSIEKTISK |
| | to Cys226 | AKGQPREPQVYTLPPSQEEMTKNQVSLTCLVKGFYPS |
| | | DIAVEWESNGQPENNYKTTPPVLDSDGSFFLYSRLTVD |
| | | KSRWQQGNVFSCSVMHEALHNHYTQKSLSLS |
| 4 | Human IgG4 Fc | ESKYGPPCPPCPAPEFLGGPSVFLFPPKPKDTLMISRT |
| | Region with S228P | PEVTCVVVDVSQEDPEVQFNWYVDGVEVHNAKTKPREE |
| | mutation and full | QFNSTYRVVSVLTVLHQDWLNGKEYKCKVSNKGLPSS |
| | hinge region | IEKTISKAKGQPREPQVYTLPPSQEEMTKNQVSLTCLV |
| | | KGFYPSDIAVEWESNGQPENNYKTTPPVLDSDGSFFLY |
| | | SRLTVDKSRWQQGNVFSCSVMHEALHNHYTQKSLSLS |
| 119 | Human IL-2 | MYRMQLLSCIALSLALVTNSAPTSSSTKKTQLQLEHLL |
| | | LDLQMILNGINNYKNPKLTRMLTFKFYMPKKATELKH |
| | | LQCLEEELKPLEEVLNLAQSKNFHLRPRDLISNINVIVL |
| | | ELKGSETTFMCEYADETATIVEFLNRWITFCQSIIS |
| 120 | Mouse IL-2 | MYSMQLASCVTLTLVLLVNSAPTSSSTSSSTAEAQQQQ |
| | | QQQQQQQQHLEQLLMDLQELLSRMENYRNLKLPRML |
| | | TFKFYLPKQATELKDLQCLEDELGPLRHVLDLTQSKSF |
| | | QLEDAENFISNIRVTVVKLKGSDNTFECQFDDESATVV |
| | | DFLRRWIAFCQSIISTSPQ |
| 121 | Human IL-4 | MGLTSQLLPPLFFLLACAGNFVHGHKCDITLQEIIKTLN |
| | | SLTEQKTLCTELTVTDIFAASKNTTEKETFCRAATVLR |
| | | QFYSHHEKDTRCLGATAQQFHRHKQLIRFLKRLDRNL |
| | | WGLAGLNSCPVKEANQSTLENFLERLKTIMREKYSKC |
| | | SS |
| 122 | Mouse IL-4 | MGLNPQLVVILLFFLECTRSHIHGCDKNHLREIIGILNE |
| | | VTGEGTPCTEMDVPNVLTATKNTTESELVCRASKVLRI |
| | | FYLKHGKTPCLKKNSSVLMELQRLFRAFRCLDSSISCT |
| | | MNESKSTSLKDFLESLKSIMQMDYS |
| 123 | Human IL-7 | MFHVSFRYIFGLPPLILVLLPVASSDCDIEGKDGKQYES |
| | | VLMVSIDQLLDSMKEIGSNCLNNEFNFFKRHICDANKE |
| | | GMFLFRAARKLRQFLKMNSTGDFDLHLLKVSEGTTILL |
| | | NCTGQVKGRKPAALGEAQPTKSLEENKSLKEQKKLND |
| | | LCFLKRLLQEIKTCWNKILMGTKEH |
| 124 | Mouse IL-7 | MFHVSFRYIFGIPPLILVLLPVTSSECHIKDKEGKAYESV |
| | | LMISIDELDKMTGTDSNCPNNEPNFFRKHVCDDTKEAA |
| | | FLNRAARKLKQFLKMNISEEFNVHLLTVSQGTQTLVN |
| | | CTSKEEKNVKEQKKNDACFLKRLLREIKTCWNKILKG |
| | | SI |
| 125 | Human IL-9 | MLLAMVLTSALLLCSVAGQGCPTLAGILDINFLINKMQ |
| | | EDPASKCHCSANVTSCLCLGIPSDNCTRPCFSERLSQMT |
| | | NTTMQTRYPLIFSRVKKSVEVLKNNKCPYFSCEQPCNQ |
| | | TTAGNALTFLKSLLEIFQKEKMRGMRGKI |
| 126 | Mouse IL-9 | MLVTYILASVLLFSSVLGQRCSTTWGIRDTNYLIENLK |
| | | DDPPSKCSCSGNVTSCLCLSVPTDDCTTPCYREGLLQL |
| | | TNATQKSRLLPVFHRVKRIVEVLKNITCPSFSCEKPCN |
| | | QTMAGNTLSFLKSLLGTFQKTEMQRQKSRP |
| 129 | Human IL-15 | MRISKPHLRSISIQCYLCLLLNSHFLTEAGIHVFILGCFS |
| | | AGLPKTEANWVNVISDLKKIEDLIQSMHIDATLYTESD |
| | | VHPSCKVTAMKCFLLELQVISLESGDASIHDTVENLIIL |
| | | ANNSLSSNGNVTESGCKECEELEEKNIKEFLQSFVHIVQ |
| | | MFINTS |
| 130 | Mouse IL-15 | MKILKPYMRNTSISCYLCFLLNSHFLTEAGIHVFILGCV |
| | | SVGLPKTEANWIDVRYDLEKIESLIQSIHIDTTLYTDSDF |
| | | HPSCKVTAMNCFLLELQVILHEYSNMTLNETVRNVLY |
| | | LANSTLSSNKNVAESGCKECEELEEKTFTEFLQSFIRIV |
| | | QMFINTS |
| 210 | Linker | GSSGGSGGSGG |
| 211 | Linker | GGGSGGGS |
| 212 | Linker | GGGSGGGSGGGS |
| 213 | Linker | GGGGSGGGGSGGGGS |
| 214 | Linker | GGGGSGGGGSGGGGSGGGGSGGGGS |
| 215 | Linker | GGGGSGGGGS |
| 216 | Linker | (GGGGS)n |
| 217 | Linker | GGGGSGS |
| 218 | Linker | GGGGSGGGGSGGGGSGS |
| 219 | Linker | GGSLDPKGGGGS |
| 220 | Linker | PKSCDKTHTCPPCPAPELLG |
| 221 | Linker | SKYGPPCPPCPAPEFLG |
| 222 | Linker | GKSSGSGSESKS |
| 223 | Linker | GSTSGSGKSSEGKG |
| 224 | Linker | GSTSGSGKSSEGSGSTKG |
| 225 | Linker | GSTSGSGKPGSGEGSTKG |
| 226 | Linker | GSTSGSGKPGSSEGST |
| 227 | Linker | (GSGGS)n |
| 228 | Linker | (GGGS)n |
| 229 | Linker | GGSG |
| 230 | Linker | GGSGG |
| 231 | Linker | GSGSG |
| 232 | Linker | GSGGG |
| 233 | Linker | GGGSG |
| 234 | Linker | GSSSG |
| 235 | Linker | GGGGSGGGGSGGGGSGGGGS |
| 245 | Linker | GPQGTAGQ |
| 250 | Linker | YGAGLGW |
| 315 | human IgG Fc with | CPPCPAPEFEGGPSVFLFPPKPKDTLMISRTPEVTCVV |
| | a knob mutation | VDVSQEDPEVQFNWYVDGVEVHNAKTKPREEQFNSTY |
| | | RVVSVLTVLHQDWLNGKEYKCKVSNKGLPSSIEKTISK |
| | | AKGQPREPQVYTLPPCQEEMTKNQVSLWCLVKGFYPS |
| | | DIAVEWESNGQPENNYKTTPPVLDSDGSFFLYSRLTVD |
| | | KSRWQEGNVFSCSVMHEALHNHYTQKSLSLSLG |
| 316 | human IgG Fc with | CPPCPAPEFEGGPSVFLFPPKPKDTLMISRTPEVTCVVV |
| | a hole mutation | DVSQEDPEVQFNWYVDGVEVHNAKTKPREEQFNSTY |
| | | RVVSVLTVLHQDWLNGKEYKCKVSNKGLPSSIEKTISK |
| | | AKGQPREPQVCTLPPSQEEMTKNQVSLSCAVKGFYPS |
| | | DIAVEWESNGQPENNYKTTPPVLDSDGSFFLVSRLTVD |
| | | KSRWQEGNVFSCSVMHEALHNRFTQKSLSLSLG |
| 318 | Linker | GSSGGS |
| 319 | Linker | ESKY |
| 321 | Linker | SGGG |
| 216 | Linker | GGGGS |
| 322 | Linker | PGGGS |
| 323 | Linker | PGPPS |
| 324 | Linker | GGPPS |
| 325 | Linker | GCPPC |
| 326 | Linker | PCPPC |
| 327 | Linker | GPGGS |
| 335 | Linker | SGGGG |
| 343 | Signal sequence | MRAWIFFLLCLAGRALA |
| 344 | Signal sequence | MALTFALLVALLVLSCKSSCSVG |
| 345 | Signal sequence | METDTLLLWVLLLWVPGSTG |
| 347 | Human IL-15 | NWVNVISDLKKIEDLIQSMHIDATLYTESDVHPSCKVT |
| | (amino acids 49- | AMKCFLLELQVISLESGDASIHDTVENLIILANNSLSSN |
| | 161) | GNVTESGCKECEELEEKNIKEFLQSFVHIVQMFINT |
| 348 | Human IL-15 | NWVNVISDLKKIEDLIQSMHIDATLYTESDVHPSCKVT |
| | (amino acids 49- | AMKCFLLELQVISLESGDASIHDTVENLIILANNSLSSN |
| | 162) | GNVTESGCKECEELEEKNIKEFLQSFVHIVQMFINTS |
| 356 | ProC1879 | NWVNVISDLKKIEDLIQSMHIDATLYTESDVHPSCKVT |
| | | AMKCFLLELQVISLESGDASIHDTVENLIILANNSLSSN |
| | | GNVTESGCKECEELEEKNIKEFLQSFVHIVQMFINTS |
| | | GGLSGRSNICPPCPAPEFLGGPSVFLFPPKPKDTLMISRT |
| | | PEVTCVVVDVSQEDPEVQFNWYVDGVEVHNAKTKPR |
| | | EEQFNSTYRVVSVLTVLHQDWLNGKEYKCKVSNKGL |
| | | PSSIEKTISKAKGQPREPQVYTLPPSQEEMTKNQVSLTC |
| | | LVKGFYPSDIAVEWESNGQPENNYKTTPPVLDSDGSFF |
| | | LYSRLTVDKSRWQQGNVFSCSVMHEALHNHYTQKSL |
| | | SLS |
| 358 | IL-15 binding | AVNGTSQFTCFYNSRANISCVWSQDGALQDTSCQVHA |
| | protein | WPDRRRWNQTCELLPVSQASWACNLILGAPDSQKLTT |
| | | VDIVTLRVLCREGVRWRVMAIQDFKPFENLRLMAPISL |
| | | QVVHVETHRCNISWEISQASHYFERHLEFEARTLSPGH |
| | | TWEEAPLLTLKQKQEWICLETLTPDTQYEFQVRVKPLQ |
| | | GEFTTWSPWSQPLAFRTKPAALGKDT |
| 359 | IL-15 binding | ITCPPPMSVEHADIWVKSYSLYSRERYICNSGFKRKAG |
| | protein | TSSLTECVLNKATNVAHWTTPSLKCIRDPALVHQRPAP |
| | | P |
| 360 | IL-15 binding | ITCPPPMSVEHADIWVKSYSLYSRERYICNSGFKRKAG |
| | protein | TSSLTECVLNKATNVAHWTTPSLKCIRDP |
| 361 | IL-15 binding | ITCPPPMSVEHADIWVKSYSLYSRERYICNSGFKRKAG |
| | protein | TSSLTECVLNKATNVAHWTTPSLKCIR |
| 362 | IL-15 binding | ITCPPPMSVEHADIWVKSYSLYSRERYICNSGFKRKAG |
| | protein | TSSLTECVLNKATNVAHWTTPSLKCIRDPALVHQRPAP |
| | | PSTVTTAGVTPQPESLSPSGKEPAASSPSSNNTAATTAA |
| | | IVPGSQLMPSKSPSTGTTEISSHESSHGTPSQTTAKNWE |
| | | LTASASHQPPGVYPQGHSDTT |
| 363 | IL-15 binding | ITCPPPMSVEHADIWVKSYSLYSRERYICNSGFKRKAG |
| | protein | TSSLTECVLNKATNVAHWTTPSLKCIRDPALVHQRPAP |
| | | PSTVTTAGVTPQPESLSPSGKEPAAS |
| 364 | IL-15 binding | MAPRRARGCRTLGLPALLLLLLLRPPATRGITCPPPMS |
| | protein | VEHADIWVKSYSLYSRERYICNSGFKRKAGTSSLTECV |
| | | LNKATNVAHWTTPSLKCIRDPALVHQRPAPPSTVTTAG |
| | | VTPQPESLSPSGKEPAASSPSSNNTAATTAAIVPGSQLM |
| | | PSKSPSTGTTEISSHESSHGTPSQTTAKNWELTASASHQ |
| | | PPGVYPQGHSDTTVAISTSTVLLCGLSAVSLLACYLKS |
| | | RQTPPLASVEMEAMEALPVTWGTSSRDEDLENCSHHL |
| 365 | IL-2 or IL-15 | AVNGTSQFTCFYNSRANISCVWSQDGALQDTSCQVHA |
| | binding protein | WPDRRRWNQTCELLPVSQASWACNLILGAPESQKLTT |
| | | VDIVTLRVLCREGVRWRVMAIQDFKPFENLRLMAPISL |
| | | QVVHVETHRCNISWEISQASHYFERHLEFEARTLSPGH |
| | | TWEEAPLLTLKQKQEWICLETLTPDTQYEFQVRVKPLQ |
| | | GEFTTWSPWSQPLAFRTKPAALGKDT |
| 366 | IL-2 or IL-15 | AVNGTSQFTCFYNSRANISCVWSQDGALQDTSCQVHA |
| | binding protein | WPDRRRWNQTCELLPVSQASWACNLILGAPDHQKLTT |
| | | VDIVTLRVLCREGVRWRVMAIQDFKPFENLRLMAPISL |
| | | QVVHVETHRCNISWEISQASHYFERHLEFEARTLSPGH |
| | | TWEEAPLLTLKQKQEWICLETLTPDTQYEFQVRVKPLQ |
| | | GEFTTWSPWSQPLAFRTKPAALGKDT |
| 367 | IL-2 or IL-15 | AVNGTSQFTCFYNSRANISCVWSQDGALQDTSCQVHA |
| | binding protein | WPDRRRWNQTCELLPVSQASWACNLILGAPDSQKLTT |
| | | QDIVTLRVLCREGVRWRVMAIQDFKPFENLRLMAPISL |
| | | QVVHVETHRCNISWEISQASHYFERHLEFEARTLSPGH |
| | | TWEEAPLLTLKQKQEWICLETLTPDTQYEFQVRVKPLQ |
| | | GEFTTWSPWSQPLAFRTKPAALGKDT |
| 368 | IL-2 or IL-15 | AVNGTSQFTCFYNSRANISCVWSQDGALQDTSCQVHA |
| | binding protein | WPDRRRWNQTCELLPVSQASWACNLILGAPDSQKLTT |
| | | FDIVTLRVLCREGVRWRVMAIQDFKPFENLRLMAPISL |
| | | QVVHVETHRCNISWEISQASHYFERHLEFEARTLSPGH |
| | | TWEEAPLLTLKQKQEWICLETLTPDTQYEFQVRVKPLQ |
| | | GEFTTWSPWSQPLAFRTKPAALGKDT |
| 369 | IL-2 or IL-15 | AVNGTSQFTCFYNSRANISCVWSQDGALQDTSCQVHA |
| | binding protein | WPDRRRWNQTCELLPVSQASWACNLILGAPDSQKLTT |
| | | VDIVTLRVLCREGVRWRVMAIQDFKPFENLRLMAPISL |
| | | QVVHVETHRCNISWEISQASHYFQRHLEFEARTLSPGH |
| | | TWEEAPLLTLKQKQEWICLETLTPDTQYEFQVRVKPLQ |
| | | GEFTTWSPWSQPLAFRTKPAALGKDT |
| 370 | IL-2 or IL-15 | AVNGTSQFTCFYNSRANISCVWSQDGALQDTSCQVHA |
| | binding protein | WPDRRRWNQTCELLPVSQASWACNLILGAPDSQKLTT |
| | | VDIVTLRVLCREGVRWRVMAIQDFKPFENLRLMAPISL |
| | | QVVHVETHRCNISWEISQASHYFQRRLEFEARTLSPGH |
| | | TWEEAPLLTLKQKQEWICLETLTPDTQYEFQVRVKPLQ |
| | | GEFTTWSPWSQPLAFRTKPAALGKDT |
| 371 | IL-2 or IL-15 | AVNGTSQFTCFYNSYANISCVWSQDGALQDTSCQVHA |
| | binding protein | WPDRRRWNQTCELLPVSQASWACNLILGAPDSQKLTT |
| | | VDIVTLRVLCREGVRWRVMAIQDFKPFENLRLMAPISL |
| | | QVVHVETHRCNISWEISQASHYFERHLEFEARTLSPGH |
| | | TWEEAPLLTLKQKQEWICLETLTPDTQYEFQVRVKPLQ |
| | | GEFTTWSPWSQPLAFRTKPAALGKDT |
| 372 | IL-2 or IL-15 | LNTTILTPNGNEDTTADFFLTTMPTDSLSVSTLPLPEV |
| | binding protein | QCFVFNVEYMNCTWNSSSEPQPTNLTLHYWYKNSDND |
| | | KVQKCSHYLFSEEITSGCQLQKKEIHLYQTFVVQLQDP |
| | | REPRRQATQMLKLQNLVIPWAPENLTLHKLSESQLELN |
| | | WNNRFLNHCLEHLVQYRTDWDHSWTEQSVDYRHKFS |
| | | LPSVDGQKRYTFRVRSRFNPLCGSAQHWSEWSHPIHW |
| | | GSNTSKENPFLFALEA |
| 373 | IL-2 or IL-15 | CPDLVCYTDYLQTVICILEMWNLHPSTLTLTWQDQYE |
| | binding protein | ELKDEATSCSLHRSAHNATHATYTCHMDVFHFMADDI |
| | | FSVNITDQSGNYSQECGSFLLAESIKPAPPFNVTVTFSG |
| | | QYNISWRSDYEDPAFYMLKGKLQYELQYRNRGDPWA |
| | | VSPRRKLISVDSRSVSLLPLEFRKDSSYELQVRAGPMPG |
| | | SSYQGTWSEWSDPVIFQTQSEELKE |
| 374 | IL-15 binding | ITCPPPMSVEHADIWVKSYSLYSRERYICNSGFKRKAG |
| | protein | TSSLTECVLNKATNVAHWTTPSLKCIRDPALVHQRPAP |
| | | PS |
| 375 | Spacer | QGQSGS |
| 376 | Spacer | GQSGS |
| 377 | Spacer | QSGS |
| 378 | Spacer | QGQSGQG |
| 379 | Spacer | GQSGQG |
| 380 | Spacer | QSGQG |
| 381 | Spacer | SGQG |
| 382 | Spacer | QGQSGQ |
| 383 | Spacer | GQSGQ |
| 384 | Spacer | QSGQ |
| 385 | Spacer | QGQSG |
| 386 | Spacer | QGQS |
| 387 | N-terminal | EPKSCDKTHT |
| | sequence in Fc | |
| 388 | N-terminal | ELKTPLGDTTHT |
| | sequence in Fc | |
| 389 | N-terminal | ESKYGPP |
| | sequence in Fc | |
| 391 | L10R1P1_1490DN | TNTRFSVDEVTGGGGSSVASRSNKGGGISSGLLSGRSD |
| | P_IL- | NPGGSCTHFPGNLPNMLRDLRDAFSRVKTFFQMKDQL |
| | 10_1490DNP_IgG4 | DNLLLKESLLEDFKGYLGCQALSEMIQFYLEEVMPQAE |
| | (C226) | NQDPDIKAHVNSLGENLKTLRLRLRRCHRFLPCENKSK |
| | | AVEQVKNAFNKLQEKGIYKAMSEFDIFINYIEAYMTM |
| | | KIRNGGISSGLLSGRSDNPCPPCPAPEFLGGPSVFLFP |
| | | PKPKDTLMISRTPEVTCVVVDVSQEDPEVQFNWYVDGVE |
| | | VHNAKTKPREEQFNSTYRVVSVLTVLHQDWLNGKEY |
| | | KCKVSNKGLPSSIEKTISKAKGQPREPQVYTLPPSQEEM |
| | | TKNQVSLTCLVKGFYPSDIAVEWESNGQPENNYKTTPP |
| | | VLDSDGSFFLYSRLTVDKSRWQQGNVFSCSVMHEALH |
| | | NHYTQKSLSLS |
| 392 | L18RBP1_1490DN | VDEVYDYHQGGGGSLLLGSTGGGGSISSGLLSGRSDNP |
| | P_IL- | GGGGSYFIAEDDENLESDYFGKLESKLSVIRNLNDQVL |
| | 18_1490DNP_IgG4 | FIDQGNRPLFEDMTDSDCRDNAPRTIFIISMYKDSQPRG |
| | (C226) | MAVTISVKCEKISTLSCENKIISFKEMNPPDNIKDTKSDI |
| | | IFFQRSVPGHDNKMQFESSSYEGYFLACEKERDLFKLIL |
| | | KKEDELGDRSIMFTVQNEDGGISSGLLSGRSDNPCPPCP |
| | | APEFLGGPSVFLFPPKPKDTLMISRTPEVTCVVVDVSQE |
| | | DPEVQFNWYVDGVEVHNAKTKPREEQFNSTYRVVSV |
| | | LTVLHQDWLNGKEYKCKVSNKGLPSSIEKTISKAKGQP |
| | | REPQVYTLPPSQEEMTKNQVSLTCLVKGFYPSDIAVEW |
| | | ESNGQPENNYKTTPPVLDSDGSFFLYSRLTVDKSRWQ |
| | | QGNVFSCSVMHEALHNHYTQKSLSLS |
| 393 | IFNR1P1_1490DN | EEFAVLRDGKGGGGSGVLNVWGVGGISSGLLSGRSDN |
| | P_IFNy_1490DNP | PGGGSQDPYVKEAENLKKYFNAGHSDVADNGTLFLGI |
| | IgG4(C226) | LKNWKEESDRKIMQSQIVSFYFKLFKNFKDDQSIQKSV |
| | | ETIKEDMNVKFFNSNKKKRDDFEKLTNYSVTDLNVQR |
| | | KAIHELIQVMAELSPAAKTGKRKRSQMLFRGRRASQG |
| | | GISSGLLSGRSDNPCPPCPAPEFLGGPSVFLFPPKPKD |
| | | TLMISRTPEVTCVVVDVSQEDPEVQFNWYVDGVEVHNA |
| | | KTKPREEQFNSTYRVVSVLTVLHQDWLNGKEYKCKVS |
| | | NKGLPSSIEKTISKAKGQPREPQVYTLPPSQEEMTKNQ |
| | | VSLTCLVKGFYPSDIAVEWESNGQPENNYKTTPPVLDS |
| | | DGSFFLYSRLTVDKSRWQQGNVFSCSVMHEALHNHYT |
| | | QKSLSLS |
| 394 | ProC4532 | KLTTVRGGGGSASHYFERGGLSGRSNIGGNWVNVISD |
| | BP2_(GG)1205(G | LKKIEDLIQSMHIDATLYTESDVHPSCKVTAMKCFLLE |
| | G)_IL- | LQVISLESGDASIHDTVENLIILANNSLSSNGNVTESGC |
| | 15_(GG)1205_IgG | KECEELEEKNIKEFLQSFVHIVQMFINTSGGLSGRSNICP |
| | 4(C226) Fc Homo | PCPAPEFLGGPSVFLFPPKPKDTLMISRTPEVTCVVVDV |
| | | SQEDPEVQFNWYVDGVEVHNAKTKPREEQFNSTYRV |
| | | VSVLTVLHQDWLNGKEYKCKVSNKGLPSSIEKTISKAK |
| | | GQPREPQVYTLPPSQEEMTKNQVSLTCLVKGFYPSDIA |
| | | VEWESNGQPENNYKTTPPVLDSDGSFFLYSRLTVDKSR |
| | | WQQGNVFSCSVMHEALHNHYTQKSLSLS |
| 395 | ProC4533 | SQKLTTVDGGGGSASHYFERHLEGGLSGRSNIGGNWV |
| | BP2_(GG)1205(G | NVISDLKKIEDLIQSMHIDATLYTESDVHPSCKVTAMK |
| | G)_IL- | CFLLELQVISLESGDASIHDTVENLIILANNSLSSNGNVT |
| | 15_(GG)1205_IgG | ESGCKECEELEEKNIKEFLQSFVHIVQMFINTSGGLSGR |
| | 4(C226) Fc Homo | SNICPPCPAPEFLGGPSVFLFPPKPKDTLMISRTPEVTCV |
| | | VVDVSQEDPEVQFNWYVDGVEVHNAKTKPREEQFNS |
| | | TYRVVSVLTVLHQDWLNGKEYKCKVSNKGLPSSIEKTI |
| | | SKAKGQPREPQVYTLPPSQEEMTKNQVSLTCLVKGFY |
| | | PSDIAVEWESNGQPENNYKTTPPVLDSDGSFFLYSRLT |
| | | VDKSRWQQGNVFSCSVMHEALHNHYTQKSLSLS |
| 396 | ProC4534 | SQKLTTVDGGGGSASHYFERHLEGGLSGRSNIGGNWV |
| | BP3_(GG)1205(G | NVISDLKKIEDLIQSMHIDATLYTESDVHPSCKVTAMK |
| | G) IL- | CFLDELQVISDESGDASIHDTVENLIILANNSLSSNGNV |
| | 15(L45D/L52D)_( | TESGCKECEELEEKNIKEFLQSFVHIVQMFINTSGGLSG |
| | GG)1205_IgG4(C2 | RSNICPPCPAPEFLGGPSVFLFPPKPKDTLMISRTPEVTC |
| | 26) Fc Homo | VVVDVSQEDPEVQFNWYVDGVEVHNAKTKPREEQFN |
| | | STYRVVSVLTVLHQDWLNGKEYKCKVSNKGLPSSIEK |
| | | TISKAKGQPREPQVYTLPPSQEEMTKNQVSLTCLVKGF |
| | | YPSDIAVEWESNGQPENNYKTTPPVLDSDGSFFLYSRL |
| | | TVDKSRWQQGNVFSCSVMHEALHNHYTQKSLSLS |
| 397 | ProC4535 | SQKLTTVRGGGGSASHYFERHLEGGLSGRSNIGGNWV |
| | BP4_(GG)1205(G | NVISDLKKIEDLIQSMHIDATLYTESDVHPSCKVTAMK |
| | G)_IL- | CFLLELQVISLESGDASIHDTVENLIILANNSLSSNGNVT |
| | 15_(GG)1205_IgG | ESGCKECEELEEKNIKEFLQSFVHIVQMFINTSGGLSGR |
| | 4(C226) Fc Homo | SNICPPCPAPEFLGGPSVFLFPPKPKDTLMISRTPEVTCV |
| | | VVDVSQEDPEVQFNWYVDGVEVHNAKTKPREEQFNS |
| | | TYRVVSVLTVLHQDWLNGKEYKCKVSNKGLPSSIEKTI |
| | | SKAKGQPREPQVYTLPPSQEEMTKNQVSLTCLVKGFY |
| | | PSDIAVEWESNGQPENNYKTTPPVLDSDGSFFLYSRLT |
| | | VDKSRWQQGNVFSCSVMHEALHNHYTQKSLSLS |
| 398 | ProC4536 | SQKLTTVRGGGGSASHYFERHLEGGLSGRSNIGGNWV |
| | BP4_(GG)1205(G | NVISDLKKIEDLIQSMHIDATLYTESDVHPSCKVTAMK |
| | G)_IL- | CFLDELQVISDESGDASIHDTVENLIILANNSLSSNGNV |
| | 15(L45D/L52D)_ | TESGCKECEELEEKNIKEFLQSFVHIVQMFINTSGGLSG |
| | (GG)1205_IgG4(C2 | RSNICPPCPAPEFLGGPSVFLFPPKPKDTLMISRTPEVTC |
| | 26) Fc Homo | VVVDVSQEDPEVQFNWYVDGVEVHNAKTKPREEQFN |
| | | STYRVVSVLTVLHQDWLNGKEYKCKVSNKGLPSSIEK |
| | | TISKAKGQPREPQVYTLPPSQEEMTKNQVSLTCLVKGF |
| | | YPSDIAVEWESNGQPENNYKTTPPVLDSDGSFFLYSRL |
| | | TVDKSRWQQGNVFSCSVMHEALHNHYTQKSLSLS |
| 399 | ProC3999 | KLTTVRGGGGSASHYFERGGLSGRSNIGGNWVNVISD |
| | | LKKIEDLIQSMHIDATLYTESDVHPSCKVTAMKCFLDE |
| | | LQVISDESGDASIHDTVENLIILANNSLSSNGNVTESGC |
| | | KECEELEEKNIKEFLQSFVHIVQMFINTSGGLSGRSNICP |
| | | PCPAPEFLGGPSVFLFPPKPKDTLMISRTPEVTCVVVDV |
| | | SQEDPEVQFNWYVDGVEVHNAKTKPREEQFNSTYRV |
| | | VSVLTVLHQDWLNGKEYKCKVSNKGLPSSIEKTISKAK |
| | | GQPREPQVYTLPPSQEEMTKNQVSLTCLVKGFYPSDIA |
| | | VEWESNGQPENNYKTTPPVLDSDGSFFLYSRLTVDKSR |
| | | WQQGNVFSCSVMHEALHNHYTQKSLSLS |
| 400 | ProC4000 | ALTTVDGGGGSASHYFERGGLSGRSNIGGNWVNVISD |
| | | LKKIEDLIQSMHIDATLYTESDVHPSCKVTAMKCFLNE |
| | | LQVISNESGDASIHDTVENLIILANNSLSSNGNVTESGC |
| | | KECEELEEKNIKEFLQSFVHIVQMFINTSGGLSGRSNICP |
| | | PCPAPEFLGGPSVFLFPPKPKDTLMISRTPEVTCVVVDV |
| | | SQEDPEVQFNWYVDGVEVHNAKTKPREEQFNSTYRV |
| | | VSVLTVLHQDWLNGKEYKCKVSNKGLPSSIEKTISKAK |
| | | GQPREPQVYTLPPSQEEMTKNQVSLTCLVKGFYPSDIA |
| | | VEWESNGQPENNYKTTPPVLDSDGSFFLYSRLTVDKSR |
| | | WQQGNVFSCSVMHEALHNHYTQKSLSLS |
| 402 | Mutant of SEQ ID | NWVNVISDLKKIEDLIQSMHIDATLYTESDVHPSCKVT |
| | NO: 348 (L45 is | AMKCFLX1ELQVISX2ESGDASIHDTVENLIILANNSLSS |
| | mutated; L52 is | NGNVTESGCKECEELEEKNIKEFLQSFVHIVQMFINTS |
| | mutated) | where: |
| | | X1 is any amino acid that is not L; and |
| | | X2 is any amino acid |
| 403 | Mutant of SEQ ID | NWVNVISDLKKIEDLIQSMHIDATLYTESDVHPSCKVT |
| | NO: 348 (L45 is | AMKCFLX1ELQVISX2ESGDASIHDTVENLIILANNSLSS |
| | mutated to D, N, or | NGNVTESGCKECEELEEKNIKEFLQSFVHIVQMFINTS |
| | T; L52 is mutated) | where: |
| | | X1 is D, N, or T; and |
| | | X2 is any amino acid |
| 404 | Mutant of SEQ ID | NWVNVISDLKKIEDLIQSMHIDATLYTESDVHPSCKVT |
| | NO: 348 (L45 is | AMKCFLX1ELQVISLESGDASIHDTVENLIILANNSLSS |
| | mutated; L52 is not | NGNVTESGCKECEELEEKNIKEFLQSFVHIVQMFINTS |
| | mutated) | where: |
| | | X1 is any amino acid that is not L |
| 405 | Mutant of SEQ ID | NWVNVISDLKKIEDLIQSMHIDATLYTESDVHPSCKVT |
| | NO: 348 (L45D) | AMKCFLX1ELQVISLESGDASIHDTVENLIILANNSLSS |
| | | NGNVTESGCKECEELEEKNIKEFLQSFVHIVQMFINTS |
| | | where: |
| | | X1 is D |
| 406 | Mutant of SEQ ID | NWVNVISDLKKIEDLIQSMHIDATLYTESDVHPSCKVT |
| | NO: 348 (L45N) | AMKCFLX1ELQVISLESGDASIHDTVENLIILANNSLSS |
| | | NGNVTESGCKECEELEEKNIKEFLQSFVHIVQMFINTS |
| | | where: |
| | | X1 is N |
| 407 | Mutant of SEQ ID | NWVNVISDLKKIEDLIQSMHIDATLYTESDVHPSCKVT |
| | NO: 348 (L45T) | AMKCFLX1ELQVISLESGDASIHDTVENLIILANNSLSS |
| | | NGNVTESGCKECEELEEKNIKEFLQSFVHIVQMFINTS |
| | | where: |
| | | X1 is T |
| 408 | Mutant of SEQ ID | NWVNVISDLKKIEDLIQSMHIDATLYTESDVHPSCKVT |
| | NO: 348 (L45 is | AMKCFLX1ELQVISX2ESGDASIHDTVENLIILANNSLSS |
| | mutated; L52 is | NGNVTESGCKECEELEEKNIKEFLQSFVHIVQMFINTS |
| | mutated) | where: |
| | | X1 is any amino acid that is not L; and |
| | | X2 is any amino acid that is not L |
| 409 | Mutant of SEQ ID | NWVNVISDLKKIEDLIQSMHIDATLYTESDVHPSCKVT |
| | NO: 348 (L45 is | AMKCFLX1ELQVISX2ESGDASIHDTVENLIILANNSLSS |
| | mutated to D, N, or | NGNVTESGCKECEELEEKNIKEFLQSFVHIVQMFINTS |
| | T; L52 is mutated) | where: |
| | | X1 is D, N, or T; and |
| | | X2 is any amino acid that is not L |
| 410 | Mutant of SEQ ID | NWVNVISDLKKIEDLIQSMHIDATLYTESDVHPSCKVT |
| | NO: 348 (L45 is | AMKCFLX1ELQVISX2ESGDASIHDTVENLIILANNSLSS |
| | mutated to D, N, or | NGNVTESGCKECEELEEKNIKEFLQSFVHIVQMFINTS |
| | T; L52 is mutated | where: |
| | to D, N, or T) | X1 is D, N, or T; and |
| | | X2 is D, N, or T |
| 411 | Mutant of SEQ ID | NWVNVISDLKKIEDLIQSMHIDATLYTESDVHPSCKVT |
| | NO: 348 | AMKCFLX1ELQVISX2ESGDASIHDTVENLIILANNSLSS |
| | (L45D/L52D) | NGNVTESGCKECEELEEKNIKEFLQSFVHIVQMFINTS |
| | | where: |
| | | X1 is D; and X2 is D |
| 412 | Mutant of SEQ ID | NWVNVISDLKKIEDLIQSMHIDATLYTESDVHPSCKVT |
| | NO: 348 | AMKCFLX1ELQVISX2ESGDASIHDTVENLIILANNSLSS |
| | (L45D/L52N) | NGNVTESGCKECEELEEKNIKEFLQSFVHIVQMFINTS |
| | | where: |
| | | X1 is D; and |
| | | X2 is N |
| 413 | Mutant of SEQ ID | NWVNVISDLKKIEDLIQSMHIDATLYTESDVHPSCKVT |
| | NO: 348 | AMKCFLX1ELQVISX2ESGDASIHDTVENLIILANNSLSS |
| | (L45D/L52T) | NGNVTESGCKECEELEEKNIKEFLQSFVHIVQMFINTS |
| | | where: |
| | | X1 is D; and |
| | | X2 is T |
| 414 | Mutant of SEQ ID | NWVNVISDLKKIEDLIQSMHIDATLYTESDVHPSCKVT |
| | NO: 348 | AMKCFLX1ELQVISX2ESGDASIHDTVENLIILANNSLSS |
| | (L45N/L52D) | NGNVTESGCKECEELEEKNIKEFLQSFVHIVQMFINTS |
| | | where: |
| | | X1 is N; and |
| | | X2 is D |
| 415 | Mutant of SEQ ID | NWVNVISDLKKIEDLIQSMHIDATLYTESDVHPSCKVT |
| | NO: 348 | AMKCFLX1ELQVISX2ESGDASIHDTVENLIILANNSLSS |
| | (L45N/L52N) | NGNVTESGCKECEELEEKNIKEFLQSFVHIVQMFINTS |
| | | where: |
| | | X1 is N; and |
| | | X2 is N |
| 416 | Mutant of SEQ ID | NWVNVISDLKKIEDLIQSMHIDATLYTESDVHPSCKVT |
| | NO: 348 | AMKCFLX1ELQVISX2ESGDASIHDTVENLIILANNSLSS |
| | (L45N/L52T) | NGNVTESGCKECEELEEKNIKEFLQSFVHIVQMFINTS |
| | | where: |
| | | X1 is N; and |
| | | X2 is T |
| 417 | Mutant of SEQ ID | NWVNVISDLKKIEDLIQSMHIDATLYTESDVHPSCKVT |
| | NO: 348 | AMKCFLX1ELQVISX2ESGDASIHDTVENLIILANNSLSS |
| | (L45T/L52D) | NGNVTESGCKECEELEEKNIKEFLQSFVHIVQMFINTS |
| | | where: |
| | | X1 is T; and |
| | | X2 is D |
| 418 | Mutant of SEQ ID | NWVNVISDLKKIEDLIQSMHIDATLYTESDVHPSCKVT |
| | NO: 348 | AMKCFLX1ELQVISX2ESGDASIHDTVENLIILANNSLSS |
| | (L45T/L52N) | NGNVTESGCKECEELEEKNIKEFLQSFVHIVQMFINTS |
| | | where: |
| | | X1 is T; and |
| | | X2 is N |
| 419 | Mutant of SEQ ID | NWVNVISDLKKIEDLIQSMHIDATLYTESDVHPSCKVT |
| | NO: 348 | AMKCFLX1ELQVISX2ESGDASIHDTVENLIILANNSLSS |
| | (L45T/L52T) | NGNVTESGCKECEELEEKNIKEFLQSFVHIVQMFINTS |
| | | where: |
| | | X1 is T; and |
| | | X2 is T |
| 420 | Mutant of SEQ ID | NWVNVISDLKKIEDLIQSMHIDATLYTESDVHPSCKVT |
| | NO: 348 (L52D) | AMKCFLLELQVISX2ESGDASIHDTVENLIILANNSLSS |
| | | NGNVTESGCKECEELEEKNIKEFLQSFVHIVQMFINTS |
| | | Where: |
| | | X2 is D |
| 421 | Mutant of SEQ ID | NWVNVISDLKKIEDLIQSMHIDATLYTESDVHPSCKVT |
| | NO: 348 (L52N) | AMKCFLLELQVISX2ESGDASIHDTVENLIILANNSLSS |
| | | NGNVTESGCKECEELEEKNIKEFLQSFVHIVQMFINTS |
| | | Where: |
| | | X2 is N |
| 422 | Mutant of SEQ ID | NWVNVISDLKKIEDLIQSMHIDATLYTESDVHPSCKVT |
| | NO: 348 (L52T) | AMKCFLLELQVISX2ESGDASIHDTVENLIILANNSLSS |
| | | NGNVTESGCKECEELEEKNIKEFLQSFVHIVQMFINTS |
| | | Where: |
| | | X2 is T |
| 423 | ProC2972 | NWVNVISDLKKIEDLIQSMHIDATLYTESDVHPSCKVT |
| | L45D | AMKCFLDELQVISLESGDASIHDTVENLIILANNSLSSN |
| | | GNVTESGCKECEELEEKNIKEFLQSFVHIVQMFINTSG |
| | | GLSGRSNICPPCPAPEFLGGPSVFLFPPKPKDTLMISRT |
| | | PEVTCVVVDVSQEDPEVQFNWYVDGVEVHNAKTKPR |
| | | EEQFNSTYRVVSVLTVLHQDWLNGKEYKCKVSNKGLP |
| | | SSIEKTISKAKGQPREPQVYTLPPSQEEMTKNQVSLTC |
| | | LVKGFYPSDIAVEWESNGQPENNYKTTPPVLDSDGSFF |
| | | LYSRLTVDKSRWQQGNVFSCSVMHEALHNHYTQKSL |
| | | SLS |
| 424 | ProC2974 | NWVNVISDLKKIEDLIQSMHIDATLYTESDVHPSCKVT |
| | L45N | AMKCFLNELQVISLESGDASIHDTVENLIILANNSLSSN |
| | | GNVTESGCKECEELEEKNIKEFLQSFVHIVQMFINTS |
| | | GGLSGRSNICPPCPAPEFLGGPSVFLFPPKPKDTLMISRT |
| | | PEVTCVVVDVSQEDPEVQFNWYVDGVEVHNAKTKPR |
| | | EEQFNSTYRVVSVLTVLHQDWLNGKEYKCKVSNKGL |
| | | PSSIEKTISKAKGQPREPQVYTLPPSQEEMTKNQVSLTC |
| | | LVKGFYPSDIAVEWESNGQPENNYKTTPPVLDSDGSFF |
| | | LYSRLTVDKSRWQQGNVFSCSVMHEALHNHYTQKSL |
| | | SLS |
| 425 | ProC2976 | NWVNVISDLKKIEDLIQSMHIDATLYTESDVHPSCKVT |
| | L45T | AMKCFLTELQVISLESGDASIHDTVENLIILANNSLSSN |
| | | GNVTESGCKECEELEEKNIKEFLQSFVHIVQMFINTS |
| | | GGLSGRSNICPPCPAPEFLGGPSVFLFPPKPKDTLMISRT |
| | | PEVTCVVVDVSQEDPEVQFNWYVDGVEVHNAKTKPR |
| | | EEQFNSTYRVVSVLTVLHQDWLNGKEYKCKVSNKGL |
| | | PSSIEKTISKAKGQPREPQVYTLPPSQEEMTKNQVSLTC |
| | | LVKGFYPSDIAVEWESNGQPENNYKTTPPVLDSDGSFF |
| | | LYSRLTVDKSRWQQGNVFSCSVMHEALHNHYTQKSL |
| | | SLS |
| 426 | ProC2978 | NWVNVISDLKKIEDLIQSMHIDATLYTESDVHPSCKVT |
| | L52D | AMKCFLLELQVISDESGDASIHDTVENLIILANNSLSSN |
| | | GNVTESGCKECEELEEKNIKEFLQSFVHIVQMFINTS |
| | | GGLSGRSNICPPCPAPEFLGGPSVFLFPPKPKDTLMISRT |
| | | PEVTCVVVDVSQEDPEVQFNWYVDGVEVHNAKTKPR |
| | | EEQFNSTYRVVSVLTVLHQDWLNGKEYKCKVSNKGL |
| | | PSSIEKTISKAKGQPREPQVYTLPPSQEEMTKNQVSLTC |
| | | LVKGFYPSDIAVEWESNGQPENNYKTTPPVLDSDGSFF |
| | | LYSRLTVDKSRWQQGNVFSCSVMHEALHNHYTQKSL |
| | | SLS |
| 427 | ProC2979 | NWVNVISDLKKIEDLIQSMHIDATLYTESDVHPSCKVT |
| | L52N | AMKCFLLELQVISNESGDASIHDTVENLIILANNSLSSN |
| | | GNVTESGCKECEELEEKNIKEFLQSFVHIVQMFINTS |
| | | GGLSGRSNICPPCPAPEFLGGPSVFLFPPKPKDTLMIS |
| | | RTPEVTCVVVDVSQEDPEVQFNWYVDGVEVHNAKTKPR |
| | | EEQFNSTYRVVSVLTVLHQDWLNGKEYKCKVSNKGL |
| | | PSSIEKTISKAKGQPREPQVYTLPPSQEEMTKNQVSLTC |
| | | LVKGFYPSDIAVEWESNGQPENNYKTTPPVLDSDGSFF |
| | | LYSRLTVDKSRWQQGNVFSCSVMHEALHNHYTQKSL |
| | | SLS |
| 428 | ProC2980 | NWVNVISDLKKIEDLIQSMHIDATLYTESDVHPSCKVT |
| | L52T | AMKCFLLELQVISTESGDASIHDTVENLIILANNSLSSN |
| | | GNVTESGCKECEELEEKNIKEFLQSFVHIVQMFINTS |
| | | GGLSGRSNICPPCPAPEFLGGPSVFLFPPKPKDTLMIS |
| | | RTPEVTCVVVDVSQEDPEVQFNWYVDGVEVHNAKTKPR |
| | | EEQFNSTYRVVSVLTVLHQDWLNGKEYKCKVSNKGL |
| | | PSSIEKTISKAKGQPREPQVYTLPPSQEEMTKNQVSLTC |
| | | LVKGFYPSDIAVEWESNGQPENNYKTTPPVLDSDGSFF |
| | | LYSRLTVDKSRWQQGNVFSCSVMHEALHNHYTQKSL |
| | | SLS |
| 429 | ProC2973 | NWVNVISDLKKIEDLIQSMHIDATLYTESDVHPSCKVT |
| | L45D/L52D | AMKCFLDELQVISDESGDASIHDTVENLIILANNSLSSN |
| | | GNVTESGCKECEELEEKNIKEFLQSFVHIVQMFINTS |
| | | GGLSGRSNICPPCPAPEFLGGPSVFLFPPKPKDTLMISRT |
| | | PEVTCVVVDVSQEDPEVQFNWYVDGVEVHNAKTKPR |
| | | EEQFNSTYRVVSVLTVLHQDWLNGKEYKCKVSNKGL |
| | | PSSIEKTISKAKGQPREPQVYTLPPSQEEMTKNQVSLTC |
| | | LVKGFYPSDIAVEWESNGQPENNYKTTPPVLDSDGSFF |
| | | LYSRLTVDKSRWQQGNVFSCSVMHEALHNHYTQKSL |
| | | SLS |
| 430 | ProC2975 | NWVNVISDLKKIEDLIQSMHIDATLYTESDVHPSCKVT |
| | L45N/L52N | AMKCFLNELQVISNESGDASIHDTVENLIILANNSLSSN |
| | | GNVTESGCKECEELEEKNIKEFLQSFVHIVQMFINTS |
| | | GGLSGRSNICPPCPAPEFLGGPSVFLFPPKPKDTLMISRT |
| | | PEVTCVVVDVSQEDPEVQFNWYVDGVEVHNAKTKPR |
| | | EEQFNSTYRVVSVLTVLHQDWLNGKEYKCKVSNKGL |
| | | PSSIEKTISKAKGQPREPQVYTLPPSQEEMTKNQVSLTC |
| | | LVKGFYPSDIAVEWESNGQPENNYKTTPPVLDSDGSFF |
| | | LYSRLTVDKSRWQQGNVFSCSVMHEALHNHYTQKSL |
| | | SLS |
| 431 | ProC2977 | NWVNVISDLKKIEDLIQSMHIDATLYTESDVHPSCKVT |
| | L45T/L52T | AMKCFLTELQVISTESGDASIHDTVENLIILANNSLSSN |
| | | GNVTESGCKECEELEEKNIKEFLQSFVHIVQMFINTS |
| | | GGLSGRSNICPPCPAPEFLGGPSVFLFPPKPKDTLMIS |
| | | RTPEVTCVVVDVSQEDPEVQFNWYVDGVEVHNAKTKPR |
| | | EEQFNSTYRVVSVLTVLHQDWLNGKEYKCKVSNKGL |
| | | PSSIEKTISKAKGQPREPQVYTLPPSQEEMTKNQVSLTC |
| | | LVKGFYPSDIAVEWESNGQPENNYKTTPPVLDSDGSFF |
| | | LYSRLTVDKSRWQQGNVFSCSVMHEALHNHYTQKSL |
| | | SLS |
| 500 | human IL12RB | MAAPALSWRLPLLILLLPLATSWASAAVNGTSQFTCFY |
| | (P14784) | NSRANISCVWSQDGALQDTSCQVHAWPDRRRWNQTC |
| | | ELLPVSQASWACNLILGAPDSQKLTTVDIVTLRVLCRE |
| | | GVRWRVMAIQDFKPFENLRLMAPISLQVVHVETHRCN |
| | | ISWEISQASHYFERHLEFEARTLSPGHTWEEAPLLTLKQ |
| | | KQEWICLETLTPDTQYEFQVRVKPLQGEFTTWSPWSQP |
| | | LAFRTKPAALGKDTIPWLGHLLVGLSGAFGFIILVYLLI |
| | | NCRNTGPWLKKVLKCNTPDPSKFFSQLSSEHGGDVQK |
| | | WLSSPFPSSSFSPGGLAPEISPLEVLERDKVTQLLLQQD |
| | | KVPEPASLSSNHSLTSCFTNQGYFFFHLPDALEIEACQV |
| | | YFTYDPYSEEDPDEGVAGAPTGSSPQPLQPLSGEDDAY |
| | | CTFPSRDDLLLFSPSLLGGPSPPSTAPGGSGAGEERMPP |
| | | SLQERVPRDWDPQPLGPPTPGVPDLVDFQPPPELVLRE |
| | | AGEEVPDAGPREGVSFPWSRPPGQGEFRALNARLPLNT |
| | | DAYLSLQELQGQDPTHLV |
| 501 | MM | ALTTVX-linker-ASHYFE(R/K) |
| | | Where |
| | | X is D, K, or R, and |
| | | the linker consists of 1 to 20 amino acids |
| 502 | MM | ALTTVD-linker-ASHYFE(R/K) |
| | | Where |
| | | the linker consists of 1 to 20 amino acids |
| 503 | MM | ALTTVK-linker-ASHYFE(R/K) |
| | | Where |
| | | the linker consists of 1 to 20 amino acids |
| 504 | MM | ALTTVR-linker-ASHYFE(R/K) |
| | | Where |
| | | the linker consists of 1 to 20 amino acids |
| 505 | MM | ALTTVDGGGGSASHYFE(R/K) |
| 506 | MM | ALTTVKGGGGSASHYFE(R/K) |
| 507 | MM | ALTTVRGGGGSASHYFE(R/K) |
| 508 | MM | ALTTVX-linker-ASHYFE |
| | | Where |
| | | X is D, K, or R, and |
| | | the linker consists of 1 to 20 amino acids |
| 509 | MM | ALTTVD-linker-ASHYFE |
| | | Where |
| | | the linker consists of 1 to 20 amino acids |
| 510 | MM | ALTTVK-linker-ASHYFE |
| | | Where |
| | | the linker consists of 1 to 20 amino acids |
| 511 | MM | ALTTVR-linker-ASHYFE |
| | | Where |
| | | the linker consists of 1 to 20 amino acids |
| 512 | MM | ALTTVDGGGGSASHYFE |
| 513 | MM | ALTTVKGGGGSASHYFE |
| 514 | MM | ALTTVRGGGGSASHYFE |
| 515 | MM | X₁LTTVX₂-linker-ASHYFE |
| | | Where |
| | | X₁ is absent or any amino acid, |
| | | X₂ is D, K, or R, and |
| | | the linker consists of 1 to 20 amino acids |
| 516 | MM | X₁LTTVD-linker-ASHYFE |
| | | Where X₁ is absent or any amino acid, |
| | | the linker consists of 1 to 20 amino acids |
| 517 | MM | X₁LTTVK-linker-ASHYFE |
| | | Where X₁ is absent or any amino acid, |
| | | the linker consists of 1 to 20 amino acids |
| 518 | MM | X₁LTTVR-linker-ASHYFE |
| | | Where X₁ is absent or any amino acid, |
| | | the linker consists of 1 to 20 amino acids |
| 600 | MM | X₁LTTVDGGGGSASHYFE |
| | | Where X₁ is absent or any amino acid |
| 601 | MM | X₁LTTVKGGGGSASHYFE |
| | | Where X₁ is absent or any amino acid |
| 602 | MM | X₁LTTVRGGGGSASHYFE |
| | | Where X₁ is absent or any amino acid |
| 520 | Sushi domain of | ITCPPPMSVEHADIWVKSYSLYSRERYICNSGFKRKAG |
| | human IL15RA | TSSLTECVLNKATNVAHWTTPSLKCIR |
| | (Q13261) residue | |
| | 31-95 | |
| 521 | Human IL-21 | MRSSPGNMERIVICLMVIFLGTLVHKSSSQGQDRHMIR |
| | (Q9HBE4) | MRQLIDIVDQLKNYVNDLVPEFLPAPEDVETNCEWSAF |
| | | SCFQKAQLKSANTGNNERIINVSIKKLKRKPPSTNAGR |
| | | RQKHRLTCPSCDSYEKKPPKEFLERFKSLLQKMIHQHL |
| | | SSRTHGSEDS |
| 522 | Mouse IL-21 | MERTLVCLVVIFLGTVAHKSSPQGPDRLLIRLRHLIDI |
| | (Q9ES17) | VEQLKIYENDLDPELLSAPQDVKGHCEHAAFACFQKAK |
| | | LKPSNPGNNKTFIIDLVAQLRRRLPARRGGKKQKHIAK |
| | | CPSCDSYEKRTPKEFLERLKWLLQKMIHQHLS |
| 523 | ProC2970 | ALTTVDGGGGSASHYFERGGLSGRSNIGGNWVNVISD |
| | | LKKIEDLIQSMHIDATLYTESDVHPSCKVTAMKCFLLE |
| | | LQVISLESGDASIHDTVENLIILANNSLSSNGNVTESGC |
| | | KECEELEEKNIKEFLQSFVHIVQMFINTSGGLSGRSNICP |
| | | PCPAPEFLGGPSVFLFPPKPKDTLMISRTPEVTCVVVDV |
| | | SQEDPEVQFNWYVDGVEVHNAKTKPREEQFNSTYRV |
| | | VSVLTVLHQDWLNGKEYKCKVSNKGLPSSIEKTISKAK |
| | | GQPREPQVYTLPPSQEEMTKNQVSLTCLVKGFYPSDIA |
| | | VEWESNGQPENNYKTTPPVLDSDGSFFLYSRLTVDKSR |
| | | WQQGNVFSCSVMHEALHNHYTQKSLSLS |
| 524 | ProC2970 with | METDTLLLWVLLLWVPGSTGALTTVDGGGGSASHYFER |
| | signal sequence | GGLSGRSNIGGNWVNVISDLKKIEDLIQSMHIDATLYT |
| | | ESDVHPSCKVTAMKCFLLELQVISLESGDASIHDTVEN |
| | | LIILANNSLSSNGNVTESGCKECEELEEKNIKEFLQSF |
| | | VHIVQMFINTSGGLSGRSNICPPCPAPEFLGGPSVFLF |
| | | PPKPKDTLMISRTPEVTCVVVDVSQEDPEVQFNWYVDG |
| | | VEVHNAKTKPREEQFNSTYRVVSVLTVLHQDWLNGKEY |
| | | KCKVSNKGLPSSIEKTISKAKGQPREPQVYTLPPSQEE |
| | | MTKNQVSLTCLVKGFYPSDIAVEWESNGQPENNYKTTP |
| | | PVLDSDGSFFLYSRLTVDKSRWQQGNVFSCSVMHEALH |
| | | NHYTQKSLSLS |
| 525 | ProC2982 | ALTTVDGGGGSASHYFERGGLSGRSNIGGNWVNVISD |
| | Beta- | LKKIEDLIQSMHIDATLYTESDVHPSCKVTAMKCFLLE |
| | peptide_CM_IL- | LQVISLESGDASIHDTVENLIILANNSLSSNGNVTESGC |
| | 15_linker_CM_IgG | KECEELEEKNIKEFLQSFVHIVQMFINTSGGLSGRSNICP |
| | 4(C226) | PCPAPEFLGGPSVFLFPPKPKDTLMISRTPEVTCVVVDV |
| | | SQEDPEVQFNWYVDGVEVHNAKTKPREEQFNSTYRV |
| | | VSVLTVLHQDWLNGKEYKCKVSNKGLPSSIEKTISKAK |
| | | GQPREPQVYTLPPSQEEMTKNQVSLTCLVKGFYPSDIA |
| | | VEWESNGQPENNYKTTPPVLDSDGSFFLYSRLTVDKSR |
| | | WQQGNVFSCSVMHEALHNHYTQKSLSLS |
| 526 | ProC2982 | ITCPPPMSVEHADIWVKSYSLYSRERYICNSGFKRKAG |
| | Sushi domain | TSSLTECVLNKATNVAHWTTPSLKCIRDPALVHQRPAP |
| | (underlined) | PGGGGSGGGGSHHHHHHHHHH |
| | with_His-tag | |
| 527 | ProC3571 | ALTTVDGGGGSASHYFERGGLSGRSNIGGNWVNVISD |
| | Beta-peptide_IL- | LKKIEDLIQSMHIDATLYTESDVHPSCKVTAMKCFLLE |
| | 15_linker CM G4 | LQVISLESGDASIHDTVENLIILANNSLSSNGNVTESGC |
| | K | KECEELEEKNIKEFLQSFVHIVQMFINTSGGLSGRSNICP |
| | | PCPAPEFEGGPSVFLFPPKPKDTLMISRTPEVTCVVVDV |
| | | SQEDPEVQFNWYVDGVEVHNAKTKPREEQFNSTYRV |
| | | VSVLTVLHQDWLNGKEYKCKVSNKGLPSSIEKTISKAK |
| | | GQPREPQVYTLPPCQEEMTKNQVSLWCLVKGFYPSDI |
| | | AVEWESNGQPENNYKTTPPVLDSDGSFFLYSRLTVDKS |
| | | RWQEGNVFSCSVMHEALHNRFTQKSLSLSLGK |
| 528 | ProC3571 | ITCPPPMSVEHADIWVKSYSLYSRERYICNSGFKRKAG |
| | Sushi | TSSLTECVLNKATNVAHWTTPSLKCIRGGLSGRSNIES |
| | domain(underlined) | KYGPPCPPCPAPEFEGGPSVFLFPPKPKDTLMISRTPEVT |
| | linker CM | CVVVDVSQEDPEVQFNWYVDGVEVHNAKTKPREEQF |
| | (bold)_G4H | NSTYRVVSVLTVLHQDWLNGKEYKCKVSNKGLPSSIE |
| | | KTISKAKGQPREPQVCTLPPSQEEMTKNQVSLSCAVKG |
| | | FYPSDIAVEWESNGQPENNYKTTPPVLDSDGSFFLVSR |
| | | LTVDKSRWQEGNVFSCSVMHEALHNHYTQKSLSLSLG |
| | | K |
| 529 | Polynucleotide | atggaaaccgacacactgctgctgtgggtgctgcttttgtgggtgc |
| | encoding ProC2970 | caggatcCACAGGCGCCCTGACAACAGTTGATGGTGGCGGAGGAA |
| | with signal | GCGCCAGCCACTACTTTGAAAGAGGCGGCCTGAGCG |
| | sequence (signal | GCAGAAGCAACATCGGCGGAAATTGGGTCAACGTG |
| | sequence is | ATCAGCGACCTGAAGAAGATCGAGGACCTGATCCAG |
| | underlined) | AGCATGCACATCGACGCCACACTGTACACCGAGAGC |
| | | GACGTGCACCCTAGCTGTAAAGTGACCGCCATGAAG |
| | | TGCTTTCTGCTGGAACTGCAAGTGATCAGCCTGGAA |
| | | AGCGGCGACGCCAGCATCCACGACACCGTGGAAAA |
| | | CCTGATCATCCTGGCCAACAACAGCCTGAGCAGCAA |
| | | CGGCAATGTGACCGAGTCCGGCTGCAAAGAGTGCGA |
| | | GGAACTGGAAGAGAAGAATATCAAAGAGTTCCTGC |
| | | AGAGCTTCGTGCACATCGTGCAGATGTTCATCAACA |
| | | CCAGCGGCGGACTGTCCGGCCGGTCCAATATTTGTC |
| | | CTCCTTGTCCTGCTCCTGAGTTCCTCGGCGGACCTTC |
| | | CGTGTTCCTGTTTCCTCCAAAGCCTAAGGACACCCTG |
| | | ATGATCAGCagaacccctgaagtgacctgcgtggtggtcgacgtttcacaaga |
| | | ggaccccgaggtgcagttcaattggtacgtggacggcgtggaagtgcacaacgccaa |
| | | gaccaagcctagagaggaacagttcaacagcacctacagagtggtgtccgtgctgac |
| | | cgtgctgcaccaggattggctgaacggcaaagagtacaagtgcaaggtgtccaacaa |
| | | gggcctgcctagcagcatcgagaaaaccatcagcaaggccaagggccagccaagg |
| | | gaaccccaggtttacacactgccacctagccaagaggaaatgaccaagaaccaggtg |
| | | tccctgacctgcctggtcaagggcttttacccctccgatatcgccgtggaatgggagag |
| | | caatggccagcctgagaacaactacaagaccacacctcctgtgctggacagcgacgg |
| | | ctcattcttcctgtacagcagactgaccgtggacaagagcagatggcagcagggcaa |
| | | cgtgttcagctgcagcgtgatgcacgaggccctgcacaaccactacacccagaagtct |
| | | ctgagcctgagctga |
| 722 | MM | KLTTVX-linker-ASHYFE(R/K) |
| | | Where X is D or R, and |
| | | the linker consists of 1 to 20 amino acids |
| 723 | MM | KLTTVD-linker-ASHYFE(R/K) |
| | | Where the linker consists of 1 to 20 amino acids |
| 724 | MM | KLTTVR-linker-ASHYFE(R/K) |
| | | Where the linker consists of 1 to 20 amino acids |
| 725 | MM | KLTTVDGGGGSASHYFE(R/K) |
| 726 | MM | KLTTVRGGGGSASHYFE(R/K) |
| 727 | MM | SQKLTTVX-linker-ASHYFERHLE |
| | | Where X is D or R, and the linker consists of 1 to 20 amino |
| | | acids |
| 728 | MM | SQKLTTVD-linker-ASHYFERHLE |
| | | Where the linker consists of 1 to 20 amino acids |
| 729 | MM | SQKLTTVR-linker-ASHYFERHLE |
| | | Where the linker consists of 1 to 20 amino acids |
| 730 | MM | SQKLTTVDGGGGSASHYFERHLE |
| 731 | MM | SQKLTTVRGGGGSASHYFERHLE |
| 732 | MM | SQALTTVRGGGGSASHYFERHLE |
| 733 | MM | SQALTTVDGGGGSASHYFERHLE |

### Numbered Items

The present disclosure includes the following non-limiting items:
1. An isolated polypeptide comprising amino acid sequence X₁LTTVX₂-linker-ASHYFE (SEQ ID NO: 515) (MM), wherein X₁ is absent or any amino acid, wherein X₂ is D, K, or R, and wherein the linker consists of 1 to 20 amino acids.
2. The isolated polypeptide of item 1, comprising amino acid sequence ALTTVX-linker-ASHYFE (SEQ ID NO: 508) (MM), wherein X is D, K, or R, and wherein the N-terminal alanine residue is optionally substituted by any other amino acid, and the linker consists of 1 to 20 amino acids.
3. The isolated polypeptide of items 1 or 2, wherein the X₂ or the X is D.
4. The isolated polypeptide of items 1 or 2, wherein the X₂ or the X is K.
5. The isolated polypeptide of items 1 or 2, wherein the X₂ or the X is R.
6. The isolated polypeptide of items 1, wherein the X₁ is A.
7. The isolated polypeptide of items 1 or 2, wherein the X₁ is K or the N-terminal alanine residue is substituted with K.
8. The isolated polypeptide of any one or combination of preceding items, wherein the linker consists of 4, 5, 6, 7, or 8 amino acids.
9. The isolated polypeptide of any one or combination of preceding items, wherein the linker consists of 6 amino acids.
10. The isolated polypeptide of any one or combination of preceding items, wherein the linker is selected from the group consisting of SEQ ID NOs: 2, 210-235, 245, 250, and 318-335.
11. The isolated polypeptide of any one of items 1-7, wherein the linker is GGGGS (SEQ ID NO: 216).
12. The isolated polypeptide of any one or combination of items 1-2, wherein the N-terminus, the C-terminus, or both of the isolated polypeptide is extended by 1, 2, 3, 4, 5, 6, 7, 8, 9, 10, 11, 12, 13, 14, 15, 16, 17, 18, 19, or 20 amino acids.
13. The isolated polypeptide of item 12, wherein the amino acid sequence comprises ALTTVDGGGGSASHYFER (SEQ ID NO: 236) or ALTTVDGGGGSASHYFEK (SEQ ID NO: 237), or wherein the N-terminal alanine residue is optionally absent or substituted by any other amino acid.
14. The isolated polypeptide of item 12, wherein the amino acid sequence comprises ALTTVKGGGGSASHYFER (SEQ ID NO: 238) or ALTTVKGGGGSASHYFEK (SEQ ID NO: 239), or wherein the N-terminal alanine residue is optionally absent or substituted by any other amino acid.
15. The isolated polypeptide of item 12, wherein the amino acid sequence comprises ALTTVRGGGGSASHYFER (SEQ ID NO: 240) or ALTTVRGGGGSASHYFEK (SEQ ID NO: 241), or wherein the N-terminal alanine residue is optionally absent or substituted by any other amino acid.
16. The isolated polypeptide of item 12, wherein the isolated polypeptide comprises a sequence selected from SQKLTTVDGGGGSASHYFERHLE (SEQ ID NO: 730), SQKLTTVRGGGGSASHYFERHLE (SEQ ID NO: 731), SQALTTVRGGGGSASHYFERHLE (SEQ ID NO: 732), or SQALTTVDGGGGSASHYFERHLE (SEQ ID NO: 733).
17. The isolated polypeptide of any one or combination of items 1-16, wherein the isolated polypeptide comprises a cytokine.
18. The isolated polypeptide of any one or combination of items 1-16, wherein the isolated polypeptide is disposed in a complex comprising two or more polypeptides, and wherein the complex comprises a cytokine.
19. The isolated polypeptide of item 18, wherein the cytokine is disposed in a polypeptide that is complexed with the isolated polypeptide.
20. The isolated polypeptide of any one or combination of items 17-19, wherein the amino acid sequence is a masking moiety that inhibits binding of the cytokine with its receptor.
21. The isolated polypeptide of any one or combination of items 17-20, wherein the cytokine is a cytokine that binds IL2/IL15 receptor beta and/or IL2/IL15 receptor gamma.
22. The isolated polypeptide of any one or combination of items 17-20, wherein the cytokine binds to IL-15Rα.
23. The isolated polypeptide of any one or combination of items 17-20, wherein the cytokine binds to IL-2Rα.
24. An activatable cytokine construct (ACC) comprising a cytokine polypeptide (CP), a cleavable moiety (CM), and the isolated polypeptide of any one or combination of items 1-16, wherein the MM is coupled to the CP via the CM and inhibits the binding of CP to its receptor.
25. The ACC of item 24, wherein the CP is an interleukin polypeptide.
26. The ACC of item 25, wherein the interleukin polypeptide comprises a sequence at least 85%, 90%, or 95% identical to SEQ ID NO: 348, 129, or 130.
27. The ACC of item 25, wherein the interleukin polypeptide comprises SEQ ID NO: 348, 129, or 130.
28. The ACC of item 25, wherein the interleukin polypeptide comprises a sequence at least 85%, 90%, or 95% identical to SEQ ID NO: 119 or 120.
29. The ACC of item 25, wherein the interleukin polypeptide comprises SEQ ID NO: 119 or 120.
30. The ACC of item 25, wherein the interleukin polypeptide comprises a sequence at least 85%, 90%, or 95% identical to SEQ ID NO: 121 or 122.
31. The ACC of item 25, wherein the interleukin polypeptide comprises SEQ ID NO: 121 or 122.
32. The ACC of item 25, wherein the interleukin polypeptide comprises a sequence at least 85%, 90%, or 95% identical to SEQ ID NO: 123 or 124.
33. The ACC of item 25, wherein the interleukin polypeptide comprises SEQ ID NO: 123 or 124.
34. The ACC of item 25, wherein the interleukin polypeptide comprises a sequence at least 85%, 90%, or 95% identical to SEQ ID NO: 125 or 126.
35. The ACC of item 25, wherein the interleukin polypeptide comprises SEQ ID NO: 125 or 126.
36. The ACC of item 25, wherein the interleukin polypeptide comprises a sequence at least 85%, 90%, or 95% identical to SEQ ID NO: 521 or 522.
37. The ACC of item 25, wherein the interleukin polypeptide comprises SEQ ID NO: 521 or 522.
38. The ACC of any one or combination of items 24-37, wherein the CM comprises no more than 8 amino acids.
39. The ACC of any one or combination of items 24-38, wherein the CM is cleavable by a urokinase (uPA) and/or a matrix metalloproteinase (MMP).
40. The ACC of any one or combination of items 24-39, further comprising a linker (L1) between the CM and the CP.
41. The ACC of any one or combination of items 24-39, further comprising a linker (L2) between the CM and the MM.
42. The ACC of any one or combination of items 24-41, further comprising a first linker (L1) between the CM and the CP and a second linker (L2) between the CM and the MM.
43. The ACC of any one or combination of items 24-42, further comprising a steric mask that further inhibits binding of the CP to its receptor.
44. An ACC comprising a first monomer construct and a second monomer construct, wherein
   the first monomer construct comprises a first cytokine polypeptide (CP1), a first cleavable moiety (CM1), a first dimerization domain (DD1) coupled to the CP1 via the CM1, and a first masking moiety (MM1),
   the second monomer construct comprises a second cytokine polypeptide (CP2), a second cleavable moiety (CM2), a second dimerization domain (DD2) coupled to the CP2 via the CM2, and a second masking moiety (MM2),
   the DD1 and DD2 bind each other thereby forming a dimer of the first and second monomer constructs, and
   the MM1 and/or the MM2 comprises the isolated polypeptide of any one or combination of items 1-11.
45. The ACC of item 44, wherein the first monomer construct comprises a third cleavable moiety (CM3) and the MM1 is coupled to the CP1 via the CM3.
46. The ACC of any one or combination of items 44-45, wherein the MM1 is coupled to the CP1 via the DD1 and the CM1.
47. The ACC of any one or combination of items 44-46, wherein the second monomer construct comprises a fourth cleavable moiety (CM4) and the MM2 is coupled to the CP2 via the CM4.
48. The ACC of any one or combination of items 44-47, wherein the MM2 is coupled to the CP2 via the DD2 and the CM2.
49. The ACC of any one or combination of items 44-48, each of the first monomer construct and the second monomer construct comprises a Linking Region comprising no more than 18 amino acids.
50. The ACC of any one or combination of items 44-49, wherein
   the CP1 and the CM1 directly abut each other, and/or
   the CM1 and the DD1 directly abut each other.
51. The ACC of any one or combination of items 44-50, wherein
   the CP2 and the CM2 directly abut each other, and/or
   the CM2 and the DD2 directly abut each other.
52. The ACC of any one or combination of items 44-51, wherein the CP1 comprises a sequence at least 85%, 90%, or 95% identical to SEQ ID NO: 348, 129, or 130.
53. The ACC of any one or combination of items 44-51, wherein the CP2 comprises a sequence at least 85%, 90%, or 95% identical to SEQ ID NO: 348, 129, or 130.
54. The ACC of any one or combination of items 44-51, wherein the CP1 comprises SEQ ID NO: 348, 129, or 130.
55. The ACC of any one or combination of items 44-51, wherein the CP2 comprises SEQ ID NO: 348, 129, or 130.
56. The ACC of any one or combination of items 44-51, wherein the CP1 comprises a sequence at least 85%, 90%, or 95% identical to SEQ ID NO: 119 or 120.
57. The ACC of any one or combination of items 44-51, wherein the CP2 comprises a sequence at least 85%, 90%, or 95% identical to SEQ ID NO: 119 or 120.
58. The ACC of any one or combination of items 44-51, wherein the CP1 comprises SEQ ID NO: 119 or 120.
59. The ACC of any one or combination of items 44-51, wherein the CP2 comprises SEQ ID NO: 119 or 120.
60. The ACC of any one or combination of items 44-51, wherein the CP1 comprises a sequence at least 85%, 90%, or 95% identical to SEQ ID NO: 121 or 122.
61. The ACC of any one or combination of items 44-51, wherein the CP2 comprises a sequence at least 85%, 90%, or 95% identical to SEQ ID NO: 121 or 122.
62. The ACC of any one or combination of items 44-51, wherein the CP1 comprises SEQ ID NO: 121 or 122.
63. The ACC of any one or combination of items 44-51, wherein the CP2 comprises SEQ ID NO: 121 or 122.
64. The ACC of any one or combination of items 44-51, wherein the CP1 comprises a sequence at least 85%, 90%, or 95% identical to SEQ ID NO: 123 or 124.
65. The ACC of any one or combination of items 44-51, wherein the CP2 comprises a sequence at least 85%, 90%, or 95% identical to SEQ ID NO: 123 or 124.
66. The ACC of any one or combination of items 44-51, wherein the CP1 comprises SEQ ID NO: 123 or 124.
67. The ACC of any one or combination of items 44-51, wherein the CP2 comprises SEQ ID NO: 123 or 124.
68. The ACC of any one or combination of items 44-51, wherein the CP1 comprises a sequence at least 85%, 90%, or 95% identical to SEQ ID NO: 125 or 126.
69. The ACC of any one or combination of items 44-51, wherein the CP2 comprises a sequence at least 85%, 90%, or 95% identical to SEQ ID NO: 125 or 126.
70. The ACC of any one or combination of items 44-51, wherein the CP1 comprises SEQ ID NO: 125 or 126.
71. The ACC of any one or combination of items 44-51, wherein the CP2 comprises SEQ ID NO: 125 or 126.
72. The ACC of any one or combination of items 44-51, wherein the CP1 comprises a sequence at least 85%, 90%, or 95% identical to SEQ ID NO: 521 or 522.
73. The ACC of any one or combination of items 44-51, wherein the CP2 comprises a sequence at least 85%, 90%, or 95% identical to SEQ ID NO: 521 or 522.
74. The ACC of any one or combination of items 44-51, wherein the CP1 comprises SEQ ID NO: 521 or 522.
75. The ACC of any one or combination of items 44-51, wherein the CP2 comprises SEQ ID NO: 521 or 522.
76. The ACC of any one or combination of items 44-75, wherein the first monomer construct and the second monomer construct are identical.
77. The ACC of any one or combination of items 44-76, wherein the first monomer construct and the second monomer construct have 95% sequence homology.
78. The ACC of any one or combination of items 44-77, further comprising a third monomer comprising a Sushi domain comprising of the sequence of SEQ ID NO: 520, wherein the third monomer is non-covalently or covalently bound to the ACC.
79. The ACC of any one or combination of items 44-78, further comprising a fourth monomer comprising a Sushi domain comprising of the sequence of SEQ ID NO: 520, wherein the fourth monomer is non-covalently or covalently bound to the ACC.
80. The ACC of any one or combination of items 78 or 79, wherein the third and/or fourth monomer further comprises a tag.
81. An ACC comprising a first monomer construct and a second monomer construct, wherein
   the first monomer construct comprises a first cytokine polypeptide (CP1), a first dimerization domain (DD1), and a first masking moiety (MM1),
   the second monomer construct comprises a second cytokine polypeptide (CP2), a first cleavable moiety (CM1), a second dimerization domain (DD2) coupled to the CP2 via the CM1, and a second masking moiety (MM2),
   the MM1 and/or the MM2 is the isolated polypeptide of any one or combination of items 1-11, and
   the DD1 and DD2 bind each other thereby forming a dimer of the first and second monomer constructs.
82. The ACC of item 81, wherein the first monomer construct further comprises a second cleavable moiety (CM2) and the MM1 is coupled to the CP1 via the CM2.
83. The ACC of any one or combination of items 81 or 82, wherein the MM2 is coupled to the CP2 via the DD2 and the CM1.
84. The ACC of any one or combination of items 81-83, wherein the second monomer construct further comprises a third cleavable moiety (CM3), wherein the MM2 is coupled to the CP2 via the CM3.
85. An ACC comprising a first monomer construct and a second monomer construct, wherein
   the first monomer construct comprises a first cytokine polypeptide (CP1), a first dimerization domain (DD1), and a first masking moiety (MM1),
   the second monomer construct comprises a second cytokine polypeptide (CP2), a second dimerization domain (DD2), and a second masking moiety (MM2),
   the CP1 and/or the CP2 comprises an amino acid sequence that functions as a substrate for a protease, and the DD1 and/or DD2 is coupled to the CP1 and/or CP2 via the amino acid sequence,
   the MM1 and/or the MM2 is the isolated polypeptide of any one or combination of items 1-11, and
   the DD1 and DD2 bind each other thereby forming a dimer of the first and second monomer constructs.
86. The ACC of item 85, wherein the CP1 comprises the amino acid sequence that functions as a substrate for a protease, and the MM1 is coupled to the CP1 via the amino acid sequence.
87. The ACC of any one or combination of items 85-86, wherein the first monomer construct further comprises a first cleavable moiety (CM1) and the MM1 is coupled to the CP1 via the CM1.
88. The ACC of any one or combination of items 85, wherein the CP2 comprises the amino acid sequence that functions as a substrate for a protease, and the MM2 is coupled to the CP2 via the amino acid sequence.
89. The ACC of any one or combination of items 85-88, wherein the second monomer construct further comprises a second cleavable moiety (CM2) and the MM2 is coupled to the CP2 via the CM2.
90. The ACC of any one or combination of items 44-89, wherein the DD1 and DD2 are a pair of human IgG Fc domains.
91. The ACC of item 90, wherein the DD1 and DD2 are a pair of human IgG4 Fc domains.
92. The ACC of item 91, wherein the DD1 and DD2 are a pair of human IgG1 or IgG4 Fe domains truncated at the N-terminus to Cysteine 226 as numbered by EU numbering.
93. The ACC of item 91, wherein the human IgG4 Fe domains comprise a S228P mutation as numbered by EU numbering.
94. The ACC of any one or combination of items 44-93, wherein each of the DD1 and DD2 comprises a sequence that is at least 95% identical to SEQ ID NO: 3.
95. The ACC of any one or combination of items 44-93, wherein each of the DD1 and DD2 comprises the sequence of SEQ ID NO: 3.
96. The ACC of any one or combination of items 44-95, wherein the first and second monomer constructs are covalently bound to each other via at least one, two, three, or four disulfide bonds.
97. The ACC of item 44, wherein each of the first and second monomer construct comprises a sequence at least 85%, 90%, or 95% identical to SEQ ID NO: 523.
98. The ACC of item 44, wherein each of the first and second monomer construct comprises SEQ ID NO: 523.
99. The ACC of any one or combination of items 24-98, wherein the ACC is characterized by having a reduced level of interleukin activity as compared to a control level of interleukin activity.
100. The ACC of any one or combination of items 24-99, wherein the ACC is characterized by having a reduced level of interleukin activity as compared to a wild type human IL-15.
101. The ACC of any one or combination of items 24-100, wherein the ACC is characterized by having a reduced level of IL-15 activity as compared to recombinant human IL-15, as measured by the level of SEAP (secreted embryonic alkaline phosphatase) production in IL-2/IL-15-responsive HEK293 cells.
102. The ACC of any one or combination of items 24-101, wherein the ACC is characterized by having a reduced level of IL-15 activity as compared to the activity of recombinant human IL-15.
103. The ACC of item 102, wherein the ACC is characterized by having a level of IL-15 activity that is reduced by at least 6000-fold as compared to recombinant human IL-15.
104. The ACC of any one or combination of items 24-103, wherein the ACC is characterized by having an EC50 following cleavage of the ACC by uPA protease that is at least 1000-, 5000-, or 6000-fold of the EC50 of recombinant wild type IL-15, as measured in IL-2/IL15-responsive HEK293 cells.
105. An ACC comprising a cytokine polypeptide (CP), an agonist of the CP, the isolated polypeptide of any one or combination of items 1-11, and a cleavable moiety, wherein the MM is coupled to the CP via the cleavable moiety.
106. The ACC of item 105, wherein the CP is IL-15, and the agonist is a Sushi domain.
107. The ACC of item 105 or item 106, wherein the agonist is coupled to the CP via a linker.
108. The ACC of item 107, wherein the agonist is coupled to the CP via a cleavable linker.
109. The ACC of item 107, wherein the agonist is coupled to the CP via a non-cleavable linker.
110. The ACC of any one or combination of items 105-106, wherein the agonist is non-covalently bound to the CP.
111. An ACC comprising a first monomer construct and a second monomer construct, wherein
   the first monomer construct comprises a cytokine polypeptide (CP), a first dimerization domain (DD1), a first cleavable moiety (CM1), a second cleavable moiety (CM2), and the isolated polypeptide of any one or combination of items 1-11, wherein the MM is coupled to the CP via the CM1, and the DD1 is coupled to the CP via the CM2,
   the second monomer construct comprises an agonist of the CP, a third cleavable moiety (CM3), a second dimerization domain (DD2) coupled to the agonist via the CM3, and
   the DD1 and DD2 bind each other thereby forming a dimer of the first and second monomer constructs.
112. The ACC of item 111, wherein the CP is IL-15, and the agonist is a Sushi domain comprising the sequence of SEQ ID NO: 520.
113. The ACC of any one or combination of items 111-112, comprising a linker consisting of 2 amino acids between the Sushi domain and the CM3.
114. A polynucleotide encoding the isolated polypeptide of any one or combination of items 1-13, the ACC of any one or combination of items 24-109, or a monomer construct of any one or combination of items 44-112.
115. A vector comprising the polynucleotide of item 114.
116. The vector of item 115, wherein the vector is an expression vector.
117. A host cell comprising the polynucleotide of item 114 or the vector of items 115 or 116.
118. The host cell of item 117, wherein the host cell is a mammalian cell.
119. A composition comprising the isolated polypeptide of any one or combination of items 1-13, or the ACC of any one or combination of items 24-113, or the polynucleotide of item 114.
120. The composition of item 119, wherein the composition is a pharmaceutical composition.
121. A container, vial, syringe, injector pen, or kit comprising at least one dose of the composition of items 119 or 120.
122. A method of treating a subject in need thereof comprising administering to the subject a therapeutically effective amount of the isolated polypeptide of any one or combination of items 1-23, the ACC of any one or combination of items 24-113, or the composition of item 119 or 120.
123. The method of item 122, wherein the subject has been identified or diagnosed as having a cancer.
124. The method of item 123, wherein the cancer is leukemia, lymphoma, or a solid tumor.
125. The method of item 122, wherein the subject has been identified or diagnosed as having an inflammatory or an autoimmune disease, disorder, or condition.
126. A method of producing an ACC comprising
   culturing a cell comprising the polynucleotide of item 114 or the host cell of item 117 or item 118 in a liquid culture medium to produce the ACC; and
   recovering the ACC from the cell or the liquid culture medium.
127. The method of item 126, further comprising isolating the ACC recovered from the cell or the liquid culture medium.
128. The method of item 126 or 127, further comprising formulating the isolated the ACC into a pharmaceutical composition.
129. A complex comprising a polypeptide comprising a cytokine complexed with the isolated polypeptide of any one or combination of items 1-16.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated polypeptide comprising amino acid sequence X₁LTTVX₂-linker-ASHYFE (SEQ ID NO: 515) (MM), wherein X₁ is absent or any amino acid, wherein X₂ is D, K, or R, and wherein the linker consists of 1 to 20 amino acids.

2. The isolated polypeptide of claim 1, comprising amino acid sequence ALTTVX-linker-ASHYFE (SEQ ID NO: 508) (MM), wherein X is D, K, or R, and wherein the N-terminal alanine residue is optionally substituted by any other amino acid, and the linker consists of 1 to 20 amino acids.

3. The isolated polypeptide of claim 1 or claim 2, wherein the X₂ or the X is D.

4. The isolated polypeptide of claim 1 or claim 2, wherein the X₂ or the X is K.

5. The isolated polypeptide of claim 1 or claim 2, wherein the X₂ or the X is R.

6. The isolated polypeptide of claim 1, wherein the X₁ is A.

7. The isolated polypeptide of claim 1 or claim 2, wherein the X₁ is K or the N-terminal alanine residue is substituted with K.

8. The isolated polypeptide of any preceding claim, wherein the linker consists of 4, 5, 6, 7, or 8 amino acids.

9. The isolated polypeptide of any preceding claim, wherein the linker consists of 6 amino acids.

10. The isolated polypeptide of any preceding claim, wherein the linker is selected from the group consisting of SEQ ID NOs: 2, 210-235, 245, 250, and 318-335.

11. The isolated polypeptide of any one of claims 1-7, wherein the linker is GGGGS (SEQ ID NO: 216).

12. The isolated polypeptide of claim 1 or claim 2, wherein the N-terminus, the C-terminus, or both of the isolated polypeptide is extended by 1, 2, 3, 4, 5, 6, 7, 8, 9, 10, 11, 12, 13, 14, 15, 16, 17, 18, 19, or 20 amino acids.

13. The isolated polypeptide of claim 12, wherein the amino acid sequence comprises ALTTVDGGGGSASHYFER (SEQ ID NO: 236) or ALTTVDGGGGSASHYFEK (SEQ ID NO: 237), or wherein the N-terminal alanine residue is optionally absent or substituted by any other amino acid.

14. The isolated polypeptide of claim 12, wherein the amino acid sequence comprises ALTTVKGGGGSASHYFER (SEQ ID NO: 238) or ALTTVKGGGGSASHYFEK (SEQ ID NO: 239), or wherein the N-terminal alanine residue is optionally absent or substituted by any other amino acid.

15. The isolated polypeptide of claim 12, wherein the amino acid sequence comprises ALTTVRGGGGSASHYFER (SEQ ID NO: 240) or ALTTVRGGGGSASHYFEK (SEQ ID NO: 241), or wherein the N-terminal alanine residue is optionally absent or substituted by any other amino acid.

16. The isolated polypeptide of claim 12, wherein the isolated polypeptide comprises a sequence selected from SQKLTTVDGGGGSASHYFERHLE (SEQ ID NO: 730), SQKLTTVRGGGGSASHYFERHLE (SEQ ID NO: 731), SQALTTVRGGGGSASHYFERHLE (SEQ ID NO: 732), or SQALTTVDGGGGSASHYFERHLE (SEQ ID NO: 733).

17. The isolated polypeptide of any one of claims 1-16, wherein the isolated polypeptide comprises a cytokine.

18. The isolated polypeptide of any one of claims 1-16, wherein the isolated polypeptide is disposed in a complex comprising two or more polypeptides, and wherein the complex comprises a cytokine.

19. The isolated polypeptide of claim 18, wherein the cytokine is disposed in a polypeptide that is complexed with the isolated polypeptide.

20. The isolated polypeptide of any one of claims 17-19, wherein the amino acid sequence is a masking moiety that inhibits binding of the cytokine with its receptor.

21. The isolated polypeptide of any one of claims 17-20, wherein the cytokine is a cytokine that binds IL2/IL15 receptor beta and/or IL2/IL15 receptor gamma.

22. The isolated polypeptide of any one of claims 17-20, wherein the cytokine binds to IL-15Rα.

23. The isolated polypeptide of any one of claims 17-20, wherein the cytokine binds to IL-2Rα.

24. An activatable cytokine construct (ACC) comprising a cytokine polypeptide (CP), a cleavable moiety (CM), and the isolated polypeptide of any one of claims 1-16, wherein the MM is coupled to the CP via the CM and inhibits the binding of CP to its receptor.

25. The ACC of claim 24, wherein the CP is an interleukin polypeptide.

26. The ACC of claim 25, wherein the interleukin polypeptide comprises a sequence at least 85%, 90%, or 95% identical to SEQ ID NO: 348, 129, or 130.

27. The ACC of claim 25, wherein the interleukin polypeptide comprises SEQ ID NO: 348, 129, or 130.

28. The ACC of claim 25, wherein the interleukin polypeptide comprises a sequence at least 85%, 90%, or 95% identical to SEQ ID NO: 119 or 120.

29. The ACC of claim 25, wherein the interleukin polypeptide comprises SEQ ID NO: 119 or 120.

30. The ACC of claim 25, wherein the interleukin polypeptide comprises a sequence at least 85%, 90%, or 95% identical to SEQ ID NO: 121 or 122.

31. The ACC of claim 25, wherein the interleukin polypeptide comprises SEQ ID NO: 121 or 122.

32. The ACC of claim 25, wherein the interleukin polypeptide comprises a sequence at least 85%, 90%, or 95% identical to SEQ ID NO: 123 or 124.

33. The ACC of claim 25, wherein the interleukin polypeptide comprises SEQ ID NO: 123 or 124.

34. The ACC of claim 25, wherein the interleukin polypeptide comprises a sequence at least 85%, 90%, or 95% identical to SEQ ID NO: 125 or 126.

35. The ACC of claim 25, wherein the interleukin polypeptide comprises SEQ ID NO: 125 or 126.

36. The ACC of claim 25, wherein the interleukin polypeptide comprises a sequence at least 85%, 90%, or 95% identical to SEQ ID NO: 521 or 522.

37. The ACC of claim 25, wherein the interleukin polypeptide comprises SEQ ID NO: 521 or 522.

38. The ACC of any one of claims 24-37, wherein the CM comprises no more than 8 amino acids.

39. The ACC of any one of claims 24-38, wherein the CM is cleavable by a urokinase (uPA) and/or a matrix metalloproteinase (MMP).

40. The ACC of any one of claims 24-39, further comprising a linker (L1) between the CM and the CP.

41. The ACC of any one of claims 24-39, further comprising a linker (L2) between the CM and the MM.

42. The ACC of any one of claims 24-41, further comprising a first linker (L1) between the CM and the CP and a second linker (L2) between the CM and the MM.

43. The ACC of any one of claims 24-42, further comprising a steric mask that further inhibits binding of the CP to its receptor.

44. An ACC comprising a first monomer construct and a second monomer construct, wherein
the first monomer construct comprises a first cytokine polypeptide (CP1), a first cleavable moiety (CM1), a first dimerization domain (DD1) coupled to the CP1 via the CM1, and a first masking moiety (MM1),
the second monomer construct comprises a second cytokine polypeptide (CP2), a second cleavable moiety (CM2), a second dimerization domain (DD2) coupled to the CP2 via the CM2, and a second masking moiety (MM2),
the DD1 and DD2 bind each other thereby forming a dimer of the first and second monomer constructs, and
the MM1 and/or the MM2 comprises the isolated polypeptide of any one of claims 1-11.

45. The ACC of claim 44, wherein the first monomer construct comprises a third cleavable moiety (CM3) and the MM1 is coupled to the CP1 via the CM3.

46. The ACC of any one of claims 44-45, wherein the MM1 is coupled to the CP1 via the DD1 and the CM1.

47. The ACC of any one of claims 44-46, wherein the second monomer construct comprises a fourth cleavable moiety (CM4) and the MM2 is coupled to the CP2 via the CM4.

48. The ACC of any one of claims 44-47, wherein the MM2 is coupled to the CP2 via the DD2 and the CM2.

49. The ACC of any one of claims 44-48, each of the first monomer construct and the second monomer construct comprises a Linking Region comprising no more than 18 amino acids.

50. The ACC of any one of claims 44-49, wherein
the CP1 and the CM1 directly abut each other, and/or
the CM1 and the DD1 directly abut each other.

51. The ACC of any one of claims 44-50, wherein
the CP2 and the CM2 directly abut each other, and/or
the CM2 and the DD2 directly abut each other.

52. The ACC of any one of claims 44-51, wherein the CP1 comprises a sequence at least 85%, 90%, or 95% identical to SEQ ID NO: 348, 129, or 130.

53. The ACC of any one of claims 44-51, wherein the CP2 comprises a sequence at least 85%, 90%, or 95% identical to SEQ ID NO: 348, 129, or 130.

54. The ACC of any one of claims 44-51, wherein the CP1 comprises SEQ ID NO: 348, 129, or 130.

55. The ACC of any one of claims 44-51, wherein the CP2 comprises SEQ ID NO: 348, 129, or 130.

56. The ACC of any one of claims 44-51, wherein the CP1 comprises a sequence at least 85%, 90%, or 95% identical to SEQ ID NO: 119 or 120.

57. The ACC of any one of claims 44-51, wherein the CP2 comprises a sequence at least 85%, 90%, or 95% identical to SEQ ID NO: 119 or 120.

58. The ACC of any one of claims 44-51, wherein the CP1 comprises SEQ ID NO: 119 or 120.

59. The ACC of any one of claims 44-51, wherein the CP2 comprises SEQ ID NO: 119 or 120.

60. The ACC of any one of claims 44-51, wherein the CP1 comprises a sequence at least 85%, 90%, or 95% identical to SEQ ID NO: 121 or 122.

61. The ACC of any one of claims 44-51, wherein the CP2 comprises a sequence at least 85%, 90%, or 95% identical to SEQ ID NO: 121 or 122.

62. The ACC of any one of claims 44-51, wherein the CP1 comprises SEQ ID NO: 121 or 122.

63. The ACC of any one of claims 44-51, wherein the CP2 comprises SEQ ID NO: 121 or 122.

64. The ACC of any one of claims 44-51, wherein the CP1 comprises a sequence at least 85%, 90%, or 95% identical to SEQ ID NO: 123 or 124.

65. The ACC of any one of claims 44-51, wherein the CP2 comprises a sequence at least 85%, 90%, or 95% identical to SEQ ID NO: 123 or 124.

66. The ACC of any one of claims 44-51, wherein the CP1 comprises SEQ ID NO: 123 or 124.

67. The ACC of any one of claims 44-51, wherein the CP2 comprises SEQ ID NO: 123 or 124.

68. The ACC of any one of claims 44-51, wherein the CP1 comprises a sequence at least 85%, 90%, or 95% identical to SEQ ID NO: 125 or 126.

69. The ACC of any one of claims 44-51, wherein the CP2 comprises a sequence at least 85%, 90%, or 95% identical to SEQ ID NO: 125 or 126.

70. The ACC of any one of claims 44-51, wherein the CP1 comprises SEQ ID NO: 125 or 126.

71. The ACC of any one of claims 44-51, wherein the CP2 comprises SEQ ID NO: 125 or 126.

72. The ACC of any one of claims 44-51, wherein the CP1 comprises a sequence at least 85%, 90%, or 95% identical to SEQ ID NO: 521 or 522.

73. The ACC of any one of claims 44-51, wherein the CP2 comprises a sequence at least 85%, 90%, or 95% identical to SEQ ID NO: 521 or 522.

74. The ACC of any one of claims 44-51, wherein the CP1 comprises SEQ ID NO: 521 or 522.

75. The ACC of any one of claims 44-51, wherein the CP2 comprises SEQ ID NO: 521 or 522.

76. The ACC of any one of claims 44-75, wherein the first monomer construct and the second monomer construct are identical.

77. The ACC of any one of claims 44-76, wherein the first monomer construct and the second monomer construct have 95% sequence homology.

78. The ACC of any one of claims 44-77, further comprising a third monomer comprising a Sushi domain comprising of the sequence of SEQ ID NO: 520, wherein the third monomer is non-covalently or covalently bound to the ACC.

79. The ACC of any one of claims 44-78, further comprising a fourth monomer comprising a Sushi domain comprising of the sequence of SEQ ID NO: 520, wherein the fourth monomer is non-covalently or covalently bound to the ACC.

80. The ACC of claims 78 or 79, wherein the third and/or fourth monomer further comprises a tag.

81. An ACC comprising a first monomer construct and a second monomer construct, wherein
the first monomer construct comprises a first cytokine polypeptide (CP1), a first dimerization domain (DD1), and a first masking moiety (MM1),
the second monomer construct comprises a second cytokine polypeptide (CP2), a first cleavable moiety (CM1), a second dimerization domain (DD2) coupled to the CP2 via the CM1, and a second masking moiety (MM2),
the MM1 and/or the MM2 is the isolated polypeptide of any one of claims 1-11, and
the DD1 and DD2 bind each other thereby forming a dimer of the first and second monomer constructs.

82. The ACC of claim 81, wherein the first monomer construct further comprises a second cleavable moiety (CM2) and the MM1 is coupled to the CP1 via the CM2.

83. The ACC of claim 81 or 82, wherein the MM2 is coupled to the CP2 via the DD2 and the CM1.

84. The ACC of any one of claims 81-83, wherein the second monomer construct further comprises a third cleavable moiety (CM3), wherein the MM2 is coupled to the CP2 via the CM3.

85. An ACC comprising a first monomer construct and a second monomer construct, wherein
the first monomer construct comprises a first cytokine polypeptide (CP1), a first dimerization domain (DD1), and a first masking moiety (MM1),
the second monomer construct comprises a second cytokine polypeptide (CP2), a second dimerization domain (DD2), and a second masking moiety (MM2),
the CP1 and/or the CP2 comprises an amino acid sequence that functions as a substrate for a protease, and the DD1 and/or DD2 is coupled to the CP1 and/or CP2 via the amino acid sequence,
the MM1 and/or the MM2 is the isolated polypeptide of any one of claims 1-11, and
the DD1 and DD2 bind each other thereby forming a dimer of the first and second monomer constructs.

86. The ACC of claim 85, wherein the CP1 comprises the amino acid sequence that functions as a substrate for a protease, and the MM1 is coupled to the CP1 via the amino acid sequence.

87. The ACC of any one of claims 85-86, wherein the first monomer construct further comprises a first cleavable moiety (CM1) and the MM1 is coupled to the CP1 via the CM1.

88. The ACC of any one of claim 85, wherein the CP2 comprises the amino acid sequence that functions as a substrate for a protease, and the MM2 is coupled to the CP2 via the amino acid sequence.

89. The ACC of any one of claims 85-88, wherein the second monomer construct further comprises a second cleavable moiety (CM2) and the MM2 is coupled to the CP2 via the CM2.

90. The ACC of any one of claims 44-89, wherein the DD1 and DD2 are a pair of human IgG Fc domains.

91. The ACC of claim 90, wherein the DD1 and DD2 are a pair of human IgG4 Fc domains.

92. The ACC of claim 91, wherein the DD1 and DD2 are a pair of human IgG1 or IgG4 Fc domains truncated at the N-terminus to Cysteine 226 as numbered by EU numbering.

93. The ACC of claim 91, wherein the human IgG4 Fc domains comprise a S228P mutation as numbered by EU numbering.

94. The ACC of any one of claims 44-93, wherein each of the DD1 and DD2 comprises a sequence that is at least 95% identical to SEQ ID NO: 3.

95. The ACC of any one of claims 44-93, wherein each of the DD1 and DD2 comprises the sequence of SEQ ID NO: 3.

96. The ACC of any one of claims 44-95, wherein the first and second monomer constructs are covalently bound to each other via at least one, two, three, or four disulfide bonds.

97. The ACC of claim 44, wherein each of the first and second monomer construct comprises a sequence at least 85%, 90%, or 95% identical to SEQ ID NO: 523.

98. The ACC of claim 44, wherein each of the first and second monomer construct comprises SEQ ID NO: 523.

99. The ACC of any one of claims 24-98, wherein the ACC is characterized by having a reduced level of interleukin activity as compared to a control level of interleukin activity.

100. The ACC any one of claims 24-99, wherein the ACC is characterized by having a reduced level of interleukin activity as compared to a wild type human IL-15.

101. The ACC of any one of claims 24-100, wherein the ACC is characterized by having a reduced level of IL-15 activity as compared to recombinant human IL-15, as measured by the level of SEAP (secreted embryonic alkaline phosphatase) production in IL-2/IL-15-responsive HEK293 cells.

102. The ACC of any one of claims 24-101, wherein the ACC is characterized by having a reduced level of IL-15 activity as compared to the activity of recombinant human IL-15.

103. The ACC of claim 102, wherein the ACC is characterized by having a level of IL-15 activity that is reduced by at least 6000-fold as compared to recombinant human IL-15.

104. The ACC of any one of claims 24-103, wherein the ACC is characterized by having an EC50 following cleavage of the ACC by uPA protease that is at least 1000-, 5000-, or 6000-fold of the EC50 of recombinant wild type IL-15, as measured in IL-2/IL15-responsive HEK293 cells.

105. An ACC comprising a cytokine polypeptide (CP), an agonist of the CP, the isolated polypeptide of any one of claims 1-11, and a cleavable moiety, wherein the MM is coupled to the CP via the cleavable moiety.

106. The ACC of claim 105, wherein the CP is IL-15, and the agonist is a Sushi domain.

107. The ACC of claim 105 or claim 106, wherein the agonist is coupled to the CP via a linker.

108. The ACC of claim 107, wherein the agonist is coupled to the CP via a cleavable linker.

109. The ACC of claim 107, wherein the agonist is coupled to the CP via a non-cleavable linker.

110. The ACC of claim 105 or claim 106, wherein the agonist is non-covalently bound to the CP.

111. An ACC comprising a first monomer construct and a second monomer construct, wherein
the first monomer construct comprises a cytokine polypeptide (CP), a first dimerization domain (DD1), a first cleavable moiety (CM1), a second cleavable moiety (CM2), and the isolated polypeptide of any one of claims 1-11, wherein the MM is coupled to the CP via the CM1, and the DD1 is coupled to the CP via the CM2,
the second monomer construct comprises an agonist of the CP, a third cleavable moiety (CM3), a second dimerization domain (DD2) coupled to the agonist via the CM3, and
the DD1 and DD2 bind each other thereby forming a dimer of the first and second monomer constructs.

112. The ACC of claim 111, wherein the CP is IL-15, and the agonist is a Sushi domain comprising the sequence of SEQ ID NO: 520.

113. The ACC of claim 111 or 112, comprising a linker consisting of 2 amino acids between the Sushi domain and the CM3.

114. A polynucleotide encoding the isolated polypeptide of any one of claims 1-13, the ACC of any one of claims 24-109, or a monomer construct of any one of claims 44-112.

115. A vector comprising the polynucleotide of claim 114.

116. The vector of claim 115, wherein the vector is an expression vector.

117. A host cell comprising the polynucleotide of claim 114 or the vector of claim 115 or 116.

118. The host cell of claim 117, wherein the host cell is a mammalian cell.

119. A composition comprising the isolated polypeptide of any one of claim 1-13, or the ACC of any one of claims 24-113, or the polynucleotide of claim 114.

120. The composition of claim 119, wherein the composition is a pharmaceutical composition.

121. A container, vial, syringe, injector pen, or kit comprising at least one dose of the composition of claim 119 or 120.

122. A method of treating a subject in need thereof comprising administering to the subject a therapeutically effective amount of the isolated polypeptide of any one of claim 1-23, the ACC of any one of claims 24-113, or the composition of claim 119 or 120.

123. The method of claim 122, wherein the subject has been identified or diagnosed as having a cancer.

124. The method of claim 123, wherein the cancer is leukemia, lymphoma, or a solid tumor.

125. The method of claim 122, wherein the subject has been identified or diagnosed as having an inflammatory or an autoimmune disease, disorder, or condition.

126. A method of producing an ACC comprising
culturing a cell comprising the polynucleotide of claim 114 or the host cell of claim 117 or claim 118 in a liquid culture medium to produce the ACC; and
recovering the ACC from the cell or the liquid culture medium.

127. The method of claim 126, further comprising isolating the ACC recovered from the cell or the liquid culture medium.

128. The method of claim 126 or 127, further comprising formulating the isolated the ACC into a pharmaceutical composition.

129. A complex comprising a polypeptide comprising a cytokine complexed with the isolated polypeptide of any one of claims 1-16.


SEQUENCE LISTING (734 sequences)

SEQ ID NO: 1 - skipped

SEQ ID NO: 2 - protein, 4 aa, synthetic construct
GGGS

SEQ ID NO: 3 - protein, 219 aa, Homo sapiens
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 4 - protein, 226 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 5 - protein, 10 aa, synthetic construct
AANALAHGLF

SEQ ID NO: 6 - protein, 4 aa, synthetic construct
AANL

SEQ ID NO: 7 - protein, 10 aa, synthetic construct
AANLGSGGSS

SEQ ID NO: 8 - protein, 5 aa, synthetic construct
AAPRS

SEQ ID NO: 9 - protein, 6 aa, synthetic construct
AAPRSF

SEQ ID NO: 10 - protein, 8 aa, synthetic construct
AARGPAIH

SEQ ID NO: 11 - protein, 8 aa, synthetic construct
AAYHLVSQ

SEQ ID NO: 12 - protein, 10 aa, synthetic construct
AFPDMRSVRS

SEQ ID NO: 13 - protein, 7 aa, synthetic construct
AFQALRM

SEQ ID NO: 14 - protein, 6 aa, synthetic construct
AFRHLR

SEQ ID NO: 15 - protein, 8 aa, synthetic construct
AGLGISST

SEQ ID NO: 16 - protein, 8 aa, synthetic construct
AGLGVVER

SEQ ID NO: 17 - protein, 4 aa, synthetic construct
AGPR

SEQ ID NO: 18 - protein, 4 aa, synthetic construct
AHGL

SEQ ID NO: 19 - protein, 5 aa, synthetic construct
AHGLF

SEQ ID NO: 20 - protein, 7 aa, synthetic construct
AHQALRM

SEQ ID NO: 21 - protein, 10 aa, synthetic construct
AIPRVRLFDV

SEQ ID NO: 22 - protein, 5 aa, synthetic construct
ALAHG

SEQ ID NO: 23 - protein, 6 aa, synthetic construct
ALAHGL

SEQ ID NO: 24 - protein, 7 aa, synthetic construct
ALAHGLF

SEQ ID NO: 25 - protein, 12 aa, synthetic construct
ALAHGLFAPRSF

SEQ ID NO: 26 - protein, 13 aa, synthetic construct
ALAHGLFSGRSAN

SEQ ID NO: 27 - protein, 12 aa, synthetic construct
ALAHGLPTFVHL

SEQ ID NO: 28 - protein, 8 aa, synthetic construct
ALGLLRLP

SEQ ID NO: 29 - protein, 10 aa, synthetic construct
ALPSVKMVSE

SEQ ID NO: 30 - protein, 5 aa, synthetic construct
ALRAP

SEQ ID NO: 31 - protein, 7 aa, synthetic construct
ANQALRM

SEQ ID NO: 32 - protein, 8 aa, synthetic construct
ANQALRMA

SEQ ID NO: 33 - protein, 10 aa, synthetic construct
APPLVKSMVV

SEQ ID NO: 34 - protein, 10 aa, synthetic construct
APPSFKLVNA

SEQ ID NO: 35 - protein, 4 aa, synthetic construct
APRS

SEQ ID NO: 36 - protein, 11 aa, synthetic construct
APRSALAHGLF

SEQ ID NO: 37 - protein, 5 aa, synthetic construct
APRSF

SEQ ID NO: 38 - protein, 8 aa, synthetic construct
AQFVLTEG

SEQ ID NO: 39 - protein, 8 aa, synthetic construct
AQNLLGMV

SEQ ID NO: 40 - protein, 4 aa, synthetic construct
ARGP

SEQ ID NO: 41 - protein, 5 aa, synthetic construct
ARGPS

SEQ ID NO: 42 - protein, 6 aa, synthetic construct
ARGPSF

SEQ ID NO: 43 - protein, 7 aa, synthetic construct
ARGPSFK

SEQ ID NO: 44 - protein, 8 aa, synthetic construct
ASGLLRFP

SEQ ID NO: 45 - protein, 10 aa, synthetic construct
ASPTMKTVGL

SEQ ID NO: 46 - protein, 8 aa, synthetic construct
AVGLLAPP

SEQ ID NO: 47 - protein, 18 aa, synthetic construct
AVGLLAPPGGLSGRSANI

SEQ ID NO: 48 - protein, 18 aa, synthetic construct
AVGLLAPPGGLSGRSANP

SEQ ID NO: 49 - protein, 18 aa, synthetic construct
AVGLLAPPGGLSGRSDDH

SEQ ID NO: 50 - protein, 18 aa, synthetic construct
AVGLLAPPGGLSGRSDIH

SEQ ID NO: 51 - protein, 18 aa, synthetic construct
AVGLLAPPGGLSGRSDNH

SEQ ID NO: 52 - protein, 18 aa, synthetic construct
AVGLLAPPGGLSGRSDNI

SEQ ID NO: 53 - protein, 18 aa, synthetic construct
AVGLLAPPGGLSGRSDNP

SEQ ID NO: 54 - protein, 18 aa, synthetic construct
AVGLLAPPGGLSGRSDQH

SEQ ID NO: 55 - protein, 18 aa, synthetic construct
AVGLLAPPGGLSGRSDTH

SEQ ID NO: 56 - protein, 18 aa, synthetic construct
AVGLLAPPGGLSGRSDYH

SEQ ID NO: 57 - protein, 17 aa, synthetic construct
AVGLLAPPGGLSGRSNI

SEQ ID NO: 58 - protein, 18 aa, synthetic construct
AVGLLAPPGGLSGRSNIG

SEQ ID NO: 59 - protein, 19 aa, synthetic construct
AVGLLAPPGGLSGRSNIGS

SEQ ID NO: 60 - protein, 22 aa, synthetic construct
AVGLLAPPGGTSTSGRSANPRG

SEQ ID NO: 61 - protein, 17 aa, synthetic construct
AVGLLAPPSGRSANPRG

SEQ ID NO: 62 - protein, 18 aa, synthetic construct
AVGLLAPPTSGRSANPRG

SEQ ID NO: 63 - protein, 10 aa, synthetic construct
AVPKVRVVPE

SEQ ID NO: 64 - protein, 7 aa, synthetic construct
CGPPLGR

SEQ ID NO: 65 - protein, 7 aa, synthetic construct
CSPPLGR

SEQ ID NO: 66 - protein, 10 aa, synthetic construct
DEVDGSGGSS

SEQ ID NO: 67 - skipped

SEQ ID NO: 68 - protein, 8 aa, synthetic construct
DISHWRRS

SEQ ID NO: 69 - protein, 7 aa, synthetic construct
DLAHPLL

SEQ ID NO: 70 - protein, 10 aa, synthetic construct
DLPLVKSLPS

SEQ ID NO: 71 - skipped

SEQ ID NO: 72 - protein, 12 aa, synthetic construct
DRLSGRSANHKK

SEQ ID NO: 73 - protein, 12 aa, synthetic construct
DRLSGRSDNHKK

SEQ ID NO: 74 - protein, 10 aa, synthetic construct
DRPEMKSLSG

SEQ ID NO: 75 - protein, 10 aa, synthetic construct
DRPKVKTMDF

SEQ ID NO: 76 - protein, 8 aa, synthetic construct
DVAQFVLT

SEQ ID NO: 77 - protein, 10 aa, synthetic construct
DVPPMKTLRP

SEQ ID NO: 78 - protein, 8 aa, synthetic construct
DWLYWMGI

SEQ ID NO: 79 - protein, 8 aa, synthetic construct
DWLYWMSI

SEQ ID NO: 80 - protein, 8 aa, synthetic construct
DWLYWPGI

SEQ ID NO: 81 - protein, 8 aa, synthetic construct
DWLYWPSI

SEQ ID NO: 82 - protein, 10 aa, synthetic construct
EAPKVKALPK

SEQ ID NO: 83 - protein, 10 aa, synthetic construct
EHPRVKVVSE

SEQ ID NO: 84 - protein, 10 aa, synthetic construct
EKPRMKLFQG

SEQ ID NO: 85 - protein, 8 aa, synthetic construct
EPQALAMS

SEQ ID NO: 86 - protein, 10 aa, synthetic construct
EQPEVKMVKG

SEQ ID NO: 87 - protein, 10 aa, synthetic construct
ERPGVKSLVL

SEQ ID NO: 88 - protein, 8 aa, synthetic construct
ESLPVVAV

SEQ ID NO: 89 - protein, 10 aa, synthetic construct
ESPVMKSMAL

SEQ ID NO: 90 - protein, 5 aa, synthetic construct
ESRRW

SEQ ID NO: 91 - protein, 6 aa, synthetic construct
ESRRWM

SEQ ID NO: 92 - protein, 7 aa, synthetic construct
ESRRWMP

SEQ ID NO: 93 - protein, 10 aa, synthetic construct
ETPSVKTMGR

SEQ ID NO: 94 - protein, 12 aa, synthetic construct
ETPSVKTMGRSS

SEQ ID NO: 95 - protein, 10 aa, synthetic construct
FPRPLGITGL

SEQ ID NO: 96 - protein, 8 aa, synthetic construct
FRLLDWQW

SEQ ID NO: 97 - protein, 8 aa, synthetic construct
GCGPPLGR

SEQ ID NO: 98 - protein, 8 aa, synthetic construct
GCSPPLGR

SEQ ID NO: 99 - protein, 10 aa, synthetic construct
GFPHMKTFQH

SEQ ID NO: 100 - protein, 12 aa, synthetic construct
GFPHMKTFQHSS

SEQ ID NO: 101 - protein, 8 aa, synthetic construct
GGGPPLGR

SEQ ID NO: 102 - protein, 7 aa, synthetic construct
GGPPLGR

SEQ ID NO: 103 - protein, 10 aa, synthetic construct
GGQPSGMWGW

SEQ ID NO: 104 - protein, 7 aa, synthetic construct
GGSIDGR

SEQ ID NO: 105 - protein, 8 aa, synthetic construct
GGSPPLGR

SEQ ID NO: 106 - protein, 8 aa, synthetic construct
GGWHTGRN

SEQ ID NO: 107 - protein, 5 aa, synthetic construct
GIAGQ

SEQ ID NO: 108 - protein, 10 aa, synthetic construct
GLGTPRGLFA

SEQ ID NO: 109 - protein, 6 aa, synthetic construct
GLPTFV

SEQ ID NO: 110 - protein, 7 aa, synthetic construct
GLPTFVH

SEQ ID NO: 111 - protein, 8 aa, synthetic construct
GLPTFVHL

SEQ ID NO: 112 - protein, 12 aa, synthetic construct
GLPTFVHLPRQV

SEQ ID NO: 113 - protein, 12 aa, synthetic construct
GLSGRSDNHGSS

SEQ ID NO: 114 - protein, 8 aa, synthetic construct
GPEGLRVG

SEQ ID NO: 115 - protein, 8 aa, synthetic construct
GPLGIAGI

SEQ ID NO: 116 - protein, 12 aa, synthetic construct
GPLNGRSDNHKA

SEQ ID NO: 117 - protein, 12 aa, synthetic construct
GPLNGRSDNHKK

SEQ ID NO: 118 - protein, 12 aa, synthetic construct
GPLNGRSDNHKR

SEQ ID NO: 119 - protein, 150 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIS

SEQ ID NO: 120 - protein, 169 aa, Mus musculus
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 121 - protein, 153 aa, Homo sapiens [sequence omitted]

SEQ ID NO: 122 - protein, 140 aa, Mus musculus
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 123 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 124 - protein, 154 aa, Mus musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 125 - protein, 144 aa, Homo sapiens
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 126 - protein, 144 aa, Mus musculus
MLVTYILASVLLFSSVLGQRCSTTWGIRDTNYLIENLKDDPPSKCSCSGNVTSCLCLSVPTDDCTTPCYREGLLQLTNATQKSRLLPVFHRVKRIVEVLKNITCPSFSCEKPCNQTMAGNTLSFLKSLLGTFQKTEMQRQKSRP

SEQ ID NO: 127-128 - skipped (2 entries)

SEQ ID NO: 129 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 130 - protein, 162 aa, Mus musculus
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 131 - protein, 12 aa, synthetic construct
GPLNGRSDNHQA

SEQ ID NO: 132 - protein, 12 aa, synthetic construct
GPLNGRSDNHQK

SEQ ID NO: 133 - protein, 12 aa, synthetic construct
GPLNGRSDNHQR

SEQ ID NO: 134 - protein, 12 aa, synthetic construct
GPLNGRSDNHRA

SEQ ID NO: 135 - protein, 12 aa, synthetic construct
GPLNGRSDNHRK

SEQ ID NO: 136 - protein, 12 aa, synthetic construct
GPLNGRSDNHRR

SEQ ID NO: 137 - protein, 12 aa, synthetic construct
GPLSGRSDNHKA

SEQ ID NO: 138 - protein, 12 aa, synthetic construct
GPLSGRSDNHKK

SEQ ID NO: 139 - protein, 12 aa, synthetic construct
GPLSGRSDNHKR

SEQ ID NO: 140 - protein, 12 aa, synthetic construct
GPLSGRSDNHQA

SEQ ID NO: 141 - protein, 12 aa, synthetic construct
GPLSGRSDNHQK

SEQ ID NO: 142 - protein, 12 aa, synthetic construct
GPLSGRSDNHQR

SEQ ID NO: 143 - protein, 12 aa, synthetic construct
GPLSGRSDNHRA

SEQ ID NO: 144 - protein, 12 aa, synthetic construct
GPLSGRSDNHRK

SEQ ID NO: 145 - protein, 12 aa, synthetic construct
GPLSGRSDNHRR

SEQ ID NO: 146 - protein, 6 aa, synthetic construct
GPPLGR

SEQ ID NO: 147 - protein, 8 aa, synthetic construct
GPQGIAGQ

SEQ ID NO: 148 - protein, 8 aa, synthetic construct
GPQGLLGA

SEQ ID NO: 149 - protein, 6 aa, synthetic construct
GPRSFG

SEQ ID NO: 150 - protein, 7 aa, synthetic construct
GPRSFGL

SEQ ID NO: 151 - protein, 8 aa, synthetic construct
GPSHLVLT

SEQ ID NO: 152 - protein, 4 aa, synthetic construct
GPTN

SEQ ID NO: 153 - protein, 11 aa, synthetic construct
GPTNALAHGLF

SEQ ID NO: 154 - protein, 5 aa, synthetic construct
GRSML

SEQ ID NO: 155 - protein, 6 aa, synthetic construct
GRSMLL

SEQ ID NO: 156 - protein, 7 aa, synthetic construct
GRSMLLG

SEQ ID NO: 157 - protein, 8 aa, synthetic construct
GRSMLLGG

SEQ ID NO: 158 - protein, 8 aa, synthetic construct
GRSMLLGP

SEQ ID NO: 159 - protein, 8 aa, synthetic construct
GRSMLLGS

SEQ ID NO: 160 - protein, 7 aa, synthetic construct
GRSMLLP

SEQ ID NO: 161 - protein, 8 aa, synthetic construct
GRSMLLPG

SEQ ID NO: 162 - protein, 8 aa, synthetic construct
GRSMLLPP

SEQ ID NO: 163 - protein, 8 aa, synthetic construct
GRSMLLPS

SEQ ID NO: 164 - protein, 7 aa, synthetic construct
GRSMLLS

SEQ ID NO: 165 - protein, 8 aa, synthetic construct
GRSMLLSG

SEQ ID NO: 166 - protein, 8 aa, synthetic construct
GRSMLLSP

SEQ ID NO: 167 - protein, 8 aa, synthetic construct
GRSMLLSS

SEQ ID NO: 168 - protein, 6 aa, synthetic construct
GRSMLM

SEQ ID NO: 169 - protein, 7 aa, synthetic construct
GRSMLMG

SEQ ID NO: 170 - protein, 8 aa, synthetic construct
GRSMLMGG

SEQ ID NO: 171 - protein, 8 aa, synthetic construct
GRSMLMGP

SEQ ID NO: 172 - protein, 8 aa, synthetic construct
GRSMLMGS

SEQ ID NO: 173 - protein, 7 aa, synthetic construct
GRSMLMP

SEQ ID NO: 174 - protein, 8 aa, synthetic construct
GRSMLMPG

SEQ ID NO: 175 - protein, 8 aa, synthetic construct
GRSMLMPP

SEQ ID NO: 176 - protein, 8 aa, synthetic construct
GRSMLMPS

SEQ ID NO: 177 - protein, 7 aa, synthetic construct
GRSMLMS

SEQ ID NO: 178 - protein, 8 aa, synthetic construct
GRSMLMSG

SEQ ID NO: 179 - protein, 8 aa, synthetic construct
GRSMLMSP

SEQ ID NO: 180 - protein, 8 aa, synthetic construct
GRSMLMSS

SEQ ID NO: 181 - protein, 8 aa, synthetic construct
GSGPPLGR

SEQ ID NO: 182 - protein, 7 aa, synthetic construct
GSPPLGR

SEQ ID NO: 183 - protein, 8 aa, synthetic construct
GSSPPLGR

SEQ ID NO: 184 - protein, 12 aa, synthetic construct
GTGRGPSWVGSS

SEQ ID NO: 185 - protein, 4 aa, synthetic construct
PTNL

SEQ ID NO: 186 - protein, 10 aa, synthetic construct
PTNLGSGGSS

SEQ ID NO: 187 - protein, 8 aa, synthetic construct
PVGYTSSL

SEQ ID NO: 188 - protein, 10 aa, synthetic construct
PVPRLKLIKD

SEQ ID NO: 189 - protein, 8 aa, synthetic construct
PVQPIGPQ

SEQ ID NO: 190 - protein, 8 aa, synthetic construct
QALAMSAI

SEQ ID NO: 191 - protein, 7 aa, synthetic construct
QFQALRM

SEQ ID NO: 192 - protein, 10 aa, synthetic construct
QGPMFKSLWD

SEQ ID NO: 193 - protein, 8 aa, synthetic construct
QGRAITFI

SEQ ID NO: 194 - protein, 7 aa, synthetic construct
QHQALRM

SEQ ID NO: 195 - protein, 8 aa, synthetic construct
QNQALRIA

SEQ ID NO: 196 - protein, 7 aa, synthetic construct
QNQALRM

SEQ ID NO: 197 - protein, 8 aa, synthetic construct
QNQALRMA

SEQ ID NO: 198 - protein, 22 aa, synthetic construct
QNQALRMAGGSGGSLSGRSDNH

SEQ ID NO: 199 - protein, 22 aa, synthetic construct
QNQALRMAGGSGGSLSGRSGNH

SEQ ID NO: 200 - protein, 6 aa, synthetic construct
QSRRVP

SEQ ID NO: 201 - protein, 7 aa, synthetic construct
QSRRVPL

SEQ ID NO: 202 - protein, 7 aa, synthetic construct
QSRRVPV

SEQ ID NO: 203 - protein, 6 aa, synthetic construct
QTRRVP

SEQ ID NO: 204 - protein, 7 aa, synthetic construct
QTRRVPL

SEQ ID NO: 205 - protein, 7 aa, synthetic construct
QTRRVPV

SEQ ID NO: 206 - protein, 8 aa, synthetic construct
QYIVSRSA

SEQ ID NO: 207 - protein, 6 aa, synthetic construct
RALRAP

SEQ ID NO: 208 - protein, 10 aa, synthetic construct
REPFMKSLPW

SEQ ID NO: 209 - protein, 6 aa, synthetic construct
RFPLKV

SEQ ID NO: 210 - protein, 11 aa, synthetic construct
GSSGGSGGSGG

SEQ ID NO: 211 - protein, 8 aa, synthetic construct
GGGSGGGS

SEQ ID NO: 212 - protein, 12 aa, synthetic construct
GGGSGGGSGGGS

SEQ ID NO: 213 - protein, 15 aa, synthetic construct
GGGGSGGGGSGGGGS

SEQ ID NO: 214 - protein, 25 aa, synthetic construct
GGGGSGGGGSGGGGSGGGGSGGGGS

SEQ ID NO: 215 - protein, 10 aa, synthetic construct
GGGGSGGGGS

SEQ ID NO: 216 - protein, 5 aa, synthetic construct
GGGGS

SEQ ID NO: 217 - protein, 7 aa, synthetic construct
GGGGSGS

SEQ ID NO: 218 - protein, 17 aa, synthetic construct
GGGGSGGGGSGGGGSGS

SEQ ID NO: 219 - protein, 12 aa, synthetic construct
GGSLDPKGGGGS

SEQ ID NO: 220 - protein, 20 aa, synthetic construct
PKSCDKTHTCPPCPAPELLG

SEQ ID NO: 221 - protein, 17 aa, synthetic construct
SKYGPPCPPCPAPEFLG

SEQ ID NO: 222 - protein, 12 aa, synthetic construct
GKSSGSGSESKS

SEQ ID NO: 223 - protein, 14 aa, synthetic construct
GSTSGSGKSSEGKG

SEQ ID NO: 224 - protein, 18 aa, synthetic construct
GSTSGSGKSSEGSGSTKG

SEQ ID NO: 225 - protein, 18 aa, synthetic construct
GSTSGSGKPGSGEGSTKG

SEQ ID NO: 226 - protein, 16 aa, synthetic construct
GSTSGSGKPGSSEGST

SEQ ID NO: 227 - protein, 5 aa, synthetic construct
GSGGS

SEQ ID NO: 228 - protein, 4 aa, synthetic construct
GGGS

SEQ ID NO: 229 - protein, 4 aa, synthetic construct
GGSG

SEQ ID NO: 230 - protein, 5 aa, synthetic construct
GGSGG

SEQ ID NO: 231 - protein, 5 aa, synthetic construct
GSGSG

SEQ ID NO: 232 - protein, 5 aa, synthetic construct
GSGGG

SEQ ID NO: 233 - protein, 5 aa, synthetic construct
GGGSG

SEQ ID NO: 234 - protein, 5 aa, synthetic construct
GSSSG

SEQ ID NO: 235 - protein, 20 aa, synthetic construct
GGGGSGGGGSGGGGSGGGGS

SEQ ID NO: 236 - protein, 18 aa, synthetic construct
ALTTVDGGGGSASHYFER

SEQ ID NO: 237 - protein, 18 aa, synthetic construct
ALTTVDGGGGSASHYFEK

SEQ ID NO: 238 - protein, 18 aa, synthetic construct
ALTTVKGGGGSASHYFER

SEQ ID NO: 239 - protein, 18 aa, synthetic construct
ALTTVKGGGGSASHYFEK

SEQ ID NO: 240 - protein, 18 aa, synthetic construct
ALTTVRGGGGSAAHYFER

SEQ ID NO: 241 - protein, 18 aa, synthetic construct
ALTTVRGGGGSASHYFEK

SEQ ID NO: 242 - protein, 33 aa, synthetic construct
  VARIANT 8..26: note = X is any amino acid or absent
ALTTVDXXXXXXXXXXXXXXXXXXXXASHYFER

SEQ ID NO: 243 - protein, 33 aa, synthetic construct
  VARIANT 8..26: note = X is any amino acid or absent
ALTTVDXXXXXXXXXXXXXXXXXXXXASHYFEK

SEQ ID NO: 244 - protein, 33 aa, synthetic construct
  VARIANT 8..26: note = X is any amino acid or absent
ALTTVKXXXXXXXXXXXXXXXXXXXXASHYFER

SEQ ID NO: 245 - protein, 8 aa, synthetic construct
GPQGTAGQ

SEQ ID NO: 246 - protein, 33 aa, synthetic construct
  VARIANT 8..26: note = X is any amino acid or absent
ALTTVKXXXXXXXXXXXXXXXXXXXXASHYFEK

SEQ ID NO: 247 - protein, 33 aa, synthetic construct
  VARIANT 8..26: note = X is any amino acid or absent
ALTTVRXXXXXXXXXXXXXXXXXXXXASHYFER

SEQ ID NO: 248 - protein, 33 aa, synthetic construct
  VARIANT 8..26: note = X is any amino acid or absent
ALTTVRXXXXXXXXXXXXXXXXXXXXASHYFEK

SEQ ID NO: 249 - skipped

SEQ ID NO: 250 - protein, 7 aa, synthetic construct
YGAGLGW

SEQ ID NO: 251 - protein, 10 aa, synthetic construct
RFPSLKSFPL

SEQ ID NO: 252 - protein, 7 aa, synthetic construct
RFPYGVW

SEQ ID NO: 253 - protein, 8 aa, synthetic construct
RFYRNQFF

SEQ ID NO: 254 - protein, 4 aa, synthetic construct
RGPA

SEQ ID NO: 255 - protein, 8 aa, synthetic construct
RGPAFNPM

SEQ ID NO: 256 - protein, 8 aa, synthetic construct
RGPATPIM

SEQ ID NO: 257 - protein, 7 aa, synthetic construct
RGPKLYW

SEQ ID NO: 258 - protein, 6 aa, synthetic construct
RHLAKL

SEQ ID NO: 259 - protein, 8 aa, synthetic construct
RIGRSDNH

SEQ ID NO: 260 - protein, 8 aa, synthetic construct
RKMPNITV

SEQ ID NO: 261 - protein, 14 aa, synthetic construct
RKSSIIIRMRDVVL

SEQ ID NO: 262 - protein, 8 aa, synthetic construct
RKTVQHWW

SEQ ID NO: 263 - protein, 8 aa, synthetic construct
RLGRSDNN

SEQ ID NO: 264 - protein, 8 aa, synthetic construct
RMHLRSLG

SEQ ID NO: 265 - protein, 8 aa, synthetic construct
RPLARAGI

SEQ ID NO: 266 - protein, 8 aa, synthetic construct
RPLARAGL

SEQ ID NO: 267 - protein, 12 aa, synthetic construct
RPLNGRSDNHKA

SEQ ID NO: 268 - protein, 12 aa, synthetic construct
RPLNGRSDNHKK

SEQ ID NO: 269 - protein, 12 aa, synthetic construct
RPLNGRSDNHKR

SEQ ID NO: 270 - protein, 12 aa, synthetic construct
RPLNGRSDNHQA

SEQ ID NO: 271 - protein, 12 aa, synthetic construct
RPLNGRSDNHQK

SEQ ID NO: 272 - protein, 12 aa, synthetic construct
RPLNGRSDNHQR

SEQ ID NO: 273 - protein, 12 aa, synthetic construct
RPLNGRSDNHRA

SEQ ID NO: 274 - protein, 12 aa, synthetic construct
RPLNGRSDNHRK

SEQ ID NO: 275 - protein, 12 aa, synthetic construct
RPLNGRSDNHRR

SEQ ID NO: 276 - protein, 12 aa, synthetic construct
RPLSGRSDNHKA

SEQ ID NO: 277 - protein, 12 aa, synthetic construct
RPLSGRSDNHKK

SEQ ID NO: 278 - protein, 12 aa, synthetic construct
RPLSGRSDNHKR

SEQ ID NO: 279 - protein, 12 aa, synthetic construct
RPLSGRSDNHQA

SEQ ID NO: 280 - protein, 12 aa, synthetic construct
RPLSGRSDNHQK

SEQ ID NO: 281 - protein, 12 aa, synthetic construct
RPLSGRSDNHQR

SEQ ID NO: 282 - protein, 12 aa, synthetic construct
RPLSGRSDNHRA

SEQ ID NO: 283 - protein, 12 aa, synthetic construct
RPLSGRSDNHRK

SEQ ID NO: 284 - protein, 12 aa, synthetic construct
RPLSGRSDNHRR

SEQ ID NO: 285 - protein, 8 aa, synthetic construct
RPSPMWAY

SEQ ID NO: 286 - protein, 8 aa, synthetic construct
RRHDGLRA

SEQ ID NO: 287 - protein, 8 aa, synthetic construct
RRHDGLRS

SEQ ID NO: 288 - protein, 8 aa, synthetic construct
RSLVFAPI

SEQ ID NO: 289 - protein, 8 aa, synthetic construct
RSPSRLKC

SEQ ID NO: 290 - protein, 8 aa, synthetic construct
VAGRSMRP

SEQ ID NO: 291 - protein, 10 aa, synthetic construct
VAPQLKSLVP

SEQ ID NO: 292 - protein, 8 aa, synthetic construct
VAQFVLTE

SEQ ID NO: 293 - protein, 10 aa, synthetic construct
VHMPLGFLGP

SEQ ID NO: 294 - protein, 20 aa, synthetic construct
VHMPLGFLGPGGLSGRSDNH

SEQ ID NO: 295 - protein, 24 aa, synthetic construct
VHMPLGFLGPGGTSTSGRSANPRG

SEQ ID NO: 296 - protein, 10 aa, synthetic construct
VLPELRSVFS

SEQ ID NO: 297 - protein, 8 aa, synthetic construct
VLSKQMSF

SEQ ID NO: 298 - protein, 7 aa, synthetic construct
VPAGRRS

SEQ ID NO: 299 - protein, 8 aa, synthetic construct
VPAGRRSL

SEQ ID NO: 300 - protein, 4 aa, synthetic construct
VPRQ

SEQ ID NO: 301 - protein, 5 aa, synthetic construct
VPRQV

SEQ ID NO: 302 - protein, 5 aa, synthetic construct
VSRSA

SEQ ID NO: 303 - protein, 8 aa, synthetic construct
VVPEGRRS

SEQ ID NO: 304 - protein, 8 aa, synthetic construct
WATPRPMR

SEQ ID NO: 305 - protein, 8 aa, synthetic construct
WDHPISLL

SEQ ID NO: 306 - skipped

SEQ ID NO: 307 - protein, 10 aa, synthetic construct
YDPZVKVVLA

SEQ ID NO: 308 - protein, 8 aa, synthetic construct
YGAGLGVV

SEQ ID NO: 309 - protein, 7 aa, synthetic construct
YIVSRSA

SEQ ID NO: 310 - protein, 8 aa, synthetic construct
YKKFVGSL

SEQ ID NO: 311 - protein, 10 aa, synthetic construct
YVPRVKALEM

SEQ ID NO: 312 - protein, 8 aa, synthetic construct
AQNLLGMY

SEQ ID NO: 313 - protein, 7 aa, synthetic construct
VAQFVLT

SEQ ID NO: 314 - protein, 8 aa, synthetic construct
HMMQYARH

SEQ ID NO: 315 - protein, 221 aa, Homo sapiens
CPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 316 - protein, 221 aa, Homo sapiens
CPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLG

SEQ ID NO: 317 - skipped

SEQ ID NO: 318 - protein, 6 aa, synthetic construct
GSSGGS

SEQ ID NO: 319 - protein, 4 aa, synthetic construct
ESKY

SEQ ID NO: 320 - skipped

SEQ ID NO: 321 - protein, 4 aa, synthetic construct
SGGG

SEQ ID NO: 322 - protein, 5 aa, synthetic construct
PGGGS

SEQ ID NO: 323 - protein, 5 aa, synthetic construct
PGPPS

SEQ ID NO: 324 - protein, 5 aa, synthetic construct
GGPPS

SEQ ID NO: 325 - protein, 5 aa, synthetic construct
GCPPC

SEQ ID NO: 326 - protein, 5 aa, synthetic construct
PCPPC

SEQ ID NO: 327 - protein, 5 aa, synthetic construct
GPGGS

SEQ ID NO: 328-334 - skipped (7 entries)

SEQ ID NO: 335 - protein, 5 aa, synthetic construct
SGGGG

SEQ ID NO: 336-342 - skipped (7 entries)

SEQ ID NO: 343 - protein, 17 aa, synthetic construct
MRAWIFFLLCLAGRALA

SEQ ID NO: 344 - protein, 23 aa, synthetic construct
MALTFALLVALLVLSCKSSCSVG

SEQ ID NO: 345 - protein, 20 aa, synthetic construct
METDTLLLWVLLLWVPGSTG

SEQ ID NO: 346 - skipped

SEQ ID NO: 347 - protein, 113 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINT

SEQ ID NO: 348 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 349-355 - skipped (7 entries)

SEQ ID NO: 356 - protein, 342 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 357 - skipped

SEQ ID NO: 358 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 359 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 360 - protein, 67 aa, Homo sapiens
ITCPPPMSVEHADIWVKSYSLYSRERYICNSGFKRKAGTSSLTECVLNKATNVAHWTTPSLKCIRDP

SEQ ID NO: 361 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 362 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 363 - protein, 102 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAAS

SEQ ID NO: 364 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 365 - protein, 214 aa, Homo sapiens
AVNGTSQFTCFYNSRANISCVWSQDGALQDTSCQVHAWPDRRRWNQTCELLPVSQASWACNLILGAPESQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 366 - protein, 214 aa, Homo sapiens
AVNGTSQFTCFYNSRANISCVWSQDGALQDTSCQVHAWPDRRRWNQTCELLPVSQASWACNLILGAPDHQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 367 - protein, 214 aa, Homo sapiens
AVNGTSQFTCFYNSRANISCVWSQDGALQDTSCQVHAWPDRRRWNQTCELLPVSQASWACNLILGAPDSQKLTTQDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 368 - protein, 214 aa, Homo sapiens
AVNGTSQFTCFYNSRANISCVWSQDGALQDTSCQVHAWPDRRRWNQTCELLPVSQASWACNLILGAPDSQKLTTFDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 369 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFQRHLEFEARTLSPGHTWEEAPLLTLKQKQEWICLETLTPDTQYEFQVRVKPLQGEFTTWSPWSQPLAFRTKPAALGKDT

SEQ ID NO: 370 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFQRRLEFEARTLSPGHTWEEAPLLTLKQKQEWICLETLTPDTQYEFQVRVKPLQGEFTTWSPWSQPLAFRTKPAALGKDT

SEQ ID NO: 371 - protein, 214 aa, Homo sapiens
AVNGTSQFTCFYNSYANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 372 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 373 - protein, 213 aa, Homo sapiens
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 374 - protein, 78 aa, Homo sapiens
ITCPPPMSVEHADIWVKSYSLYSRERYICNSGFKRKAGTSSLTECVLNKATNVAHWTTPSLKCIRDPALVHQRPAPPS

SEQ ID NO: 375 - protein, 6 aa, synthetic construct
QGQSGS

SEQ ID NO: 376 - protein, 5 aa, synthetic construct
GQSGS

SEQ ID NO: 377 - protein, 4 aa, synthetic construct
QSGS

SEQ ID NO: 378 - protein, 7 aa, synthetic construct
QGQSGQG

SEQ ID NO: 379 - protein, 6 aa, synthetic construct
GQSGQG

SEQ ID NO: 380 - protein, 5 aa, synthetic construct
QSGQG

SEQ ID NO: 381 - protein, 4 aa, synthetic construct
SGQG

SEQ ID NO: 382 - protein, 6 aa, synthetic construct
QGQSGQ

SEQ ID NO: 383 - protein, 5 aa, synthetic construct
GQSGQ

SEQ ID NO: 384 - protein, 4 aa, synthetic construct
QSGQ

SEQ ID NO: 385 - protein, 5 aa, synthetic construct
QGQSG

SEQ ID NO: 386 - protein, 4 aa, synthetic construct
QGQS

SEQ ID NO: 387 - protein, 10 aa, synthetic construct
EPKSCDKTHT

SEQ ID NO: 388 - protein, 12 aa, synthetic construct
ELKTPLGDTTHT

SEQ ID NO: 389 - protein, 7 aa, synthetic construct
ESKYGPP

SEQ ID NO: 390 - skipped

SEQ ID NO: 391 - protein, 426 aa, synthetic construct
TNTRFSVDEVTGGGGSSVASRSNKGGGISSGLLSGRSDNPGGSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGISSGLLSGRSDNPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 392 - protein, 447 aa, synthetic construct
VDEVYDYHQGGGGSLLLGSTGGGGSISSGLLSGRSDNPGGGGSYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDGGISSGLLSGRSDNPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 393 - protein, 419 aa, synthetic construct
EEFAVLRDGKGGGGSGVLNVWGVGGISSGLLSGRSDNPGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQGGISSGLLSGRSDNPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 394 - protein, 371 aa, synthetic construct
KLTTVRGGGGSASHYFERGGLSGRSNIGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 395 - protein, 376 aa, synthetic construct
SQKLTTVDGGGGSASHYFERHLEGGLSGRSNIGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 396 - protein, 376 aa, synthetic construct
SQKLTTVDGGGGSASHYFERHLEGGLSGRSNIGGNWVNVISDLKKIEDLIQSMHIDATLYTESDVHPSCKVTAMKCFLDELQVISDESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 397 - protein, 376 aa, synthetic construct
SQKLTTVRGGGGSASHYFERHLEGGLSGRSNIGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 398 - protein, 376 aa, synthetic construct
SQKLTTVRGGGGSASHYFERHLEGGLSGRSNIGGNWVNVISDLKKIEDLIQSMHIDATLYTESDVHPSCKVTAMKCFLDELQVISDESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 399 - protein, 371 aa, synthetic construct
KLTTVRGGGGSASHYFERGGLSGRSNIGGNWVNVISDLKKIEDLIQSMHIDATLYTESDVHPSCKVTAMKCFLDELQVISDESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 400 - protein, 371 aa, synthetic construct
ALTTVDGGGGSASHYFERGGLSGRSNIGGNWVNVISDLKKIEDLIQSMHIDATLYTESDVHPSCKVTAMKCFLNELQVISNESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 401 - skipped

SEQ ID NO: 402 - protein, 114 aa, Homo sapiens
  VARIANT 45: note = X is any amino acid that is not L
  VARIANT 52: note = X is any amino acid
NWVNVISDLKKIEDLIQSMHIDATLYTESDVHPSCKVTAMKCFLXELQVISXESGDASIHDTVENLIILANNSLSSNGNVTESGCKECEELEEKNIKEFLQSFVHIVQMFINTS

SEQ ID NO: 403 - protein, 114 aa, Homo sapiens; identical to SEQ ID NO: 402
  VARIANT 45: note = X is D, N, or T
  VARIANT 52: note = X is any amino acid

SEQ ID NO: 404 - protein, 114 aa, Homo sapiens
  VARIANT 45: note = X is any amino acid that is not L
NWVNVISDLKKIEDLIQSMHIDATLYTESDVHPSCKVTAMKCFLXELQVISLESGDASIHDTVENLIILANNSLSSNGNVTESGCKECEELEEKNIKEFLQSFVHIVQMFINTS

SEQ ID NO: 405 - protein, 114 aa, Homo sapiens
NWVNVISDLKKIEDLIQSMHIDATLYTESDVHPSCKVTAMKCFLDELQVISLESGDASIHDTVENLIILANNSLSSNGNVTESGCKECEELEEKNIKEFLQSFVHIVQMFINTS

SEQ ID NO: 406 - protein, 114 aa, Homo sapiens
NWVNVISDLKKIEDLIQSMHIDATLYTESDVHPSCKVTAMKCFLNELQVISLESGDASIHDTVENLIILANNSLSSNGNVTESGCKECEELEEKNIKEFLQSFVHIVQMFINTS

SEQ ID NO: 407 - protein, 114 aa, Homo sapiens
NWVNVISDLKKIEDLIQSMHIDATLYTESDVHPSCKVTAMKCFLTELQVISLESGDASIHDTVENLIILANNSLSSNGNVTESGCKECEELEEKNIKEFLQSFVHIVQMFINTS

SEQ ID NO: 408 - protein, 114 aa, Homo sapiens; identical to SEQ ID NO: 402
  VARIANT 45: note = X is any amino acid that is not L
  VARIANT 52: note = X is any amino acid that is not L

SEQ ID NO: 409 - protein, 114 aa, Homo sapiens; identical to SEQ ID NO: 402
  VARIANT 45: note = X is D, N, or T
  VARIANT 52: note = X is any amino acid that is not L

SEQ ID NO: 410 - protein, 114 aa, Homo sapiens; identical to SEQ ID NO: 402
  VARIANT 45: note = X is D, N, or T
  VARIANT 52: note = X is D, N, or T

SEQ ID NO: 411 - protein, 114 aa, Homo sapiens
NWVNVISDLKKIEDLIQSMHIDATLYTESDVHPSCKVTAMKCFLDELQVISDESGDASIHDTVENLIILANNSLSSNGNVTESGCKECEELEEKNIKEFLQSFVHIVQMFINTS

SEQ ID NO: 412 - protein, 114 aa, Homo sapiens
NWVNVISDLKKIEDLIQSMHIDATLYTESDVHPSCKVTAMKCFLDELQVISNESGDASIHDTVENLIILANNSLSSNGNVTESGCKECEELEEKNIKEFLQSFVHIVQMFINTS

SEQ ID NO: 413 - protein, 114 aa, Homo sapiens
NWVNVISDLKKIEDLIQSMHIDATLYTESDVHPSCKVTAMKCFLDELQVISTESGDASIHDTVENLIILANNSLSSNGNVTESGCKECEELEEKNIKEFLQSFVHIVQMFINTS

SEQ ID NO: 414 - protein, 114 aa, Homo sapiens
NWVNVISDLKKIEDLIQSMHIDATLYTESDVHPSCKVTAMKCFLNELQVISDESGDASIHDTVENLIILANNSLSSNGNVTESGCKECEELEEKNIKEFLQSFVHIVQMFINTS

SEQ ID NO: 415 - protein, 114 aa, Homo sapiens
NWVNVISDLKKIEDLIQSMHIDATLYTESDVHPSCKVTAMKCFLNELQVISNESGDASIHDTVENLIILANNSLSSNGNVTESGCKECEELEEKNIKEFLQSFVHIVQMFINTS

SEQ ID NO: 416 - protein, 114 aa, Homo sapiens
NWVNVISDLKKIEDLIQSMHIDATLYTESDVHPSCKVTAMKCFLNELQVISTESGDASIHDTVENLIILANNSLSSNGNVTESGCKECEELEEKNIKEFLQSFVHIVQMFINTS

SEQ ID NO: 417 - protein, 114 aa, Homo sapiens
NWVNVISDLKKIEDLIQSMHIDATLYTESDVHPSCKVTAMKCFLTELQVISDESGDASIHDTVENLIILANNSLSSNGNVTESGCKECEELEEKNIKEFLQSFVHIVQMFINTS

SEQ ID NO: 418 - protein, 114 aa, Homo sapiens
NWVNVISDLKKIEDLIQSMHIDATLYTESDVHPSCKVTAMKCFLTELQVISNESGDASIHDTVENLIILANNSLSSNGNVTESGCKECEELEEKNIKEFLQSFVHIVQMFINTS

SEQ ID NO: 419 - protein, 114 aa, Homo sapiens
NWVNVISDLKKIEDLIQSMHIDATLYTESDVHPSCKVTAMKCFLTELQVISTESGDASIHDTVENLIILANNSLSSNGNVTESGCKECEELEEKNIKEFLQSFVHIVQMFINTS

SEQ ID NO: 420 - protein, 114 aa, Homo sapiens
NWVNVISDLKKIEDLIQSMHIDATLYTESDVHPSCKVTAMKCFLLELQVISDESGDASIHDTVENLIILANNSLSSNGNVTESGCKECEELEEKNIKEFLQSFVHIVQMFINTS

SEQ ID NO: 421 - protein, 114 aa, Homo sapiens
NWVNVISDLKKIEDLIQSMHIDATLYTESDVHPSCKVTAMKCFLLELQVISNESGDASIHDTVENLIILANNSLSSNGNVTESGCKECEELEEKNIKEFLQSFVHIVQMFINTS

SEQ ID NO: 422 - protein, 114 aa, Homo sapiens
NWVNVISDLKKIEDLIQSMHIDATLYTESDVHPSCKVTAMKCFLLELQVISTESGDASIHDTVENLIILANNSLSSNGNVTESGCKECEELEEKNIKEFLQSFVHIVQMFINTS

SEQ ID NO: 423 - protein, 342 aa, synthetic construct
NWVNVISDLKKIEDLIQSMHIDATLYTESDVHPSCKVTAMKCFLDELQVISLESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 424 - protein, 342 aa, synthetic construct
NWVNVISDLKKIEDLIQSMHIDATLYTESDVHPSCKVTAMKCFLNELQVISLESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 425 - protein, 342 aa, synthetic construct
NWVNVISDLKKIEDLIQSMHIDATLYTESDVHPSCKVTAMKCFLTELQVISLESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 426 - protein, 342 aa, synthetic construct
NWVNVISDLKKIEDLIQSMHIDATLYTESDVHPSCKVTAMKCFLLELQVISDESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 427 - protein, 342 aa, synthetic construct
NWVNVISDLKKIEDLIQSMHIDATLYTESDVHPSCKVTAMKCFLLELQVISNESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 428 - protein, 342 aa, synthetic construct
NWVNVISDLKKIEDLIQSMHIDATLYTESDVHPSCKVTAMKCFLLELQVISTESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 429 - protein, 342 aa, synthetic construct
NWVNVISDLKKIEDLIQSMHIDATLYTESDVHPSCKVTAMKCFLDELQVISDESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 430 - protein, 342 aa, synthetic construct
NWVNVISDLKKIEDLIQSMHIDATLYTESDVHPSCKVTAMKCFLNELQVISNESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 431 - protein, 342 aa, synthetic construct
NWVNVISDLKKIEDLIQSMHIDATLYTESDVHPSCKVTAMKCFLTELQVISTESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 432 - protein, 8 aa, synthetic construct
HTGRSGAL

SEQ ID NO: 433 - protein, 5 aa, synthetic construct
HVPRQ

SEQ ID NO: 434 - protein, 6 aa, synthetic construct
HVPRQV

SEQ ID NO: 435 - protein, 11 aa, synthetic construct
HVPRQVAPRSF

SEQ ID NO: 436 - protein, 11 aa, synthetic construct
HVPRQVLSGRS

SEQ ID NO: 437 - protein, 13 aa, synthetic construct
HVPRQVLSGRSAN

SEQ ID NO: 438 - protein, 8 aa, synthetic construct
HWHLGPPT

SEQ ID NO: 439 - protein, 8 aa, synthetic construct
IANLLSMV

SEQ ID NO: 440 - protein, 4 aa, synthetic construct
IDGR

SEQ ID NO: 441 - protein, 4 aa, synthetic construct
IEGR

SEQ ID NO: 442 - protein, 8 aa, synthetic construct
ILNLLSMV

SEQ ID NO: 443 - protein, 8 aa, synthetic construct
ILPRSPAF

SEQ ID NO: 444 - protein, 8 aa, synthetic construct
IPFSWSRF

SEQ ID NO: 445 - protein, 8 aa, synthetic construct
IQNLLSMV

SEQ ID NO: 446 - protein, 5 aa, synthetic construct
ISSGL

SEQ ID NO: 447 - protein, 6 aa, synthetic construct
ISSGLL

SEQ ID NO: 448 - protein, 7 aa, synthetic construct
ISSGLLS

SEQ ID NO: 449 - protein, 13 aa, synthetic construct
ISSGLLSGRSANI

SEQ ID NO: 450 - protein, 13 aa, synthetic construct
ISSGLLSGRSANP

SEQ ID NO: 451 - protein, 15 aa, synthetic construct
ISSGLLSGRSANPRG

SEQ ID NO: 452 - protein, 13 aa, synthetic construct
ISSGLLSGRSDDH

SEQ ID NO: 453 - protein, 13 aa, synthetic construct
ISSGLLSGRSDIH

SEQ ID NO: 454 - protein, 13 aa, synthetic construct
ISSGLLSGRSDNH

SEQ ID NO: 455 - protein, 13 aa, synthetic construct
ISSGLLSGRSDNI

SEQ ID NO: 456 - protein, 13 aa, synthetic construct
ISSGLLSGRSDNP

SEQ ID NO: 457 - protein, 13 aa, synthetic construct
ISSGLLSGRSDQH

SEQ ID NO: 458 - protein, 13 aa, synthetic construct
ISSGLLSGRSDTH

SEQ ID NO: 459 - protein, 13 aa, synthetic construct
ISSGLLSGRSDYH

SEQ ID NO: 460 - protein, 13 aa, synthetic construct
ISSGLLSGRSGNH

SEQ ID NO: 461 - protein, 12 aa, synthetic construct
ISSGLLSGRSNI

SEQ ID NO: 462 - protein, 13 aa, synthetic construct
ISSGLLSGRSNIG

SEQ ID NO: 463 - protein, 14 aa, synthetic construct
ISSGLLSGRSNIGS

SEQ ID NO: 464 - protein, 8 aa, synthetic construct
ISSGLLSS

SEQ ID NO: 465 - protein, 22 aa, synthetic construct
ISSGLLSSGGSGGSLSGRSDNH

SEQ ID NO: 466 - protein, 22 aa, synthetic construct
ISSGLLSSGGSGGSLSGRSGNH

SEQ ID NO: 467 - protein, 7 aa, synthetic construct
ISSGLSS

SEQ ID NO: 468 - protein, 6 aa, synthetic construct
IVSRSA

SEQ ID NO: 469 - protein, 12 aa, synthetic construct
KGLTGRSDRHQA

SEQ ID NO: 470 - protein, 10 aa, synthetic construct
KGPKVKVVTL

SEQ ID NO: 471 - protein, 12 aa, synthetic construct
KNLYGRSENNGN

SEQ ID NO: 472 - protein, 8 aa, synthetic construct
KRMPVQFL

SEQ ID NO: 473 - protein, 8 aa, synthetic construct
LAAPLGLL

SEQ ID NO: 474 - protein, 4 aa, synthetic construct
LAHG

SEQ ID NO: 475 - protein, 5 aa, synthetic construct
LAHGL

SEQ ID NO: 476 - protein, 6 aa, synthetic construct
LAHGLF

SEQ ID NO: 477 - protein, 9 aa, synthetic construct
LAPLGLQRR

SEQ ID NO: 478 - protein, 5 aa, synthetic construct
LARAG

SEQ ID NO: 479 - protein, 6 aa, synthetic construct
LARAGI

SEQ ID NO: 480 - protein, 6 aa, synthetic construct
LARAGL

SEQ ID NO: 481 - protein, 8 aa, synthetic construct
LKAAPRWA

SEQ ID NO: 482 - protein, 8 aa, synthetic construct
LKAAPRWF

SEQ ID NO: 483 - protein, 8 aa, synthetic construct
LKAAPVWA

SEQ ID NO: 484 - protein, 8 aa, synthetic construct
LKAAPVWF

SEQ ID NO: 485 - protein, 8 aa, synthetic construct
LKGRSYYY

SEQ ID NO: 486 - protein, 8 aa, synthetic construct
LLAPSHRA

SEQ ID NO: 487 - protein, 8 aa, synthetic construct
LLEALRAL

SEQ ID NO: 488 - protein, 8 aa, synthetic construct
LLESLRAL

SEQ ID NO: 489 - protein, 8 aa, synthetic construct
LLLPAHGG

SEQ ID NO: 490 - protein, 8 aa, synthetic construct
LLLPLLGS

SEQ ID NO: 491 - protein, 8 aa, synthetic construct
LLNALRAL

SEQ ID NO: 492 - protein, 8 aa, synthetic construct
LLNSLRAL

SEQ ID NO: 493 - protein, 8 aa, synthetic construct
LLQALRAL

SEQ ID NO: 494 - protein, 8 aa, synthetic construct
LLQSLRAL

SEQ ID NO: 495 - protein, 8 aa, synthetic construct
LLSALRAL

SEQ ID NO: 496 - protein, 8 aa, synthetic construct
LLSSLRAL

SEQ ID NO: 497 - protein, 8 aa, synthetic construct
LNGRSDNH

SEQ ID NO: 498 - protein, 7 aa, synthetic construct
LPAGLLL

SEQ ID NO: 499 - protein, 8 aa, synthetic construct
LPAGLLLR

SEQ ID NO: 500 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 501 - protein, 33 aa, synthetic construct
  VARIANT 6: note = X is D, K, or R
  VARIANT 8..26: note = X is any amino acid or absent
  VARIANT 33: note = X is R or K
ALTTVXXXXXXXXXXXXXXXXXXXXXASHYFEX

SEQ ID NO: 502 - protein, 33 aa, synthetic construct
  VARIANT 8..26: note = X is any amino acid or absent
  VARIANT 33: note = X is R or K
ALTTVDXXXXXXXXXXXXXXXXXXXXASHYFEX

SEQ ID NO: 503 - protein, 33 aa, synthetic construct
  VARIANT 8..26: note = X is any amino acid or absent
  VARIANT 33: note = X is R or K
ALTTVKXXXXXXXXXXXXXXXXXXXXASHYFEX

SEQ ID NO: 504 - protein, 33 aa, synthetic construct
  VARIANT 8..26: note = X is any amino acid or absent
  VARIANT 33: note = X is R or K
ALTTVRXXXXXXXXXXXXXXXXXXXXASHYFEX

SEQ ID NO: 505 - protein, 18 aa, synthetic construct
  VARIANT 18: note = X is R of K
ALTTVDGGGGSASHYFEX

SEQ ID NO: 506 - protein, 18 aa, synthetic construct
  VARIANT 18: note = X is R or K
ALTTVKGGGGSASHYFEX

SEQ ID NO: 507 - protein, 18 aa, synthetic construct
  VARIANT 18: note = X is R or K
ALTTVRGGGGSASHYFEX

SEQ ID NO: 508 - protein, 32 aa, synthetic construct
  VARIANT 6: note = X is D, K, or R
  VARIANT 8..26: note = X is any amino acid or absent
ALTTVXXXXXXXXXXXXXXXXXXXXXASHYFE

SEQ ID NO: 509 - protein, 32 aa, synthetic construct
  VARIANT 8..26: note = X is any amino acid or absent
ALTTVDXXXXXXXXXXXXXXXXXXXXASHYFE

SEQ ID NO: 510 - protein, 32 aa, synthetic construct
  VARIANT 8..26: note = X is any amino acid or absent
ALTTVKXXXXXXXXXXXXXXXXXXXXASHYFE

SEQ ID NO: 511 - protein, 32 aa, synthetic construct
  VARIANT 8..26: note = X is any amino acid or absent
ALTTVRXXXXXXXXXXXXXXXXXXXXASHYFE

SEQ ID NO: 512 - protein, 17 aa, synthetic construct
ALTTVDGGGGSASHYFE

SEQ ID NO: 513 - protein, 17 aa, synthetic construct
ALTTVKGGGGSASHYFE

SEQ ID NO: 514 - protein, 17 aa, synthetic construct
ALTTVRGGGGSASHYFE

SEQ ID NO: 515 - protein, 32 aa, synthetic construct
  VARIANT 1: note = X is any amino acid or absent
  VARIANT 6: note = X is D, K, or R
  VARIANT 8..26: note = X is any amino acid or absent
XLTTVXXXXXXXXXXXXXXXXXXXXXASHYFE

SEQ ID NO: 516 - protein, 32 aa, synthetic construct
  VARIANT 1: note = X is any amino acid or absent
  VARIANT 8..26: note = X is any amino acid or absent
XLTTVDXXXXXXXXXXXXXXXXXXXXASHYFE

SEQ ID NO: 517 - protein, 32 aa, synthetic construct
  VARIANT 1: note = X is any amino acid or absent
  VARIANT 8..26: note = X is any amino acid or absent
XLTTVKXXXXXXXXXXXXXXXXXXXXASHYFE

SEQ ID NO: 518 - protein, 32 aa, synthetic construct
  VARIANT 1: note = X is any amino acid or absent
  VARIANT 8..26: note = X is any amino acid or absent
XLTTVRXXXXXXXXXXXXXXXXXXXXASHYFE

SEQ ID NO: 519 - skipped

SEQ ID NO: 520 - protein, 65 aa, Homo sapiens; identical to SEQ ID NO: 361

SEQ ID NO: 521 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 522 - protein, 146 aa, Mus musculus [sequence omitted]

SEQ ID NO: 523 - protein, 371 aa, synthetic construct
ALTTVDGGGGSASHYFERGGLSGRSNIGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 524 - protein, 391 aa, synthetic construct
METDTLLLWVLLLWVPGSTGALTTVDGGGGSASHYFERGGLSGRSNIGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGLSGRSNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 525 - protein, 371 aa, synthetic construct; identical to SEQ ID NO: 523

SEQ ID NO: 526 - protein, 97 aa, synthetic construct
ITCPPPMSVEHADIWVKSYSLYSRERYICNSGFKRKAGTSSLTECVLNKATNVAHWTTPSLKCIRDPALVHQRPAPPGGGGSGGGGSHHHHHHHHHH

SEQ ID NO: 527 - protein, 374 aa, synthetic construct
ALTTVDGGGGSASHYFERGGLSGRSNIGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGLSGRSNICPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNRFTQKSLSLSLGK

SEQ ID NO: 528 - protein, 303 aa, synthetic construct
ITCPPPMSVEHADIWVKSYSLYSRERYICNSGFKRKAGTSSLTECVLNKATNVAHWTTPSLKCIRGGLSGRSNIESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 529 - DNA, 1176 bp, synthetic construct
  source 1..1176: mol_type = other DNA
atggaaaccgacacactgctgctgtgggtgctgcttttgtgggtgccaggatccacaggcgccctgacaacagttgatggtggcggaggaagcgccagccactactttgaaagaggcggcctgagcggcagaagcaacatcggcggaaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcggcggactgtccggccggtccaatatttgtcctccttgtcctgctcctgagttcctcggcggaccttccgtgttcctgtttcctccaaagcctaaggacaccctgatgatcagcagaacccctgaagtgacctgcgtggtggtcgacgtttcacaagaggaccccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagggaaccccaggtttacacactgccacctagccaagaggaaatgaccaagaaccaggtgtccctgacctgcctggtcaagggcttttacccctccgatatcgccgtggaatgggagagcaatggccagcctgagaacaactacaagaccacacctcctgtgctggacagcgacggctcattcttcctgtacagcagactgaccgtggacaagagcagatggcagcagggcaacgtgttcagctgcagcgtgatgcacgaggccctgcacaaccactacacccagaagtctctgagcctgagctga

SEQ ID NO: 530 - protein, 8 aa, synthetic construct
LPAHLVLL

SEQ ID NO: 531 - protein, 8 aa, synthetic construct
LPAHLVLV

SEQ ID NO: 532 - protein, 8 aa, synthetic construct
LPGGLSPW

SEQ ID NO: 533 - protein, 8 aa, synthetic construct
LPSHLVLL

SEQ ID NO: 534 - protein, 8 aa, synthetic construct
LPSHLVLV

SEQ ID NO: 535 - protein, 5 aa, synthetic construct
LPTFV

SEQ ID NO: 536 - protein, 6 aa, synthetic construct
LPTFVH

SEQ ID NO: 537 - protein, 7 aa, synthetic construct
LPTFVHL

SEQ ID NO: 538 - protein, 5 aa, synthetic construct
LRSGW

SEQ ID NO: 539 - protein, 4 aa, synthetic construct
LSGR

SEQ ID NO: 540 - protein, 5 aa, synthetic construct
LSGRS

SEQ ID NO: 541 - protein, 6 aa, synthetic construct
LSGRSA

SEQ ID NO: 542 - protein, 12 aa, synthetic construct
LSGRSALAHGLF

SEQ ID NO: 543 - protein, 7 aa, synthetic construct
LSGRSAN

SEQ ID NO: 544 - protein, 8 aa, synthetic construct
LSGRSANI

SEQ ID NO: 545 - protein, 8 aa, synthetic construct
LSGRSANP

SEQ ID NO: 546 - protein, 6 aa, synthetic construct
LSGRSD

SEQ ID NO: 547 - protein, 7 aa, synthetic construct
LSGRSDD

SEQ ID NO: 548 - protein, 8 aa, synthetic construct
LSGRSDDH

SEQ ID NO: 549 - protein, 7 aa, synthetic construct
LSGRSDI

SEQ ID NO: 550 - protein, 8 aa, synthetic construct
LSGRSDIH

SEQ ID NO: 551 - protein, 7 aa, synthetic construct
LSGRSDN

SEQ ID NO: 552 - protein, 8 aa, synthetic construct
LSGRSDNH

SEQ ID NO: 553 - protein, 7 aa, synthetic construct
LSGRSNI

SEQ ID NO: 554 - protein, 18 aa, synthetic construct
LSGRSDNHGGAVGLLAPP

SEQ ID NO: 555 - protein, 22 aa, synthetic construct
LSGRSDNHGGSGGSISSGLLSS

SEQ ID NO: 556 - protein, 22 aa, synthetic construct
LSGRSDNHGGSGGSQNQALRMA

SEQ ID NO: 557 - protein, 20 aa, synthetic construct
LSGRSDNHGGVHMPLGFLGP

SEQ ID NO: 558 - protein, 8 aa, synthetic construct
LSGRSDNI

SEQ ID NO: 559 - protein, 8 aa, synthetic construct
LSGRSDNP

SEQ ID NO: 560 - protein, 7 aa, synthetic construct
LSGRSDQ

SEQ ID NO: 561 - protein, 8 aa, synthetic construct
LSGRSDQH

SEQ ID NO: 562 - protein, 7 aa, synthetic construct
LSGRSDT

SEQ ID NO: 563 - protein, 8 aa, synthetic construct
LSGRSDTH

SEQ ID NO: 564 - protein, 7 aa, synthetic construct
LSGRSDY

SEQ ID NO: 565 - protein, 8 aa, synthetic construct
LSGRSDYH

SEQ ID NO: 566 - protein, 8 aa, synthetic construct
LSGRSENH

SEQ ID NO: 567 - protein, 6 aa, synthetic construct
LSGRSG

SEQ ID NO: 568 - protein, 7 aa, synthetic construct
LSGRSGN

SEQ ID NO: 569 - protein, 8 aa, synthetic construct
LSGRSGNH

SEQ ID NO: 570 - protein, 22 aa, synthetic construct
LSGRSGNHGGSGGSISSGLLSS

SEQ ID NO: 571 - protein, 22 aa, synthetic construct
LSGRSGNHGGSGGSQNQALRMA

SEQ ID NO: 572 - protein, 8 aa, synthetic construct
LSGRSGNP

SEQ ID NO: 573 - protein, 8 aa, synthetic construct
LSGRSVTQ

SEQ ID NO: 574 - protein, 8 aa, synthetic construct
LSQARWRK

SEQ ID NO: 575 - protein, 8 aa, synthetic construct
LTFPTYIF

SEQ ID NO: 576 - protein, 8 aa, synthetic construct
LTFPTYWF

SEQ ID NO: 577 - protein, 8 aa, synthetic construct
LTGRSDRH

SEQ ID NO: 578 - protein, 7 aa, synthetic construct
LTGRSGA

SEQ ID NO: 579 - protein, 7 aa, synthetic construct
LTGRSGA

SEQ ID NO: 580 - protein, 8 aa, synthetic construct
LYAAPRWA

SEQ ID NO: 581 - protein, 8 aa, synthetic construct
LYAAPRWF

SEQ ID NO: 582 - protein, 8 aa, synthetic construct
LYAAPVWA

SEQ ID NO: 583 - protein, 8 aa, synthetic construct
LYAAPVWF

SEQ ID NO: 584 - protein, 8 aa, synthetic construct
LYGRSENN

SEQ ID NO: 585 - protein, 8 aa, synthetic construct
MDAFLESS

SEQ ID NO: 586 - protein, 8 aa, synthetic construct
MGLFSEAG

SEQ ID NO: 587 - protein, 6 aa, synthetic construct
MGPWFM

SEQ ID NO: 588 - protein, 8 aa, synthetic construct
MIAPVAYR

SEQ ID NO: 589 - protein, 6 aa, synthetic construct
MLRSGW

SEQ ID NO: 590 - protein, 7 aa, synthetic construct
MLRSGWR

SEQ ID NO: 591 - protein, 8 aa, synthetic construct
MLRSGWRG

SEQ ID NO: 592 - protein, 8 aa, synthetic construct
MLRSGWRL

SEQ ID NO: 593 - protein, 8 aa, synthetic construct
MLRSGWRS

SEQ ID NO: 594 - protein, 8 aa, synthetic construct
MTFPTYIF

SEQ ID NO: 595 - protein, 8 aa, synthetic construct
MTFPTYWF

SEQ ID NO: 596 - protein, 12 aa, synthetic construct
NHRIGRSDNHRR

SEQ ID NO: 597 - protein, 10 aa, synthetic construct
NMPSFKLVTG

SEQ ID NO: 598 - protein, 12 aa, synthetic construct
NTLSGRSENHSG

SEQ ID NO: 599 - protein, 12 aa, synthetic construct
NTLSGRSGNHGS

SEQ ID NO: 600 - protein, 17 aa, synthetic construct
  VARIANT 1: note = X is any amino acid or absent
XLTTVDGGGGSASHYFE

SEQ ID NO: 601 - protein, 17 aa, synthetic construct
  VARIANT 1: note = X is any amino acid or absent
XLTTVKGGGGSASHYFE

SEQ ID NO: 602 - protein, 17 aa, synthetic construct
  VARIANT 1: note = X is any amino acid or absent
XLTTVRGGGGSASHYFE

SEQ ID NO: 603 - protein, 10 aa, synthetic construct
NZPRVRLVLP

SEQ ID NO: 604 - protein, 8 aa, synthetic construct
PAGLWLDP

SEQ ID NO: 605 - protein, 5 aa, synthetic construct
PAGRR

SEQ ID NO: 606 - protein, 6 aa, synthetic construct
PAGRRS

SEQ ID NO: 607 - protein, 7 aa, synthetic construct
PAGRRSL

SEQ ID NO: 608 - protein, 8 aa, synthetic construct
PASLWYTQ

SEQ ID NO: 609 - protein, 8 aa, synthetic construct
PESRRWMP

SEQ ID NO: 610 - protein, 6 aa, synthetic construct
PFHLSR

SEQ ID NO: 611 - protein, 6 aa, synthetic construct
PHGFFQ

SEQ ID NO: 612 - protein, 7 aa, synthetic construct
PLARAGI

SEQ ID NO: 613 - protein, 7 aa, synthetic construct
PLARAGL

SEQ ID NO: 614 - protein, 4 aa, synthetic construct
PLGL

SEQ ID NO: 615 - protein, 6 aa, synthetic construct
PLGLAG

SEQ ID NO: 616 - protein, 6 aa, synthetic construct
PLGLWA

SEQ ID NO: 617 - protein, 7 aa, synthetic construct
PLGVRGK

SEQ ID NO: 618 - protein, 8 aa, synthetic construct
PLTGRSGG

SEQ ID NO: 619 - protein, 12 aa, synthetic construct
PLTGRSGGGGSS

SEQ ID NO: 620 - protein, 5 aa, synthetic construct
PPLGR

SEQ ID NO: 621 - protein, 10 aa, synthetic construct
PPPDMKLFPG

SEQ ID NO: 622 - protein, 10 aa, synthetic construct
PPPEVRSFSV

SEQ ID NO: 623 - protein, 10 aa, synthetic construct
PPPVLKLLEW

SEQ ID NO: 624 - protein, 12 aa, synthetic construct
PPSIARSDNLAN

SEQ ID NO: 625 - protein, 8 aa, synthetic construct
PQHRIVSF

SEQ ID NO: 626 - protein, 8 aa, synthetic construct
PRFKIIGG

SEQ ID NO: 627 - protein, 8 aa, synthetic construct
PRFRIIGG

SEQ ID NO: 628 - protein, 10 aa, synthetic construct
PRPFVKSVDQ

SEQ ID NO: 629 - protein, 4 aa, synthetic construct
PRQV

SEQ ID NO: 630 - protein, 4 aa, synthetic construct
PRSF

SEQ ID NO: 631 - protein, 10 aa, synthetic construct
PSPPVKMMPE

SEQ ID NO: 632 - protein, 10 aa, synthetic construct
PTNGGSGGSS

SEQ ID NO: 633 - protein, 10 aa, synthetic construct
RVPKVKVMLD

SEQ ID NO: 634 - protein, 8 aa, synthetic construct
SAGFSLPA

SEQ ID NO: 635 - protein, 8 aa, synthetic construct
SAPAVESE

SEQ ID NO: 636 - protein, 10 aa, synthetic construct
SAPYFRMMDM

SEQ ID NO: 637 - protein, 8 aa, synthetic construct
SARGPSRW

SEQ ID NO: 638 - protein, 8 aa, synthetic construct
SCGPPLGR

SEQ ID NO: 639 - protein, 8 aa, synthetic construct
SCSPPLGR

SEQ ID NO: 640 - protein, 8 aa, synthetic construct
SGGPLGVR

SEQ ID NO: 641 - protein, 8 aa, synthetic construct
SGGPPLGR

SEQ ID NO: 642 - protein, 7 aa, synthetic construct
SGPPLGR

SEQ ID NO: 643 - protein, 4 aa, synthetic construct
SGRS

SEQ ID NO: 644 - protein, 5 aa, synthetic construct
SGRSA

SEQ ID NO: 645 - protein, 6 aa, synthetic construct
SGRSAN

SEQ ID NO: 646 - protein, 7 aa, synthetic construct
SGRSANI

SEQ ID NO: 647 - protein, 7 aa, synthetic construct
SGRSANP

SEQ ID NO: 648 - protein, 9 aa, synthetic construct
SGRSANPRG

SEQ ID NO: 649 - protein, 5 aa, synthetic construct
SGRSD

SEQ ID NO: 650 - protein, 6 aa, synthetic construct
SGRSDD

SEQ ID NO: 651 - protein, 7 aa, synthetic construct
SGRSDDH

SEQ ID NO: 652 - protein, 6 aa, synthetic construct
SGRSDI

SEQ ID NO: 653 - protein, 7 aa, synthetic construct
SGRSDIH

SEQ ID NO: 654 - protein, 6 aa, synthetic construct
SGRSDN

SEQ ID NO: 655 - protein, 7 aa, synthetic construct
SGRSDNI

SEQ ID NO: 656 - protein, 7 aa, synthetic construct
SGRSDNP

SEQ ID NO: 657 - protein, 6 aa, synthetic construct
SGRSDQ

SEQ ID NO: 658 - protein, 7 aa, synthetic construct
SGRSDQH

SEQ ID NO: 659 - protein, 6 aa, synthetic construct
SGRSDT

SEQ ID NO: 660 - protein, 7 aa, synthetic construct
SGRSDTH

SEQ ID NO: 661 - protein, 6 aa, synthetic construct
SGRSDY

SEQ ID NO: 662 - protein, 7 aa, synthetic construct
SGRSDYH

SEQ ID NO: 663 - protein, 5 aa, synthetic construct
SGRSG

SEQ ID NO: 664 - protein, 6 aa, synthetic construct
SGRSGN

SEQ ID NO: 665 - protein, 7 aa, synthetic construct
SGRSGNH

SEQ ID NO: 666 - protein, 8 aa, synthetic construct
SGSPPLGR

SEQ ID NO: 667 - protein, 8 aa, synthetic construct
SIARSDNL

SEQ ID NO: 668 - protein, 14 aa, synthetic construct
SISSGLLSGRSDNI

SEQ ID NO: 669 - protein, 6 aa, synthetic construct
SNPFKY

SEQ ID NO: 670 - protein, 8 aa, synthetic construct
SPLPLRVP

SEQ ID NO: 671 - protein, 8 aa, synthetic construct
SPLPLRVP

SEQ ID NO: 672 - protein, 8 aa, synthetic construct
SPLTGRSG

SEQ ID NO: 673 - protein, 6 aa, synthetic construct
SPPLGR

SEQ ID NO: 674 - protein, 5 aa, synthetic construct
SRRVP

SEQ ID NO: 675 - protein, 6 aa, synthetic construct
SRRVPL

SEQ ID NO: 676 - protein, 6 aa, synthetic construct
SRRVPV

SEQ ID NO: 677 - protein, 8 aa, synthetic construct
SSGPPLGR

SEQ ID NO: 678 - protein, 7 aa, synthetic construct
SSPPLGR

SEQ ID NO: 679 - protein, 8 aa, synthetic construct
SSRGPAYL

SEQ ID NO: 680 - protein, 9 aa, synthetic construct
SSRHRRALD

SEQ ID NO: 681 - protein, 15 aa, synthetic construct
SSSFDKGKYKKGDDA

SEQ ID NO: 682 - protein, 15 aa, synthetic construct
SSSFDKGKYKRGDDA

SEQ ID NO: 683 - protein, 8 aa, synthetic construct
SSSPPLGR

SEQ ID NO: 684 - protein, 8 aa, synthetic construct
STFPFGMF

SEQ ID NO: 685 - protein, 6 aa, synthetic construct
STVFHM

SEQ ID NO: 686 - protein, 6 aa, synthetic construct
SVHHLI

SEQ ID NO: 687 - protein, 8 aa, synthetic construct
SVSGLLSH

SEQ ID NO: 688 - protein, 8 aa, synthetic construct
SVSGLLSS

SEQ ID NO: 689 - protein, 8 aa, synthetic construct
SVSGLRSH

SEQ ID NO: 690 - protein, 8 aa, synthetic construct
SVSGLRSS

SEQ ID NO: 691 - protein, 4 aa, synthetic construct
TARG

SEQ ID NO: 692 - protein, 5 aa, synthetic construct
TARGP

SEQ ID NO: 693 - protein, 12 aa, synthetic construct
TARGPALAHGLF

SEQ ID NO: 694 - protein, 6 aa, synthetic construct
TARGPS

SEQ ID NO: 695 - protein, 7 aa, synthetic construct
TARGPSF

SEQ ID NO: 696 - protein, 8 aa, synthetic construct
TARGPSFK

SEQ ID NO: 697 - protein, 7 aa, synthetic construct
TARGPSW

SEQ ID NO: 698 - protein, 7 aa, synthetic construct
TARGPSW

SEQ ID NO: 699 - protein, 10 aa, synthetic construct
TARGPVPRQV

SEQ ID NO: 700 - protein, 4 aa, synthetic construct
TFVH

SEQ ID NO: 701 - protein, 12 aa, synthetic construct
TGLSGRSVTQTS

SEQ ID NO: 702 - protein, 8 aa, synthetic construct
TGRGPSWV

SEQ ID NO: 703 - protein, 12 aa, synthetic construct
TLRLGRSDNNKN

SEQ ID NO: 704 - protein, 8 aa, synthetic construct
TLSGLRSP

SEQ ID NO: 705 - protein, 8 aa, synthetic construct
TSGRSANP

SEQ ID NO: 706 - protein, 8 aa, synthetic construct
TSGRSGNP

SEQ ID NO: 707 - protein, 12 aa, synthetic construct
TSLSGRSANPRG

SEQ ID NO: 708 - protein, 12 aa, synthetic construct
TSLSGRSGNPRG

SEQ ID NO: 709 - protein, 8 aa, synthetic construct
TSSGLRSP

SEQ ID NO: 710 - protein, 12 aa, synthetic construct
TSTSGRSANPRG

SEQ ID NO: 711 - protein, 22 aa, synthetic construct
TSTSGRSANPRGGGAVGLLAPP

SEQ ID NO: 712 - protein, 24 aa, synthetic construct
TSTSGRSANPRGGGVHMPLGFLGP

SEQ ID NO: 713 - protein, 12 aa, synthetic construct
TSTSGRSGNPRG

SEQ ID NO: 714 - protein, 8 aa, synthetic construct
TVSGLRSP

SEQ ID NO: 715 - protein, 8 aa, synthetic construct
MVLGRSLL

SEQ ID NO: 716 - protein, 8 aa, synthetic construct
SPRSIMLA

SEQ ID NO: 717 - protein, 8 aa, synthetic construct
SMLRSMPL

SEQ ID NO: 718 - protein, 19 aa, synthetic construct
GLSGRSDNHGGAVGLLAPP

SEQ ID NO: 719 - protein, 21 aa, synthetic construct
GLSGRSDNHGGVHMPLGFLGP

SEQ ID NO: 720 - protein, 6 aa, synthetic construct
KLTTVD

SEQ ID NO: 721 - protein, 7 aa, synthetic construct
ASHYFER

SEQ ID NO: 722 - protein, 33 aa, synthetic construct
  VARIANT 6: note = X is D or R
  VARIANT 8..26: note = X is any amino acid or absent
  VARIANT 33: note = X is R or K
KLTTVXXXXXXXXXXXXXXXXXXXXXASHYFEX

SEQ ID NO: 723 - protein, 33 aa, synthetic construct
  VARIANT 8..26: note = X is any amino acid or absent
  VARIANT 33: note = X is R or K
KLTTVDXXXXXXXXXXXXXXXXXXXXASHYFEX

SEQ ID NO: 724 - protein, 33 aa, synthetic construct
  VARIANT 8..26: note = X is any amino acid or absent
  VARIANT 33: note = X is R or K
KLTTVRXXXXXXXXXXXXXXXXXXXXASHYFEX

SEQ ID NO: 725 - protein, 18 aa, synthetic construct
  VARIANT 18: note = X is R or K
KLTTVDGGGGSASHYFEX

SEQ ID NO: 726 - protein, 18 aa, synthetic construct
  VARIANT 18: note = X is R or K
KLTTVRGGGGSASHYFEX

SEQ ID NO: 727 - protein, 38 aa, synthetic construct
  VARIANT 8: note = X is D or R
  VARIANT 10..28: note = X is any amino acid or absent
SQKLTTVXXXXXXXXXXXXXXXXXXXXXASHYFERHLE

SEQ ID NO: 728 - protein, 38 aa, synthetic construct
  VARIANT 10..28: note = X is any amino acid or absent
SQKLTTVDXXXXXXXXXXXXXXXXXXXXASHYFERHLE

SEQ ID NO: 729 - protein, 38 aa, synthetic construct
  VARIANT 10..28: note = X is any amino acid or absent
SQKLTTVRXXXXXXXXXXXXXXXXXXXXASHYFERHLE

SEQ ID NO: 730 - protein, 23 aa, synthetic construct
SQKLTTVDGGGGSASHYFERHLE

SEQ ID NO: 731 - protein, 23 aa, synthetic construct
SQKLTTVRGGGGSASHYFERHLE

SEQ ID NO: 732 - protein, 23 aa, synthetic construct
SQALTTVRGGGGSASHYFERHLE

SEQ ID NO: 733 - protein, 23 aa, synthetic construct
SQALTTVDGGGGSASHYFERHLE

SEQ ID NO: 734 - protein, 18 aa, synthetic construct
KLTTVRGGGGSASHYFER